SEQUENCE LISTING (43 sequences)

SEQ ID NO: 1 - protein, 6 aa, Artificial Sequence
  D-amino acids, lower case letter
MVVYFR

SEQ ID NO: 2 - protein, 7 aa, Artificial Sequence
  D-amino acids, lower case letter
WMVVYFR

SEQ ID NO: 3 - protein, 7 aa, Artificial Sequence
  D-amino acids, lower case letter
KMVVYFR

SEQ ID NO: 4 - protein, 7 aa, Artificial Sequence
  D-amino acids, lower case letter
MVVYFRW

SEQ ID NO: 5 - protein, 7 aa, Artificial Sequence
  D-amino acids, lower case letter
MVVYFRK

SEQ ID NO: 6 - protein, 7 aa, Artificial Sequence
  first amino acid is L-amino acid, other are D-amino acids, lower case letter
WMVVYFR

SEQ ID NO: 7 - protein, 7 aa, Artificial Sequence
  first amino acid is L-amino acid, other are D-amino acids, lower case letter
KMVVYFR

SEQ ID NO: 8 - protein, 7 aa, Artificial Sequence
  last amino acid is L-amino acid, other are D-amino acids, lower case letter
MVVYFRW

SEQ ID NO: 9 - protein, 7 aa, Artificial Sequence
  last amino acid is L-amino acid, other are D-amino acids, lower case letter
MVVYFRK

SEQ ID NO: 10 - protein, 8 aa, Artificial Sequence
  D-amino acids, lower case letter
KWMVVYFR

SEQ ID NO: 11 - protein, 8 aa, Artificial Sequence
  D-amino acids, lower case letter
WKMVVYFR

SEQ ID NO: 12 - protein, 8 aa, Artificial Sequence
  D-amino acids, lower case letter
KKMVVYFR

SEQ ID NO: 13 - protein, 8 aa, Artificial Sequence
  D-amino acids, lower case letter
WWMVVYFR

SEQ ID NO: 14 - protein, 8 aa, Artificial Sequence
  D-amino acids, lower case letter
MVVYFRKW

SEQ ID NO: 15 - protein, 8 aa, Artificial Sequence
  D-amino acids, lower case letter
MVVYFRWK

SEQ ID NO: 16 - protein, 8 aa, Artificial Sequence
  D-amino acids, lower case letter
MVVYFRKK

SEQ ID NO: 17 - protein, 8 aa, Artificial Sequence
  D-amino acids, lower case letter
MVVYFRWW

SEQ ID NO: 18 - protein, 8 aa, Artificial Sequence
  first two amino acids are L-amino acids, other are D-amino acids, lower case letter
KWMVVYFR

SEQ ID NO: 19 - protein, 8 aa, Artificial Sequence
  first two amino acids are L-amino acids, other are D-amino acids, lower case letter
WKMVVYFR

SEQ ID NO: 20 - protein, 8 aa, Artificial Sequence
  first two amino acids are L-amino acids, other are D-amino acids, lower case letter
KKMVVYFR

SEQ ID NO: 21 - protein, 8 aa, Artificial Sequence
  first two amino acids are L-amino acids, other are D-amino acids, lower case letter
WWMVVYFR

SEQ ID NO: 22 - protein, 8 aa, Artificial Sequence
  last two amino acids are L-amino acids, other are D-amino acids, lower case letter
MVVYFRKW

SEQ ID NO: 23 - protein, 8 aa, Artificial Sequence
  last two amino acids are L-amino acids, other are D-amino acids, lower case letter
MVVYFRWK

SEQ ID NO: 24 - protein, 8 aa, Artificial Sequence
  last two amino acids are L-amino acids, other are D-amino acids, lower case letter
MVVYFRKK

SEQ ID NO: 25 - protein, 8 aa, Artificial Sequence
  last two amino acids are L-amino acids, other are D-amino acids, lower case letter
MVVYFRWW

SEQ ID NO: 26 - protein, 9 aa, Artificial Sequence
  D-amino acids, lower case letter
KKWMVVYFR

SEQ ID NO: 27 - protein, 9 aa, Artificial Sequence
  D-amino acids, lower case letter
KWMVVYFRK

SEQ ID NO: 28 - protein, 10 aa, Artificial Sequence
  D-amino acids, lower case letter
KKWMVVYFRK

SEQ ID NO: 29 - protein, 10 aa, Artificial Sequence
  D-amino acids, lower case letter
QKWMVVYFRK

SEQ ID NO: 30 - protein, 15 aa, Artificial Sequence
  D-amino acids, lower case letter
KSQTVKKWMVVYFRK

SEQ ID NO: 31 - protein, 13 aa, Artificial Sequence
  D-amino acids, lower case letter
KKWMVVYFRKSSR

SEQ ID NO: 32 - protein, 13 aa, Artificial Sequence
  D-amino acids, lower case letter
ERSKKWMVVYFRK

SEQ ID NO: 33 - protein, 13 aa, Artificial Sequence
  D-amino acids, lower case letter
KKWMVVYFRKEAR

SEQ ID NO: 34 - protein, 13 aa, Artificial Sequence
  D-amino acids, lower case letter
RSTKKWMVVYFRK

SEQ ID NO: 35 - protein, 12 aa, Artificial Sequence
  D-amino acids, lower case letter
RSTKKWMVVYFR

SEQ ID NO: 36 - protein, 21 aa, Artificial Sequence
  D-amino acids, lower case letter
RASKSQTVKKWMVVYFRKSAR

SEQ ID NO: 37 - protein, 15 aa, Artificial Sequence
  D-amino acids, lower case letter
KSQTVQKWMVVYFRK

SEQ ID NO: 38 - protein, 14 aa, Artificial Sequence
  D-amino acids, lower case letter
SQTVQKWMVVYFRK

SEQ ID NO: 39 - protein, 13 aa, Artificial Sequence
  D-amino acids, lower case letter
SQTVQKWMVVYFR

SEQ ID NO: 40 - protein, 10 aa, Artificial Sequence
  D-amino acids, lower case letter
DKWMVVYFRD

SEQ ID NO: 41 - protein, 13 aa, Artificial Sequence
  D-amino acids, lower case letter
RSTQKWMVVYFRK

SEQ ID NO: 42 - protein, 25 aa, Artificial Sequence
  D-amino acids, lower case letter
KKWMVVYFRKSGSGSKKWMVVYFRK

SEQ ID NO: 43 - protein, 6 aa, Artificial Sequence
  D-amino acids, lower case letter
MVVYFR

(19) United States
(12) Patent Application Publication
(10) Pub. No.: US 2022/0160812 A1
(43) Pub. Date: May 26, 2022

(54) A PHARMACEUTICALLY ACTIVE SUBSTANCE

(71) Applicant: Cytopharma GmbH, Darmstadt (DE)

(72) Inventors: Mark Andre Freyberg, Freiburg (DE); Gerd Klock, Dieburg (DE); Dirk Kaiser, Eppertshausen (DE)

(21) Appl. No.: 17/612,256

(22) PCT Filed: May 18, 2020
(86) PCT No.: PCT/EP2020/063818

§ 371 Date: Nov. 18, 2021

(30) Foreign Application Priority Data
May 22, 2019 (DE) 10 2019 113 612.1

(51) Int. Cl. A61K 38/03; A61K 38/04; A61P 35/00

(52) U.S. Cl. CPC A61K 38/03; A61P 35/00; A61K 38/04

(57) ABSTRACT

The invention relates to a pharmaceutically active substance comprising at least one peptide portion having the amino acid sequences m-v-v-y-f-r (first peptide portion), characterised in that the pharmaceutically active substance comprises at least additional one amino acid and/or peptide portion at the N-terminal end and/or at the C-terminal end of the first peptide portion (second peptide portion and/or third peptide portion) being directly bound to the peptide portion having the amino acid sequences m-v-v-y-f-r (first peptide portion). The invention further relates to a pharmaceutical composition and a drug for the treatment of cancer.

The present invention relates to a pharmaceutically active substance, a drug and a pharmaceutical composition.

Cancer is an abnormal growth of cells which tend to proliferate in an uncontrolled way and, in some cases, to metastasize (spread). Cancer is not one disease. It is a group of more than 100 different and distinctive diseases. Cancer can involve any tissue of the body and have many different forms in each body area. Most cancers are named for the type of cell or organ in which they start. If a cancer spreads (metastasizes), the new tumor bears the same name as the original (primary) tumor.

Typically, cancer can be treated by different options, including chemotherapy, radiation and surgery. Chemotherapy is usually achieved by one or more cytotoxic anti-neoplastic drugs. Traditional cytotoxic agents kill cells that divide rapidly. However, these cytotoxic agents have toxic side effects and their effectiveness is often limited.

The classical chemotherapeutic drugs are cytotoxic and/or prevent cell division which primarily should address tumor cells, presumably while they are proliferating. There are, however, also normal non-tumor cells which proliferate, such as cells of epithelial or mesenchymal origin which reconstitute damaged or aged tissue and anti-cancer drugs could prevent these processes of homeostasis.

A second group of cells which may be stimulated to cell division (proliferation) are immune cells, of the myeloid type or of the lymphocyte type. Immune cells help to fight infections or various origin, microbial, such as bacteria or fungi; or viruses, which may appear due to a new infection or be reactivated, such as viruses of the Herpes virus family. Immune cells may fulfill the task of immune suppression when an infection is under control, or prevent auto-immune reactions or auto-inflammation.

Consequently, these cytotoxic agents have toxic side effects on different cells being very important for the quality of live for human beings having cancer.

In addition thereto, different attempts have been started to treat cancer by providing peptides having an effect on cancer cells. These peptides may introduce apoptosis in cancer cells. E.g. F. Zhong et al. suggest treating lymphocytic leukemia cells by peptide-mediated disruption of Bcl-2-IP₃ receptor interaction (F. Zhong et al., BLOOD, 10 Mar. 2011-VOLUME 117, NUMBER 10; 10.1182/blood-2010-09-307405.). Aline N. Rabaca et al. describe that AC-1001 H3 CDR peptide induces apoptosis and signs of autophagy in vitro and exhibits antimetastatic activity in a syngeneic melanoma model (Aline N. Rabaca et al., FEBS Open Bio 6 (2016) 885-901; doi:10.1002/2211-5463.12080). A. D. Woldetsadik et al. disclose that Hexokinase II-derived cell-penetrating peptide targets mitochondria and triggers apoptosis in cancer cells (A. D. Woldetsadik et al., FASEB J. 31, May (2017); doi: 10.1096/fj.201601173R). Xin Deng et al. suggest peptides with anti-cancer activities. Especially Xin Deng et al. state that B1 and its analogs are capable of penetrating into cytoplasm and triggering cytochrome C release from mitochondria, which ultimately resulted in apoptosis (Xin Deng et al., European Journal of Medicinal Chemistry 89 (2015) 540e548; http://dx.doi.org/10.1016/j.ejmech.2014.10.072). H. Bumpers et al. suggest that Nef-M1, a CXCR4 peptide antagonist, enhances apoptosis and inhibits primary tumor growth and metastasis in breast cancer (H. Bumpers et al., J Cancer Ther. 2013 June; 4(4): 898-906; doi:10.4236/jct.2013.44101). Furthermore, WO 03/054009 provides peptides having apoptotic or anti-apoptotic effects to cells.

The proposals mentioned above have not been applied yet. Nevertheless, targeted therapy is known for the therapy of many cancer types.

For example, a phase I clinical study used cilengitide treatment in a dose escalation study on various brain tumors (NABT 9911). In some of the glioblastoma multiforme (GBM) patients in this study, an indication of response was seen. Cilengitide (=cyclo-(Arg-Gly-Asp-DPhe-NMe-Val), in very marked contrast to most cancer therapeutics currently in use has a very innocuous side effect profile, with no known maximally tolerated dose (MTD) issues in humans—and is very well tolerated.

In addition to the essentially 100% mortality in GBM patients (2-year survival rate about 25%), the morbidity from neurological complications also rapidly degrades the quality of life (QOL).

For example, the standard of treatment of glioblastoma multiforme, associating radiotherapy and temozolomide, has only increased the median survival of resected patients by 2.5 months (from 12.1 to 14.6 months) compared to radiotherapy alone (Stupp et al., 2005). However, in combination with cyclo-(Arg-Gly-Asp-DPhe-NMe-Val), this standard treatment shows significantly improved efficacy with respect to an increased median survival and quality of life.

The annual worldwide incidence of squamous cell cancer of the head and neck (or Squamous Cell Carcinoma of the Head and Neck), both also referred to as SCCHN, is estimated at 500,000 patients; in the United States and Europe, 118.000 new patients are diagnosed annually. SCCHN is more predominant in males with a male:female ratio of 2:1-4:1. There is a positive relationship between smoking habits, alcohol consumption, and head and neck cancer. Approximately 90% of all head and neck malignancies are of squamous cell histology (SCCHN). Most patients are diagnosed with SCCHN at an age of 50-70 years.

A majority of patients (75%) have locally advanced disease at diagnosis. Those patients are mainly treated with radiotherapy and in some cases surgery. Newer strategies such as induction chemotherapy or chemoradiotherapy could provide better survival; however, the 5-year survival rate remains around 30%, and 60% of subjects will experience a loco-regional or distant relapse within 2 years of initial treatment.

The group of subjects with recurrent disease and/or with newly diagnosed distant metastases has very heterogeneous disease characteristics. Their median survival time, however, remains around 6-8 months with a poor quality of life. This dismal prognosis has not changed in the past 30 years.

Lung cancer is the leading cause of cancer deaths worldwide. About 170,000 new cases of lung cancer and 160,000 deaths due to this disease per year occur in the United States alone. Non-small cell carcinoma (NSCLC) accounts for approximately 80% of all lung cancers.

At the time of diagnosis, approximately 30% of NSCLC patients present with locally advanced and 40% with metastatic disease. Surgical results in earlier stages are poor compared to other tumor types (about 40% of recurrence in stages I-II). In metastatic disease, chemotherapy is the treatment of choice, but survival benefits have been modest, resulting in one-year survival of 40%, and five-year survival of less than 15%.

It is commonly accepted that the standard treatment for advanced disease (stage IV and IIIb with malignant pleural effusion) consists of platin-based (cisplatin or carboplatin) chemotherapy. However, there are many open questions in the management of these patients, such as the role of combination therapy regimen including more than two drugs, non-platinum-based therapies, and new targeted therapeutical approaches.

Currently, response rates of about 20%-30% and median survival times of 6 to 11 months have been observed in the treatment of metastatic NSCLC. Several chemotherapy combinations are used with comparable efficacy. Accordingly, also in this field is a high unmet medical need for improved methods of treatment.

Small cell lung cancer (SCLC) accounts for 15-20% of all lung cancer cases in the world, equating to approximately 80,000 new patients every year. A recent analysis of the Surveillance, Epidemiology and End Results database confirmed that in the United States, the proportion of small cell lung cancer patients has decreased from about 20% to 13.8% in 1998, likely due to the implementation of smoking cessation programs. This success, however, is to some extent outweighed by the high and rising prevalence of tobacco smoking in other parts of the world.

SCLC is typically disseminated at the time of presentation, with approximately 60% to 70% of patients having disseminated (extensive-stage) disease at presentation. Thus, surgery is rarely an option, and applies only to patients with localized (limited) disease. Relapse and death from SCLC is imminent even in patients who are treated with surgical resection. Without other therapy than surgery, survival was 2 months for patients with extensive-stage SCLC and 3 months for patients with limited-stage SCLC (Green, Am J Med 1969).

Systemic combination chemotherapy remains the mainstay of SCLC treatment, both in limited and extensive stage of their disease. For more than 20 years, etoposide and cis-/carboplatin are considered the current standard agents used in combination for the first-line treatment of patients with SCLC in the Western world. Combination therapy with more than two drugs in clinical trials has resulted in higher response rates, but also higher toxicity, and did not result in a clinically relevant overall survival benefit. Time to progression is short, with the majority of patients progressing within 3 months of completing chemotherapy. The median survival is 7 to 11 months. Less than 5% of patients survive longer than 2 years.

The term breast cancer or malignant breast neoplasm is commonly used as the generic name for cancers originating from breast tissue, most commonly from the inner lining of milk ducts or the lobules that supply the ducts with milk. Cancers originating from ducts are often referred to as ductal carcinomas; those originating from lobules are often referred to as lobular carcinomas. However, there are many different types of breast cancer, with different stages (spread), aggressiveness, and genetic makeup; survival varies greatly depending on those factors. Breast cancer is about 100 times more common in women than in men, although males tend to have poorer outcomes due to delays in diagnosis.

Breast cancer (BRCA) is the most common cancer in women worldwide, accounting for 18 30% of all female cancers. It represents a major public health problem mainly due to its high incidence, excess mortality and therapeutic challenges. More than 1.1 million women are diagnosed with BRCA each year worldwide, and more than 400.000 succumb to this disease. Approximately 75% of all newly diagnosed patients are women with early stage BRCA.

Generally, treatment options include surgery, drug based therapy, including but not limited to hormonal therapy and/or chemotherapy, and radiation. Some breast cancers require hormones to grow, such as estrogen and/or progesterone, and have receptors for those hormones. After surgery those cancers are treated with drugs that interfere with those hormones and/or shut off the production of said hormones in the ovaries or elsewhere. Such drugs are generally referred to as hormone antagonists or hormone blockers.

However, despite surgery and the use of adjuvant treatments such as chemotherapy, hormonal therapy, radiotherapy and targeted drugs, many of these patients will die as a result of local or distant recurrence. The 5-year survival rate for metastatic breast cancer is in the range of 25%.

As can be seen from the above, management of cancer has been difficult and it still is difficult.

Thus, even in view of the results achieved within the last years, the prognosis of the patients regarding most cancerous diseases is still very grim. There still exists a growing need in the art in order to develop new pharmaceuticals, including new compounds and advantageous formulations of non-compounds for treating cancer and/or metastases thereof. Preferably, said new pharmaceuticals should allow for convenient and/or efficacious systemic application or administration. Thus, there is a need for improved drugs, medicaments, therapy methods and treatment regimen.

An object of the present invention therefore was to provide a new compound, preferably a new and advantageous drug and/or a new and advantageous pharmaceutical composition. It should preferably be applicable to systemic treatment, preferably lower the dose and/or preferably increase the efficiency of the pharmaceutical to be applied and/or allow for a more convenient administration and/or dosing regimen.

Thus, there is a high medical need to provide a more effective, better tolerated method for the treatment of subjects, preferably mammalian subjects, more preferably human subjects humans and especially human cancer patients that may be suffering from various cancers and/or metastasis thereof, thus preferably also leading to enhanced progression-free survival (PFS), improved quality of life (QOL) and/or increased median survival.

These objects and further objects which are not stated explicitly but which are immediately derivable or discernible from the connections discussed herein by way of introduction are solved by a pharmaceutically active substance having all features of claim 1.

The present invention accordingly provides a pharmaceutically active substance comprising at least one peptide portion having the amino acid sequences m-v-v-y-f-r (first peptide portion), characterized in that the pharmaceutically active substance comprises at least additional one amino acid and/or peptide portion at the N-terminal end and/or at the C-terminal end of the first peptide portion (second peptide portion and/or third peptide portion) being directly bound to the peptide portion having the amino acid sequences m-v-v-y-f-r (first peptide portion). The expression “being directly bound” is well known in the art and specifies that at the N-terminal end and/or C-terminal end a bond is formed connecting the N-terminal end and/or C-terminal end with an amino acid and/or peptide portion.

It is thus possible in an unforeseeable manner to improve the methods for the treatment of cancer as mentioned above. The present invention accordingly provides a pharmaceutically active substance, preferably an apoptotically active substance, preferably a drug, more preferably a pharmaceutical composition leading to enhanced progression-free survival (PFS), improved quality of life (QOL) and/or increased median survival. Surprisingly, the pharmaceutically active substance, preferably the apoptotically active substance, preferably the drug, more preferably the pharmaceutical composition is well-tolerated and has low side effects.

Moreover, certain immune cells such as T cells (T lymphocytes, CD4⁺ or CD8⁺), and natural killer (NK) cells may be activated to directly attack tumor cells or to support the fight against the tumor. In addition, other cell types such as dendritic cells, monocytes, macrophages and NKT cells may help in the process of tumor attack. The present invention provides a treatment wherein the therapy facilitates the natural recognition and attack of tumor cells by the immune system. Therefore, the immune system is not suppressed by the present pharmaceutically active substances, preferably the apoptotically active substances, preferably drugs, more preferably pharmaceutical compositions but have synergistic effect using the immune systems of persons in need.

Furthermore, as shown in the Examples the pharmaceutically active substances, preferably the apoptotically active substances, preferably drugs, more preferably pharmaceutical compositions of the present invention have a high effectivity with regard to very different cancer cell types. That is, the pharmaceutically active substances, preferably apoptotically active substances, preferably drugs, more preferably pharmaceutical compositions can be successfully applied in order to treat very different cancers. Based on the pathway of action, the cancer cells having a high resistance against conventional chemotherapeutics can be effectively treated using the pharmaceutically active substances, preferably apoptotically active substances, preferably drugs, more preferably pharmaceutical compositions of the present invention.

On the other hand, as shown in the Examples the pharmaceutically active substances, preferably apoptotically active substances, preferably drugs, more preferably pharmaceutical compositions show a high selectivity and, hence, have low side effects against healthy cells.

The present invention provides therapies which show partial or complete specificity for (direct or indirect) eradication of cancer cells, without attacking normal cells including immune cells, and irrespective of whether the normal cells are in a proliferating or resting (non-dividing) state.

The present invention provides pharmaceutically active substances, preferably apoptotically active substances exhibiting a very high efficiency and a very rapid effect, preferably as pharmaceutical against cancer. Although the pharmaceutically active substances comprises peptides which may have a relatively low half-value time and may be depleted at relatively high speed, the very rapid effect of the pharmaceutically active substances overcompensate these issues. The very rapid effect of the pharmaceutically active substances, preferably apoptotically active substances is very astonishing and is never achieved by any other drug for treatment of cancer. This surprising efficiency regarding the astonishing fastness of the killing of cancer cells improves the survival rates of persons in need.

A combination of such desirable features are not achieved using conventional methods as described above.

The pharmaceutically active substances, preferably apoptotically active substances can be obtained very inexpensively.

The present invention provides pharmaceutically active substances. Pharmaceutically active substances are considered as substances being useful for the production of pharmaceuticals and include especially active ingredients. Without being limited thereto, it is believed that pharmaceutically active substances are able to induce apoptosis. Apoptosis is well known in the art. Apoptosis is the genetically encoded suicide program, which is induced in eukaryotic cells under certain physiological or pathological conditions. For the purposes of the present invention, “apoptotically active” is understood to mean that the addition of the corresponding substance to the test system shown in present Examples generates a positive or negative effect to the survival of the cells. Preferably, the present substances induce apoptosis and provide a high level of apoptosis (cell death based on suicide program) with regard to cells being prone to apoptosis such as cancer cells while healthy cells are not or only at a low level affected by the substances of the present invention. The apoptosis can be measured by conventional methods being well known in the art such as microscopy and the methods mentioned in the Examples. Although microscopic methods provide most reliable results, for the purpose of the present invention more convenient methods can be applied.

For achieving a high reliability and preciseness, the apoptosis can be measured by microscopy. For achieving reliable results a high number of cells should be used to determine the apoptosis rate as mentioned above and below. Preferably, at least 1000 cell, more preferably at least 10000 cells and even more preferably at least 1000000 cells are used to determine the apoptosis rate. The cells being analysed by conventional microscopy and preferably automatic picture analyses can be performed. The cells, preferably the cell nucleus being stained by a conventional dye. Cells, especially cell nucleus having a healthy shape (round or spherical shape) and cells, especially cell nucleus having a apoptotic shape (necking, narrowing or any other shape indicating apoptosis as known in the art) are counted. Cells having a necrotic or other non-apoptotic or non-healthy cell shape are considered. Death cells having a non-apoptotic shape, such as necrotic shape can be determined by a staining with propidium iodide. Conventionally, healthy cells and apoptotic cells have a cell wall in good order while necrotic cells have a permeable cell wall. Consequently, for purpose of the present invention, cells having a permeable cell wall are not considered as healthy cells or apoptotic cells.

Preferably the pharmaceutically active substances, preferably apoptotically active substances inducing apoptosis provide an inducing level of apoptosis of at least 5%, more preferably of at least 10%, even more preferably of at least 30% and even more preferably of at least 40%. The level of apoptosis of apoptosis is preferably measured by microscopy and is determined by the number of the measured apoptotic cells on the basis of the measured total cells.

Preferably the pharmaceutically active substances, preferably apoptotically active substances inducing apoptosis provide an inducing level of apoptosis of at least 5%, more preferably of at least 10%, even more preferably of at least 30% and even more preferably of at least 40%. The level of apoptosis of apoptosis is preferably measured by microscopy and is determined by the number of the measured apoptotic cells on the basis of the measured healthy cells and apoptotic cells.

The level of apoptosis of at least 5%, more preferably of at least 10%, even more preferably of at least 30% and even more preferably of at least 40% is preferably achieved at a concentration of the pharmaceutically active substance according to the invention of at most 500 μM, preferably of at most 200 μM and even more preferably of at most 100 μM. Preferably level of apoptosis of at least 5%, more preferably of at least 10%, even more preferably of at least 30% and even more preferably of at least 40% is preferably achieved using cells of the cell lines A375, BxPC-3, MEC-1, MCF-7, MDA-MB-231, CACO-2, MKN-45, CAL-29, HeLa, RPMI-8226, BHT-101, BFTC-909, A-431, CAL-27, DU-145, EFE-184, FU-OV-1, HCC-33, HEP-3B, KYSE-140, ML-1, TFK-1, SK-GT-2, RPMI-2650 and/or A-549 more preferably under the condition as shown in the Examples.

As shown in the Examples, the level of apoptosis may depend on the incubation time. According to a preferred embodiment, the level of apoptosis as mentioned above and below refers to the maximum value achieved by an incubation time in the range of 1.5 to 24 hours, more preferably under the condition as shown in the Examples. According to a very preferred embodiment, the level of apoptosis as mentioned above and below refers to an incubation time of about 3 hours.

The cells of the cell lines A375, BxPC-3, MEC-1, MCF-7, MDA-MB-231, CACO-2, MKN-45, CAL-29, HeLa, RPMI-8226, BHT-101, BFTC-909, A-431, CAL-27, DU-145, EFE-184, FU-OV-1, HCC-33, HEP-3B, KYSE-140, ML-1, TFK-1, SK-GT-2, RPMI-2650 and/or A-549 are well known in the art and are commercially available from DSMZ Braunschweig (Leibniz Institut DSMZ-Deutsche Sammlung von Mikroorganismen and Zellkulturen GmbH), Germany.

The level of apoptosis of at least 5%, more preferably of at least 10%, even more preferably of at least 30% and even more preferably of at least 40% is preferably achieved using the cell line A375 at a concentration of the pharmaceutically active substance according to the invention of 500 μM or below preferably measured by microscopy and is determined by the number of the measured apoptotic cells on the basis of the measured total cells.

The level of apoptosis of at least 4%, more preferably of at least 8%, even more preferably of at least 30% and even more preferably of at least 40% is preferably achieved using the cell line A375 at a concentration of the pharmaceutically active substance according to the invention of 200 μM or below preferably measured by microscopy and is determined by the number of the measured apoptotic cells on the basis of the measured total cells.

The level of apoptosis of at least 3%, more preferably of at least 6%, even more preferably of at least 15% and even more preferably of at least 30% is preferably achieved using the cell line A375 at a concentration of the pharmaceutically active substance according to the invention of 100 μM or below measured by microscopy and is determined by the number of the measured apoptotic cells on the basis of the measured total cells.

The level of apoptosis of at least 5%, more preferably of at least 10%, even more preferably of at least 30% and even more preferably of at least 40% is preferably achieved using the cell line BxPC-3 at a concentration of the pharmaceutically active substance according to the invention of 500 μM or below preferably measured by microscopy and is determined by the number of the measured apoptotic cells on the basis of the measured total cells.

The level of apoptosis of at least 4%, more preferably of at least 8%, even more preferably of at least 30% and even more preferably of at least 40% is preferably achieved using the cell line BxPC-3 at a concentration of the pharmaceutically active substance according to the invention of 200 μM or below preferably measured by microscopy and is determined by the number of the measured apoptotic cells on the basis of the measured total cells.

The level of apoptosis of at least 3%, more preferably of at least 6%, even more preferably of at least 15% and even more preferably of at least 30% is preferably achieved using the cell line BxPC-3 at a concentration of the pharmaceutically active substance according to the invention of 100 μM or below preferably measured by microscopy and is determined by the number of the measured apoptotic cells on the basis of the measured total cells.

The level of apoptosis of at least 5%, more preferably of at least 10%, even more preferably of at least 30% and even more preferably of at least 40% is preferably achieved using the cell line MEC-1 at a concentration of the pharmaceutically active substance according to the invention of 500 μM or below preferably measured by microscopy and is determined by the number of the measured apoptotic cells on the basis of the measured total cells.

The level of apoptosis of at least 4%, more preferably of at least 8%, even more preferably of at least 30% and even more preferably of at least 40% is preferably achieved using the cell line MEC-1 at a concentration of the pharmaceutically active substance according to the invention of 200 μM or below preferably measured by microscopy and is determined by the number of the measured apoptotic cells on the basis of the measured total cells.

The level of apoptosis of at least 3%, more preferably of at least 6%, even more preferably of at least 15% and even more preferably of at least 30% is preferably achieved using the cell line MEC-1 at a concentration of the pharmaceutically active substance according to the invention of 100 μM or below preferably measured by microscopy and is determined by the number of the measured apoptotic cells on the basis of the measured total cells.

Preferably, the proportion of cells which has died are preferably determined by two methods, both rely on the analysis of cells by flow cytometry, often called FACS analysis. This technology allows determining the physical and chemical properties of both normal cells and tumor cells. The cells can be labelled by fluorescent dyes, which may bind to cellular DNA or which are coupled to antibodies that bind to proteins or other structures on the cell surface or inside the cells. The cells are suspended and injected into a flow cytometer instrument and, ideally one at a time, the cells flow through a laser beam. The laser light is either scattered or elicits fluorescence at the dyes which were applied and the scattered and the fluorescent light is analyzed by an optical and light detection system. This system with an array of dichroic mirrors or prisms and optical filters serves to direct the light to photomultipliers that are mounted in defined positions, either in line of the laser and the position where the cell meets the laser beam, thereby delivering signals of the forward-scattered light (FSC); or photomultipliers detect light which is scattered in an angle relative to the FSC beam direction, to detect the side-scattered light (SSC), or to detect selected wave length from fluorescent light. Programmed cell death (apoptosis) can be detected by changes of the FSC/SSC light scattering pattern, or by the appearance of cells with a low DNA content, as revealed by the so called subG1 peak (Darzynkiewicz et al., (1992). “Features of apoptotic cells measured by flow cytometry.” Cytometry 13(8): 795-808; Vermes et al., (2000) “Flow cytometry of apoptotic cell death.” J Immunol Methods 243(1-2): 167-190).

A first method is based on the cell form measurement, herein called Vital Cells analysis or cell vitality. The basis of this method has been published, e.g. by the documents of Lizard, G. et al. (1995). “Kinetics of plasma membrane and mitochondrial alterations in cells undergoing apoptosis.” Cytometry 21(3): 275-283, doi: 10.1002/cyto.990210308; Taga et al. (2000) “Contribution of automated hematology analysis to the detection of apoptosis in peripheral blood lymphocytes.” Cytometry 42(3): 209-214; Vermes et al., (2000) “Flow cytometry of apoptotic cell death.” J Immunol Methods 243(1-2): 167-190.

As disclosed in more detail in the Examples, the analyzation includes a selected cell fraction excluding cells with both small FSC and small SSC values. Therefore, a gating area is drawn in a two-dimensional FSC/SSC plot (region R-1) which contains mainly intact cells, but no small vesicles. This is initially done for each cell type using reference cells which were not incubated with substances inducing cell death. The geometry applied for region R-1 is preferably a pentagon, as shown in FIG. 1.

Preferably the coordinates for the corner points (m1 to m5) are in the following ranges, given as percent of full scale for SSC (S) and FSC (F), respectively:

First point of pentagon m1 preferably ranges for SSC (S) from 2.5% to 25.0%, more preferably from 5.0% to 20.0%, more preferably about 15%.

First point of pentagon m1 preferably ranges for FSC (F) from 12.0% to 55.0, more preferably from 15.0% to 25.0%, more preferably about 20%. F=%).

Second point of pentagon m2 preferably ranges for SSC (S) from 25.0% to 70.0%, more preferably from 32.0% to 45.0%, more preferably about 37%.

Second point of pentagon m2 preferably ranges for FSC (F) from 25.0% to 45.0, more preferably from 30.0% to 44.0%, more preferably about 36%.

Third point of pentagon m3 preferably ranges for SSC (S) from 30.0% to 70.0%, more preferably from 55.0% to 67.0%, more preferably about 66%.

Third point of pentagon m3 preferably ranges for FSC (F) from 50.0% to 65.0, more preferably from 52.0% to 60.0%, more preferably about 56%.

Fourth point of pentagon m4 preferably ranges for SSC (S) from 10.0% to 60.0%, more preferably from 13.0% to 30.0%, more preferably about 21%.

Fourth point of pentagon m4 preferably ranges for FSC (F) from 42.0% to 80.0, more preferably from 55.0% to 75.0%, more preferably about 65%.

Fifth point of pentagon m5 preferably ranges for SSC (S) from 2.0% to 40.0%, more preferably from 6.0% to 20.0%, more preferably about 13%.

Fifth point of pentagon m5 preferably ranges for FSC (F) from 25.0% to 45.0, more preferably from 30.0% to 40.0%, more preferably about 35%.

As mentioned above and below, the data for assessing the geometry applied for region R-1 are rough estimations and are preferably optimized as mentioned in the Examples and the documents cited above and below in order to achieve a region R-1 containing mainly intact cells, but no small vesicles. This is initially done for each cell type using reference cells which were not incubated with substances inducing cell death. By this selection of the cell fraction, a bias is prevented which would include a very high number of small cellular vesicles (apoptotic bodies) that are formed by membrane blebbing from dying cells, which have a low DNA content but which, by their large number, would increase the fraction of presumptive subG1 cells.

A second method is based on the analysis of the cell cycle phases which can be drawn from the relative DNA content. Following flow cytometry, the frequency of fluorescence intensities which is proportional to the DNA content is plotted in a single dimension histogram, which reveals whether the cells are either in the G₀/G1 phase (resting cells); or the cells proliferate and are in the DNA synthesis (S) phase or in the G₂/M phase which is before or at the time of cell division (mitosis). When cells die by apoptosis, cellular changes lead to diminished DNA content resulting in a new peak located before the G₀/G1 peak, often called “subG1” peak (Broecker-Preuss et al. (2015) “Cell death induction by the BH3 mimetic GX15-070 in thyroid carcinoma cells.” J Exp Clin Cancer Res 34: 69; Darzynkiewicz et al., (1992) “Features of apoptotic cells measured by flow cytometry.” Cytometry 13(8): 795-808; Dbaibo et al. (1998) “p53-dependent ceramide response to genotoxic stress.” J Clin Invest 102(2): 329-339; Fischbeck et al. (2011). “Sphingomyelin induces cathepsin D-mediated apoptosis in intestinal epithelial cells and increases inflammation in DSS colitis.” Gut 60(1): 55-65). The second method is herein called SubG1 method.

According to a preferred embodiment, the apoptosis level is measured according to the cell cycle SubG1 method being explained in more detail in the Examples and the documents mentioned above published by Darzynkiewicz, Z. et al. (1992). “Features of apoptotic cells measured by flow cytometry.” Cytometry 13(8): 795-808, doi: 10.1002/cyto.990130802, where cellular DNA was stained with DAPI; and Broecker-Preuss, M. et al. (2015). “Cell death induction by the BH3 mimetic GX15-070 in thyroid carcinoma cells.” J Exp Clin Cancer Res 34: 69, doi: 10.1186/s13046-015-0186-x, for staining with propidium iodide (PI); both methods allow to quantify the cell cycle subG1 fraction with increased DNA content typical for apoptotic cells.

According to another preferred embodiment, the apoptosis level is based on the levels of vital cells and non-vital cells (including apoptotic cells) measured according to the FACS method being explained in more detail in the Examples and the documents mentioned above published by Lizard, G. et al. (1995). “Kinetics of plasma membrane and mitochondrial alterations in cells undergoing apoptosis.” Cytometry 21(3): 275-283, doi: 10.1002/cyto.990210308; and Vermes, I. et al. (2000). “Flow cytometry of apoptotic cell death.” J Immunol Methods 243(1-2): 167-190. (Vital Cells analysis or cell vitality)

Preferably the pharmaceutically active substances, preferably apoptotically active substances inducing apoptosis provide an inducing level of apoptosis of at least 5%, more preferably of at least 10%, even more preferably of at least 30% and even more preferably of at least 40%. The level of apoptosis of apoptosis is preferably measured according to the SubG1 method and is determined by the number of the measured dead cells on the basis of the measured total cells.

Preferably the pharmaceutically active substances, preferably apoptotically active substances inducing apoptosis provide an inducing level of apoptosis of at least 5%, more preferably of at least 10%, even more preferably of at least 30% and even more preferably of at least 40%. The level of apoptosis of apoptosis is preferably measured according to the cell vitality method and is determined by the number of the measured dead cells on the basis of the measured total cells.

The level of apoptosis of at least 5%, more preferably of at least 10%, even more preferably of at least 30% and even more preferably of at least 40% is preferably achieved at a concentration of the pharmaceutically active substance according to the invention of at most 500 μM, preferably of at most 200 μM and even more preferably of at most 100 μM. Preferably level of apoptosis of at least 5%, more preferably of at least 10%, even more preferably of at least 30% and even more preferably of at least 40% is preferably achieved using cells of the cell lines A375, BxPC-3, MEC-1, MCF-7, MDA-MB-231, CACO-2, MKN-45, CAL-29, HeLa, RPMI-8226, BHT-101, BFTC-909, A-431, CAL-27, DU-145, EFE-184, FU-OV-1, HCC-33, HEP-3B, KYSE-140, ML-1, TFK-1, SK-GT-2, RPMI-2650 and/or A-549 more preferably under the condition as shown in the Examples.

As shown in the Examples, the level of apoptosis may depend on the incubation time. According to a preferred embodiment, the level of apoptosis as mentioned above and below refers to the maximum value achieved by an incubation time in the range of 1.5 to 24 hours, more preferably under the condition as shown in the Examples. According to a very preferred embodiment, the level of apoptosis as mentioned above and below refers to an incubation time of about 3 hours.

The cells of the cell lines A375, BxPC-3, MEC-1, MCF-7, MDA-MB-231, CACO-2, MKN-45, CAL-29, HeLa, RPMI-8226, BHT-101, BFTC-909, A-431, CAL-27, DU-145, EFE-184, FU-OV-1, HCC-33, HEP-3B, KYSE-140, ML-1, TFK-1, SK-GT-2, RPMI-2650 and/or A-549 are well known in the art and are commercially available from DSMZ Braunschweig (Leibniz Institut DSMZ-Deutsche Sammlung von Mikroorganismen and Zellkulturen GmbH), Germany.

The level of apoptosis of at least 5%, more preferably of at least 10%, even more preferably of at least 30% and even more preferably of at least 40% is preferably achieved using the cell line A375 at a concentration of the pharmaceutically active substance according to the invention of 500 μM or below preferably according to the SubG1 method and/or cell vitality method, more preferably according to the SubG1 method.

The level of apoptosis of at least 4%, more preferably of at least 8%, even more preferably of at least 30% and even more preferably of at least 40% is preferably achieved using the cell line A375 at a concentration of the pharmaceutically active substance according to the invention of 200 μM or below preferably according to the SubG1 method and/or cell vitality method, more preferably according to the SubG1 method.

The level of apoptosis of at least 3%, more preferably of at least 6%, even more preferably of at least 15% and even more preferably of at least 30% is preferably achieved using the cell line A375 at a concentration of the pharmaceutically active substance according to the invention of 100 μM or below preferably according to the SubG1 method and/or cell vitality method, more preferably according to the SubG1 method.

The level of apoptosis of at least 5%, more preferably of at least 10%, even more preferably of at least 30% and even more preferably of at least 40% is preferably achieved using the cell line BxPC-3 at a concentration of the pharmaceutically active substance according to the invention of 500 μM or below preferably according to the SubG1 method and/or cell vitality method, more preferably according to the SubG1 method.

The level of apoptosis of at least 4%, more preferably of at least 8%, even more preferably of at least 30% and even more preferably of at least 40% is preferably achieved using the cell line BxPC-3 at a concentration of the pharmaceutically active substance according to the invention of 200 OA or below preferably according to the SubG1 method and/or cell vitality method, more preferably according to the SubG1 method.

The level of apoptosis of at least 3%, more preferably of at least 6%, even more preferably of at least 15% and even more preferably of at least 30% is preferably achieved using the cell line BxPC-3 at a concentration of the pharmaceutically active substance according to the invention of 100 μM or below preferably according to the SubG1 method and/or cell vitality method, more preferably according to the SubG1 method.

The level of apoptosis of at least 5%, more preferably of at least 10%, even more preferably of at least 30% and even more preferably of at least 40% is preferably achieved using the cell line MEC-1 at a concentration of the pharmaceutically active substance according to the invention of 500 OA or below preferably according to the SubG1 method and/or cell vitality method, more preferably according to the SubG1 method.

The level of apoptosis of at least 4%, more preferably of at least 8%, even more preferably of at least 30% and even more preferably of at least 40% is preferably achieved using the cell line MEC-1 at a concentration of the pharmaceutically active substance according to the invention of 200 OA or below preferably according to the SubG1 method and/or cell vitality method, more preferably according to the SubG1 method.

The level of apoptosis of at least 3%, more preferably of at least 6%, even more preferably of at least 15% and even more preferably of at least 30% is preferably achieved using the cell line MEC-1 at a concentration of the pharmaceutically active substance according to the invention of 100 OA or below preferably according to the SubG1 method and/or cell vitality method, more preferably according to the SubG1 method.

Preferably, the second peptide portion and/or third peptide portion comprises at least one alpha-amino acid within a sequence of 5 amino acid units from the first peptide portion, preferably within a sequence of 3 amino acid units from the first peptide portion the additional amino acid and/or peptide portion.

In preferred embodiments the second peptide portion and/or third peptide portion comprises at least one polar amino acid within a sequence of 5 amino acid units from the first peptide portion, preferably within a sequence of 3 amino acid units from the first peptide portion. Preferably, the second peptide portion and/or third peptide portion comprising at least two, preferably at least 3 polar amino acid within a sequence of 5 amino acid units from the first peptide portion, preferably within a sequence of 3 amino acid units from the first peptide portion.

Preferably, it can be provided that the second peptide portion and/or third peptide portion comprises at least one basic amino acid within a sequence of 5 amino acid units from the first peptide portion, preferably within a sequence of 3 amino acid units from the first peptide portion. Preferably, the second peptide portion and/or third peptide portion comprises at least two, preferably at least 3 basic amino acid within a sequence of 5 amino acid units from the first peptide portion, more preferably within a sequence of 3 amino acid units from the first peptide portion.

In a further preferred embodiment it can be provided that the second peptide portion and/or third peptide portion comprises more polar amino acids than nonpolar amino acids within a sequence of 5 amino acid units from the first peptide portion.

Preferably, the second peptide portion and/or third peptide portion comprises the same number or more of basic amino acids than acidic amino acids within a sequence of 5 amino acid units from the first peptide portion.

The expressions nonpolar amino acid, polar amino acid, acidic amino acid and basic amino acid are well known in the art. Nonpolar amino acid are amino acid having no polar group in the side chain such as alanine, cysteine, glycine, isoleucine, leucine, methionine, phenylalanine, proline, tryptophan and/or valine.

Polar amino acid are amino acid having at least one polar group in the side chain such as arginine, asparagine, aspartic add, glutamine, glutamic acid, histidine, lysine, serine, threonine and/or tyrosine.

Acidic amino acid are amino acid having at least one acidic group in the side chain, such as aspartic acid and/or glutamic acid.

Basic amino acid are amino acid having at least one basic group in the side chain, such as arginine, histidine and/or lysine.

Acidic amino acids and basic amino acids are polar amino acids.

In preferred embodiments the apoptotically active substance comprises two additional amino acid and/or peptide portions, one at the N-terminal end and one at the C-terminal end of the peptide portion having the amino acid sequences m-v-v-y-f-r (second peptide portion and third portion) being directly bound to the peptide portion having the amino acid sequences m-v-v-y-f-r (first peptide portion).

The second peptide portion is preferably located at the N-terminal end of the first peptide portion and the third peptide portion is preferably located at the C-terminal end of the first peptide portion. The second peptide portion preferably comprises at least one amino acid residue, more preferably at least two amino residues. The third peptide portion preferably comprises at least one amino acid residue.

For the purposes of the present invention, the internationally customary single letter codes for amino acids will be used, which is to say A denotes alanine (Ala), C cysteine (Cys), D aspartic acid (Asp), E glutamic acid (Glu), F phenylalanine (Phe), G glycine (Gly), L leucine (Leu), M methionine (Met), N asparagine (Asn), P proline (Pro), R arginine (Arg), S serine (Ser), T threonine (Thr), V valine (Val), W tryptophan (Trp), and Y tyrosine ((Tyr). L-amino acids are symbolized here by capital letters, and D-amino acids are symbolized by the use of lower case letters.

The amino acids are preferably selected from naturally occurring amino acids, synthetic amino acids and/or synthetically modified naturally occurring amino acids. Naturally occurring amino acids, synthetic amino acids and/or synthetically modified naturally occurring amino acids are known to the skilled artisan. Preferably, said naturally occurring amino acids, synthetic amino acids and/or synthetically modified naturally occurring amino acids are as defined herein.

Generally, the term “non-naturally occurring amino acids” is preferably intended to include any small molecule having at least one carboxyl group and at least one primary or secondary amino group capable of forming a peptide bond. The term “peptide” is preferably intended to include any molecule having at least one peptide bond. The term “peptide” preferably also embraces structures as defined above having one or more linkers, spacers, terminal groups or side chain groups which are not amino acids.

According to the invention, the naturally occurring amino acids are preferably selected from the group consisting of Gly, Ala, β-Ala, Asn, Asp, Arg, Cys, Gln, Glu, His, Ile, Leu, Lys, Met, Nle, Orn, Phe, Pro, Ser, Thr, Trp, Tyr and Val and more preferably selected from the L forms thereof.

According to the invention, the non-naturally occurring amino acids or synthetically modified naturally occurring amino acids are preferably selected from the group consisting of:

i) the D forms of naturally occurring amino acids, i.e. the D forms of Gly, Ala, β-Ala, Asn, Asp, Arg, Cys, Gin, Glu, His, Ile, Leu, Lys, Met, Nle, Orn, Phe, Pro, Ser, Thr, Trp, Tyr and Val,

ii) the N-alkyl derivatives of Gly, Ala, β-Ala, Asn, Asp, Arg, Cys, Gln, Glu, His, Ile, Leu, Lys, Met, Nle, Orn, Phe, Pro, Ser, Thr, Trp, Tyr and Val, preferably including both the D and L forms thereof, and

iii) Lys(Ac), Lys(AcNH₂), Lys(AcSH), Tic, Asp(OR), Cha, NaI, 4-HaI-Phe, homo-Phe, Phg, Pya, Abu, Acha, Acpa, Aha, Ahds, Aib, Aos, N-Ac-Arg, Dab, Dap, Deg, hPro, Nhdg, homoPhe, 4-HaI-Phe, Phg, Sar, Tia, Tic and Ile, preferably including both the D and L forms thereof;

wherein

R is alkyl having 1-18 carbon atoms, preferably alkyl having 1-6 carbon atoms and especially alkyl having 1-4 carbon atoms,

HaI is F, Cl, Br, I

Ac is alkanoyl having 1-10 and more preferably 1-6 carbon atoms, aroyl having 7-11 carbon atoms or aralkanoyl having 8-12 carbon atoms.

With respect to the N-alkyl derivatives of said amino acids, alkyl is preferably selected from methyl, ethyl, isopropyl, n-butyl, sec-butyl and tert-butyl. However, alkyl is furthermore also preferably selected from n-pentyl, isopentyl, neopentyl, n-hexyl, n-heptyl, n-octyl, n nonyl, n-decyl and n-hexadecyl.

According to the invention, the non-naturally occurring amino acids are preferably selected from the group consisting of the D forms of naturally occurring amino acids, i.e. the D forms of Gly, Ala, β-Ala, Asn, Asp, Arg, Cys, Gin, Glu, His, Ile, Leu, Lys, Met, Nle, Orn, Phe, Pro, Ser, Thr, Trp, Tyr and Val.

According to the invention, the synthetically modified naturally occurring amino acids are preferably selected from the group consisting of the N-alkyl derivatives of the L forms of Gly, Ala, 1a-Ala, Asn, Asp, Arg, Cys, Gln, Glu, His, Ile, Leu, Lys, Met, Nle, Orn, Phe, Pro, Ser, Thr, Trp, Tyr and Val, wherein the N-alkyl residues preferably consist of 1-18 carbon atoms, more preferably 1-6 carbon atoms and even more preferably 1-4 carbon atoms.

According to the invention, the synthetically modified naturally occurring amino acids are preferably selected from the group consisting of the N-methyl derivatives and/or N-ethyl derivatives of the L forms of Gly, Ala, β-Ala, Asn, Asp, Arg, Cys, Gin, Glu, His, Ile, Leu, Lys, Met, Nle, Orn, Phe, Pro, Ser, Thr, Trp, Tyr and Val. Especially preferably, the synthetically modified naturally occurring amino acids are selected from the group consisting of the L forms of N-Methyl-Gly, N-Methyl-Ala, N-Methyl-β-Ala, N-Methyl-Asn, N-Methyl-Asp, N-Methyl-Arg, N-Methyl-Cys, N-Methyl-Gln, N-Methyl-Glu, N-Methyl-His, N-Methyl-Ile, N-Methyl-Leu, N-Methyl-Lys, N-Methyl-Met, N-Methyl-Nle, N-Methyl-Orn, N-Methyl-Phe, N-Methyl-Pro, N-Methyl-Ser, N-Methyl-Thr, N-Methyl-Trp, N-Methyl-Tyr and N-Methyl-Val, which are preferably also referred to as NMeGly, NMeAla, NMeβ-Ala, NMeAsn, NMeAsp, NMeArg, NMeCys, NMeGln, NMeGlu, NMeHis, NMelle, NMeLeu, NMeLys, NMeMet, NMeNle, NMeOrn, NMePhe, NMePro, NMeSer, NMeThr, NMeTrp, NMeTyr and NMeVai.

According to the invention, the synthetically modified naturally occurring amino acids are preferably selected from the group consisting of the N-alkyl derivatives of the D forms of Gly, Ala, β-Ala, Asn, Asp, Arg, Cys, Gin, Glu, His, lie, Leu, Lys, Met, Nle, Orn, Phe, Pro, Ser, Thr, Trp, Tyr and Val, wherein the N-alkyl residues preferably consist of 1-18 carbon atoms, more preferably 1-6 carbon atoms and even more preferably 1-4 carbon atoms.

According to the invention, the synthetically modified naturally occurring amino acids are preferably selected from the group consisting of the N-methyl derivatives and/or N-ethyl derivatives of the D forms of Gly, Ala, β-Ala, Asn, Asp, Arg, Cys, Gin, Glu, His, Ile, Leu, Lys, Met, Nle, Orn, Phe, Pro, Ser, Thr, Trp, Tyr and Val. Especially preferably, the synthetically modified naturally occurring amino acids are selected from the group consisting of the D forms of N-Methyl-Gly, N-Methyl-Ala, N-Methyl-β-Ala, N-Methyl-Asn, N-Methyl-Asp, N-Methyl-Arg, N-Methyl-Cys, N-Methyl-Gln, N-Methyl-Glu, N-Methyl-His, N-Methyl-lie, N-Methyl-Leu, N-Methyl-Lys, N-Methyl-Met, N-Methyl-Nle, N-Methyl-Orn, N-Methyl-Phe, N-Methyl-Pro, N-Methyl-Ser, N-Methyl-Thr, N-Methyl-Trp, N-Methyl-Tyr and N-Methyl-Val, which are preferably also referred to as NMeGly, NMeAla, NMeβ-Ala, NMeAsn, NMeAsp, NMeArg, NMeCys, NMeGln, NMeGlu, NMeHis, NMelle, NMeLeu, NMeLys, NMeMet, NMeNle, NMeOrn, NMePhe, NMePro, NMeSer, NMeThr, NMeTrp, NMeTyr and NMeVal.

Preferably, it can be provided that the second peptide portion and/or third peptide portion comprises at least one D-amino acid. Preferably, at least 50%, preferably at least 70% of the second peptide portion and/or third peptide portion are D-amino acids.

In preferred embodiments the apoptotically active substance comprises at least one peptide portion having the amino acid sequences (SEQ ID No 1) to (SEQ ID No 8)

(SEQ ID No 1)
w-m-v-v-y-f-r,
(SEQ ID No 2)
k-m-v-v-y-f-r,
(SEQ ID No 3)
m-v-v-y-f-r-w,,
(SEQ ID No 4)
m-v-v-y-f-r-k,
(SEQ ID No 5)
W-m-v-v-y-f-r,
(SEQ ID No 6)
K-m-v-v-y-f-r,
(SEQ ID No 7)
m-v-v-y-f-r-W,
(SEQ ID No 8)
m-v-v-y-f-r-K.

Regarding the sequences mentioned above, the sequences consisting of D-amino acids such as (SEQ ID No 1) to (SEQ ID No 4) (w-m-v-v-y-f-r, k-m-v-v-y-f-r, m-v-v-y-f-r-w, m-v-v-y-f-r-k) are preferred. The sequences having the (SEQ ID No 1) and/or (SEQ ID No 2) (w-m-v-v-y-f-r and k-m-v-v-y-f-r) are especially preferred. The sequence having the (SEQ ID No 1) (w-m-v-v-y-f-r) is most preferred with regard to the sequences mentioned above.

In a further preferred embodiment it can be provided that the apoptotically active substance comprises at least one peptide portion having the amino acid sequences

(SEQ ID No 9)
k-w-m-v-v-y-f-r,
(SEQ ID No 10)
w-k-m-v-v-y-f-r,
(SEQ ID No 11)
k-k-m-v-v-y-f-r,
(SEQ ID No 12)
w-w-m-v-v-y-f-r,
(SEQ ID No 13)
m-v-v-y-f-r-k-w,
(SEQ ID No 14)
m-v-v-y-f-r-w-k,
(SEQ ID No 15)
m-v-v-y-f-r-k-k,
(SEQ ID No 16)
m-v-v-y-f-r-w-w,
(SEQ ID No 17)
K-W-m-v-v-y-f-r,
(SEQ ID No 18)
W-K-m-v-v-y-f-r,
(SEQ ID No 19)
K-K-m-v-v-y-f-r,
(SEQ ID No 20)
W-W-m-v-v-y-f-r,
(SEQ ID No 21)
m-v-v-y-f-r-K-W,
(SEQ ID No 22)
m-v-v-y-f-r-W-K,
(SEQ ID No 23)
m-v-v-y-f-r-K-K,
(SEQ ID No 24)
m-v-v-y-f-r-W-W.

Regarding the sequences mentioned above, the sequences consisting of D-amino acids such as (SEQ ID No 9) to (SEQ ID No 16) (k-w-m-v-v-y-f-r, w-k-m-v-v-y-f-r, k-k-m-v-v-y-f-r, w-w-m-v-v-y-f-r, m-v-v-y-f-r-k-w, m-v-v-y-f-r-w-k, m-v-v-y-f-r-k-k, m-v-v-y-f-r-w-w) are preferred. The sequences having the (SEQ ID No 9) to (SEQ ID No 12) (w-m-v-v-y-f-r, w-k-m-v-v-y-f-r, k-k-m-v-v-y-f-r and w-w-m-v-v-y-f-r) are especially preferred, even more preferably the sequences having the (SEQ ID No 9) and (SEQ ID No 10) (k-w-m-v-v-y-f-r and w-k-m-v-v-y-f-r). The sequence having the (SEQ ID No 9) (k-w-m-v-v-y-f-r) is most preferred with regard to the sequences mentioned above.

Preferably, the apoptotically active substance comprises at least one peptide portion having the amino acid sequence

(SEQ ID No 25)
k-k-w-m-v-v-y-f-r,
(SEQ ID No 26)
k-w-m-v-v-y-f-r-k,

or a sequence having a homology to said sequences

(SEQ ID No 25)
k-k-w-m-v-v-y-f-r,
(SEQ ID No 26)
k-w-m-v-v-y-f-r-k,

of at least 30%, preferably at least 60%, based on the underlined amino acids.

More preferably, the apoptotically active substance comprises at least one peptide portion having the amino acid sequence

(SEQ ID No 27)
k-k-w-m-v-v-y-f-r-k,
(SEQ ID No 28)
q-k-w-m-v-v-y-f-r-k,

or a sequence having a homology to said sequences

(SEQ ID No 27)
k-k-w-m-v-v-y-f-r-k,
(SEQ ID No 28)
q-k-w-m-v-v-y-f-r-k,

of at least 30%, preferably at least 50%, more preferably at least 70%, based on the underlined amino acids.

Surprisingly, the sequence having the (SEQ ID No 27), (SEQ ID No 28) or a sequence having a homology to said sequences of at least 50%, preferably at least 70%, based on the underlined amino acids as mentioned above and below (k-k-w-m-v-v-y-f-r-k and g-k-w-m-v-v-y-f-r-k) show improvements regarding a higher specificity and higher efficiency in view of the peptides mentioned above such as sequence having the (SEQ ID No 25), (SEQ ID No 26) or a sequence having a homology to said sequences of at least 50%, preferably at least 70%, based on the underlined amino acids as mentioned above and below (k-k-w-m-v-v-y-f-r and k-w-m-v-v-y-f-r-k). That is healthy cells are effected at a lower level using the sequences having the (SEQ ID No 27), (SEQ ID No 28) or a sequence having a homology to said sequences of at least 50%, preferably at least 70%, based on the underlined amino acids as mentioned above and below (k-k-w-m-v-v-y-f-r-k and g-k-w-m-v-v-y-f-r-k) in comparison to the sequences having the (SEQ ID No 25), (SEQ ID No 26) or a sequence having a homology to said sequences of at least 50%, preferably at least 70%, based on the underlined amino acids as mentioned above and below (k-k-w-m-v-v-y-f-r and k-w-m-v-v-y-f-r-k). Furthermore, the sequences having the (SEQ ID No 27), (SEQ ID No 28) or a sequence having a homology to said sequences of at least 50%, preferably at least 70%, based on the underlined amino acids as mentioned above and below (k-k-w-m-v-v-y-f-r-k and g-k-w-m-v-v-y-f-r-k) have a higher ability to induce apoptosis in cancer cells than the sequences having the (SEQ ID No 25), (SEQ ID No 26) or a sequence having a homology to said sequences of at least 50%, preferably at least 70%, based on the underlined amino acids as mentioned above and below (k-k-w-m-v-v-y-f-r and k-w-m-v-v-y-f-r-k).

The expression “homology” is well known in the art. The term “percent homology” means the percentage of identical amino acid residues based on the underlined amino acids. That is, a homology of at least 50% means that at most 2 of the underlined 4 amino acids of a sequence, e. g. the sequence k-k-w-m-v-v-y-f-r-k (e. g. SEQ ID No 27) could be substituted by any other amino acid. Preferably the substituted amino acid residue is an amino acid residue having a high similarity. Amino acids having a high similarity are selected from the same group as the amino acid residue being substituted. That is, if the tryptophan is substituted, the amino acid residue substituting the tryptophan is preferably an aromatic amino acid residue such as phenylalanine or tyrosine. Regarding the groups for determining the similarity, in the present invention the groups for determining the similarity are based on the side chain classes, including the amino acid classes “acid”, “aliphatic”, “amide”, “aromatic”, “basic”, “cyclic”, “hydroxyl-containing” and/or “sulfur-containing”. More preferably, a specific D-amino acid is preferably substituted by another D-amino acid. That is, e.g. in the sequence k-k-w-m-v-v-y-f-r-k (e. g. SEQ ID No 27) the underlined lysine is preferably substituted by an arginine residue being in the D form and the tryptophan is preferably substituted by His, Phe, Tyr, if a derivate of the sequence k-k-w-m-v-v-y-f-r-k (e. g. SEQ ID No 27) is applied.

The amino add class “acid” include the amino acids Asp and Glu. The amino acid class “aliphatic” include the amino acids Ala, Gly, Ile, Leu and Val. The amino acid class “amide” include the amino acids Asn and Gln. The amino add class “aromatic” include the amino acids His, Phe, Trp and Tyr. The amino acid class “basic” include the amino acids Arg, His and Lys. The amino add class “cyclic” includes the amino acid Pro. The amino acid class “hydroxyl-containing” include the amino acids Ser and Thr. The amino acid class “sulfur-containing” include the amino acids Cys and Met.

As mentioned above, the sequence having the (SEQ ID No 27), (SEQ ID No 28) or a sequence having a homology to said sequences show improvements regarding a high specificity and high efficiency.

The efficiency preferably concerns the level of apoptosis as mentioned above and below. The selectivity concerns the ratio of the apoptosis of cancer cells to the apoptosis of healthy cells. Preferably the ratio is at least 1:1, preferably at least 2:1 and more preferably at least 3:1.

According to a specific embodiment, the selectivity is based on the ratio of the apoptosis of cancer cells to the apoptosis of human peripheral blood mononuclear cells (“PBMC”), more preferably Lymphocyte cells. Preferably the inventive pharmaceutically active substance provides a selectivity of at least 1:1, preferably at least 2:1 and more preferably at least 3:1, being based the ratio of the apoptosis of cancer cells to the apoptosis of Lymphocyte cells. More preferably the selectivity is based on a concentration of about 50 μM or above, even more preferably of at least 100 μM and most preferably of 200 μM. The selectivity and the concentration are disclosed in more details in the Examples. Preferably, the apoptosis is measured by microscopy. Preferably the apoptosis is measured according to the cell cycle analysis (subG1 values) and/or percentage of cells in the region R-1 in the diagram (application Forward Scatter versus Sideward Scatter) (Determination of cell vitality), more preferably by determination of cell vitality.

According to a preferred embodiment, the selectivity is based on the ratio of the apoptosis of MEC-1 cells to the apoptosis of human peripheral blood mononuclear cells (“PBMC”), more preferably Lymphocyte cells. Preferably the inventive pharmaceutically active substance provides a selectivity of at least 1:1, preferably at least 2:1 and more preferably at least 3:1, being based the ratio of the apoptosis of MEC-1 cells to the apoptosis of Lymphocyte cells. More preferably the selectivity is based on a concentration of about 50 μM or above, even more preferably of at least 100 μM and most preferably of 200 μM. The selectivity and the concentration are disclosed in more details in the Examples. Preferably, the apoptosis is measured by microscopy. Preferably the apoptosis is measured according to the cell cycle analysis (subG1 values) and/or percentage of cells in the region R-1 in the diagram (application Forward Scatter versus Sideward Scatter) (Determination of cell vitality), more preferably by determination of cell vitality.

According to a preferred embodiment, the selectivity is based on the ratio of the apoptosis of BxPC-3 cells to the apoptosis of human peripheral blood mononuclear cells (“PBMC”), more preferably Lymphocyte cells. Preferably the inventive pharmaceutically active substance provides a selectivity of at least 1:1, preferably at least 2:1 and more preferably at least 3:1, being based the ratio of the apoptosis of BxPC-3 cells to the apoptosis of Lymphocyte cells. More preferably the selectivity is based on a concentration of about 50 μM or above, even more preferably of at least 100 μM and most preferably of 200 μM. The selectivity and the concentration are disclosed in more details in the Examples. Preferably the apoptosis is measured according to the cell cycle analysis (subG1 values) and/or percentage of cells in the region R-1 in the diagram (application Forward Scatter versus Sideward Scatter) (Determination of cell vitality), more preferably by determination of cell vitality.

According to a preferred embodiment, the selectivity is based on the ratio of the apoptosis of A375 cells to the apoptosis of human peripheral blood mononuclear cells (-PBMC″), more preferably Lymphocyte cells. Preferably the inventive pharmaceutically active substance provides a selectivity of at least 1:1, preferably at least 2:1 and more preferably at least 3:1, being based the ratio of the apoptosis of A375 cells to the apoptosis of Lymphocyte cells. More preferably the selectivity is based on a concentration of about 50 μM or above, even more preferably of at least 100 μM and most preferably of 200 μM. The selectivity and the concentration are disclosed in more details in the Examples. Preferably the apoptosis is measured according to the cell cycle analysis (subG1 values) and/or percentage of cells in the region R-1 in the diagram (application Forward Scatter versus Sideward Scatter) (Determination of cell vitality), more preferably by determination of cell vitality.

In a further preferred embodiment it can be provided that the apoptotically active substance comprises at least one peptide portion having at least one of the amino acid sequences

(SEQ ID No 29)
k-s-q-t-v-k-k-w-m-v-v-y-f-r-k,
(SEQ ID No 30)
k-k-w-m-v-v-y-f-r-k-s-s-r,
(SEQ ID No 31)
e-r-s-k-k-w-m-v-v-y-f-r-k,
(SEQ ID No 32)
k-k-w-m-v-v-y-f-r-k-e-a-r,
(SEQ ID No 33)
r-s-t-k-k-w-m-v-v-y-f-r-k,
(SEQ ID No 34)
r-s-t-k-k-w-m-v-v-y-f-r,
(SEQ ID No 35)
r-a-s-k-s-q-t-v-k-k-w-m-v-v-y-f-r-k-s-a-r,

or a sequence having a homology to said sequences

(SEQ ID No 29)
k-s-q-t-v-k-k-w-m-v-v-y-f-r-k,
(SEQ ID No 30)
k-k-w-m-v-v-y-f-r-k-s-s-r,
(SEQ ID No 31)
e-r-s-k-k-w-m-v-v-y-f-r-k,
(SEQ ID No 32)
k-k-w-m-v-v-y-f-r-k-e-a-r,
(SEQ ID No 33)
r-s-t-k-k-w-m-v-v-y-f-r-k,
(SEQ ID No 34)
r-s-t-k-k-w-m-v-v-y-f-r,,
(SEQ ID No 35)
r-a-s-k-s-q-t-v-k-k-w-m-v-v-y-f-r-k-s-a-r

of at least 50%, preferably at least 70%, based on the underlined amino acids.

The sequences having the (SEQ ID No 29), (SEQ ID No 30), (SEQ ID No 32), (SEQ ID No 33), (SEQ ID No 35) (k-s-q-t-v-k-k-w-m-v-v-y-f-r-k, k-k-w-m-v-v-y-f-r-k-s-s-r, r-s-t-k-k-w-m-v-v-y-f-r-k and k-k-w-m-v-v-y-f-r-k-e-a-r, r-a-s-k-s-q-t-v-k-k-w-m-v-v-y-f-r-k-s-a-r) are especially preferred, more preferably the sequences having the (SEQ ID No 29), (SEQ ID No 30), (SEQ ID No 33), (SEQ ID No 35) (r-a-s-k-s-q-t-v-k-k-w-m-v-v-y-f-r-k-s-a-r, k-k-w-m-v-v-y-f-r-k-s-s-r, r-s-t-k-k-w-m-v-v-y-f-r-k and k-s-q-t-v-k-k-w-m-v-v-y-f-r-k), even more preferably the sequence having the (SEQ ID No 33) r-s-t-k-k-w-m-v-v-y-f-r-k) or (SEQ ID No 29) (k-s-q-t-v-k-k-w-m-v-v-y-f-r-k). Furthermore, sequences having a high homology to said sequences are also preferred. That is, a sequence having a homology of at least 50%, preferably at least 70%, based on the underlined amino acids to the sequences having the (SEQ ID No 29), (SEQ ID No 30), (SEQ ID No 32), (SEQ ID No 33), (SEQ ID No 35)

(k-s-q-t-v-k-k-w-m-v-v-y-f-r-k,
k-k-w-m-v-v-y-f-r-k-s-s-r,
r-s-t-k-k-w-m-v-v-y-f-r-k,
k-k-w-m-v-v-y-f-r-k-e-a-r,
r-a-s-k-s-q-t-v-k-k-w-m-v-v-y-f-r-k-s-a-r)

are especially preferred, more preferably the sequences having the (SEQ ID No 29), (SEQ ID No 30), (SEQ ID No 33), (SEQ ID No 35)

(r-a-s-k-s-q-t-v-k-k-w-m-v-v-y-f-r-k-s-a-r,
k-k-w-m-v-v-y-f-r-k-s-s-r,
r-s-t-k-k-w-m-v-v-y-f-r-k and
k-s-q-t-v-k-k-w-m-v-v-y-f-r-k),

even more preferably the sequence having the (SEQ ID No 33) (r-s-t-k-k-w-m-v-v-y-f-r-k) or (SEQ ID No 29) k-s-t-v-k-k-w-m-v-v-y-f-r-k.

In a further preferred embodiment it can be provided that the apoptotically active substance comprises at least one peptide portion having at least one of the amino acid sequences

(SEQ ID No 36)
k-s-q-t-v-q-k-w-m-v-v-y-f-r-k,
(SEQ ID No 37)
s-q-t-v-q-k-w-m-v-v-y-f-r-k,
(SEQ ID No 38)
s-q-t-v-q-k-w-m-v-v-y-f-r,

or a sequence having a homology to said sequences

(SEQ ID No 36)
k-s-q-t-v-q-k-w-m-v-v-y-f-r-k,
(SEQ ID No 37)
s-q-t-v-q-k-w-m-v-v-y-f-r-k,
(SEQ ID No 38)
s-q-t-v-q-k-w-m-v-v-y-f-r,

of at least 50%, preferably at least 70%, based on the underlined amino acids.

The sequences having the (SEQ ID No 37) and (SEQ ID No 36) (k-s-q-t-v-q-k-w-m-v-v-y-f-r-k and s-q-t-v-q-k-w-m-v-v-y-f-r-k) are especially preferred, even more preferably the sequence having the (SEQ ID No 36) (k-s-q-t-v-q-k-w-m-v-v-y-f-r-k). Furthermore, sequences having a high homology to said sequences are also preferred. That is, a sequence having a homology of at least 50%, preferably at least 70%, based on the underlined amino acids to the sequences having the (SEQ ID No 37) and (SEQ ID No 36)

(k-s-q-t-v-q-k-w-m-v-v-y-f-r-k and
s-q-t-v-q-k-w-m-v-v-y-f-r-k)

are preferred, more preferably to the sequence having the (SEQ ID No 36)

k-s-q-t-v-q-k-w-
m-v-v-y-f-r-k.

Although the sequence having the (SEQ ID No 27) k-k-w-m-v-v-y-f-r-k and their derivatives having a homology of at least 50%, preferably at least 70%, based on the underlined amino acids (k-k-w-m-v-v-y-f-r-k) as mentioned above have a high efficiency, the sequences having the (SEQ ID No 38), (SEQ ID No 37) and (SEQ ID No 36) (s-q-t-v-q-k-w-m-v-v-y-f-r, s-q-t-v-q-k-w-m-v-v-y-f-r-k and k-s-q-t-v-q-k-w-m-v-v-y-f-r-k) are preferred. Furthermore sequences having a high homology to the sequences having the (SEQ ID No 38), (SEQ ID No 37) and (SEQ ID No 36)

(s-q-t-v-q-k-w-m-v-v-y-f-r,
s-q-t-v-q-k-w-m-v-v-y-f-r-k and
k-s-q-t-v-q-k-w-m-v-v-y-f-r-k)

are preferred over sequences having a high homology to the sequence having the (SEQ ID No 27) (k-k-w-m-v-v-y-f-r-k), based on the underlined amino acids.

In a further embodiment of the present invention the apoptotically active substance comprises at least one peptide portion having the amino acid sequence d-k-w-m-v-v-y-f-r-d (SEQ ID No 39), or a sequence having a homology to the sequence d-k-w-m-v-v-y-f-r-d (SEQ ID No 39) at least 30%, preferably at least 50%, more preferably at least 70%, based on the underlined amino acids. These sequences have a high efficiency. However, the sequences having the (SEQ ID No 27), (SEQ ID No 28) and (SEQ ID No 38) (s-q-t-v-q-k-w-m-v-v-y-f-r, k-k-w-m-v-v-y-f-r-k and q-k-w-m-v-v-y-f-r-k) are preferred over the sequence having the (SEQ ID No 39) (d-k-w-m-v-v-y-f-r-d). Furthermore sequences having a high homology to the sequences having the (SEQ ID No 27), (SEQ ID No 28) and (SEQ ID No 38) (s-q-t-v-q-k-w-m-v-v-y-f-r, k-k-w-m-v-v-y-f-r-k and g-k-w-m-v-v-y-f-r-k) are preferred over sequences having a high homology to the sequence having the (SEQ ID No 39) (d-k-w-m-v-v-y-f-r-d), based on the underlined amino acids.

In a further embodiment of the present invention the apoptotically active substance comprises at least one peptide portion having the amino acid sequence r-s-t-q-k-w-m-v-v-y-f-r-k (SEQ ID No 40), or a sequence having a homology to the sequence r-s-t-q-k-w-m-v-v-y-f-r-k (SEQ ID No 40) at least 30%, preferably at least 50%, more preferably at least 70%, based on the underlined amino acids. These sequences have a high efficiency. The sequence having the SEQ ID No 40 (r-s-t-q-k-w-m-v-v-y-f-r-k) has about the same efficiency as the sequence having the (SEQ ID No 33) (r-s-t-k-k-w-m-v-v-y-f-r-k). Furthermore sequences having a high homology to the sequences having the (SEQ ID No 40) (r-s-t-q-k-w-m-v-v-y-f-r-k) have about the same efficiency as the sequences having a high homology to the sequence having the (SEQ ID No 33) (r-s-t-k-k-w-m-v-v-y-f-r-k), based on the underlined amino acids.

In view of efficiency as agent for inducing apoptosis, the sequences having the (SEQ ID No 29), (SEQ ID No 37) and (SEQ ID No 36) (k-s-q-t-v-q-k-w-m-v-v-y-f-r-k, s-q-t-v-q-k-w-m-v-v-y-f-r-k and k-s-q-t-v-k-k-w-m-v-v-y-f-r-k) are preferred in view of other sequences mentioned above and the sequence having the (SEQ ID No 36) (k-s-q-t-v-q-k-w-m-v-v-y-f-r-k) is most preferred. The same applies to their homologues. That is sequences having homology of at least 50%, preferably at least 70%, based on the underlined amino acids to the sequences having the (SEQ ID No 29), (SEQ ID No 37) and (SEQ ID No 36)

(k-s-q-t-v-q-k-w-m-v-v-y-f-r-k,
s-q-t-v-q-k-w-m-v-v-y-f-r-k and
k-s-q-t-v-k-k-w-m-v-v-y-f-r-k)

are preferred over other sequences as mentioned above and sequences having homology of at least 50%, preferably at least 70%, based on the underlined amino acids to the sequence (SEQ ID No 36) are preferred over other sequences.

The apoptotically active substance of the present invention can comprise a single peptide as mentioned above and below. That is the apoptotically active substance comprises exactly one first peptide portion and exactly one second peptide portion, the apoptotically active substance comprises exactly one first peptide portion and exactly one third peptide portion or the apoptotically active substance comprises exactly one first peptide portion, exactly one second peptide portion and exactly one third peptide portion.

In a preferred embodiment the apoptotically active substance of the present invention preferably comprises at least one of the sequences having the (SEQ ID No 9) to SEQ ID No 39), preferably (SEQ ID No 27) to SEQ ID No 39), more preferably (SEQ ID No 29) to SEQ ID No 38), even more preferably (SEQ ID No 29) to SEQ ID No 37), even more preferably (SEQ ID No 29), (SEQ ID No 37) and (SEQ ID No 36).

In a preferred embodiment, the apoptotically active substance of the present invention preferably comprises two, three, four, five or more peptides (or peptide portions) as mentioned above. More preferably, the pharmaceutically active substance comprises two, three, four, five or more of the first peptide portion, of the second peptide portion and/or of the third peptide portion. Even more preferably, pharmaceutically active substance comprises two, three, four, five or more of the first peptide portion. It should be noted that the second peptide portion and/or third peptide portion) being directly bound to the peptide portion having the amino acid sequences m-v-v-y-f-r (first peptide portion) can be the same as the peptide portion having the amino acid sequences m-v-v-y-f-r. Astonishingly, the pharmaceutically active substance having two, three, four, five or more of the first peptide portion having the amino acid sequences m-v-v-y-f-r shows an astonishing and unexpected improvement over the peptide having the amino acid sequences m-v-v-y-f-r with no additional amino acids.

That is, in a preferred embodiment the apoptotically active substance of the present invention preferably comprises at least two of the peptide sequences mentioned above and below, more preferably peptide sequences having the sequence ID No 1 to No 40, even more preferably peptide sequences having the sequence ID No 1 to No 39 mentioned above and below. The at least two peptide sequences comprised by the apoptotically active substance of the present invention can be the same or different in that embodiment.

In a further embodiment of the present invention the apoptotically active substance of the present invention preferably comprises at least two of the sequences having the (SEQ ID No 9) to SEQ ID No 40), preferably (SEQ ID No 9) to SEQ ID No 39), even more preferably (SEQ ID No 27) to SEQ ID No 40), even more preferably (SEQ ID No 27) to SEQ ID No 39), even more preferably (SEQ ID No 29) to (SEQ ID No 38) and (SEQ ID No 40), even more preferably (SEQ ID No 29) to SEQ ID No 38), even more preferably (SEQ ID No 29) to (SEQ ID No 37) and (SEQ ID No 40), even more preferably (SEQ ID No 29) to (SEQ ID No 37), even more preferably (SEQ ID No 29), (SEQ ID No 30), (SEQ ID No 33) and (SEQ ID No 35) (SEQ ID No 37) and (SEQ ID No 36) and (SEQ ID No 40), even more preferably (SEQ ID No 29), (SEQ ID No 30), (SEQ ID No 33) and (SEQ ID No 35) (SEQ ID No 37) and (SEQ ID No 36), even more preferably (SEQ ID No 29), (SEQ ID No 37) and (SEQ ID No 36), even more preferably the at least two sequences being present in the apoptotically active substance of the present invention are identical. That is, in a very preferred embodiment, the apoptotically active substance of the present invention comprises at least two sequences having the (SEQ ID No 36).

Even more preferably the apoptotically active substance of the present invention preferably comprises exactly two, three, four, five, six or seven of the peptide sequences mentioned above and below, more preferably peptide sequences having the sequence ID No 1 to No 40, even more preferably peptide sequences having the sequence ID No 1 to No 39 mentioned above and below. In one embodiment of the present invention, wherein the apoptotically active substance of the present invention preferably comprises at least two of the pepfide sequences mentioned above and below, more preferably peptide sequences having the sequence ID No 1 to No 40 mentioned above and below, even more preferably peptide sequences having the sequence ID No 1 to No 39 mentioned above and below, the apoptotically active substance comprises at least two identical peptide sequences mentioned above and below, more preferably peptide sequences having the sequence ID No 1 to No 40 mentioned above and below, even more preferably peptide sequences having the sequence ID No 1 to No 39 mentioned above and below. In another embodiment of the present invention, wherein the apoptotically active substance of the present invention preferably comprises at least two of the peptide sequences mentioned above and below, more preferably peptide sequences having the sequence ID No 1 to No 40 mentioned above and below, even more preferably peptide sequences having the sequence ID No 1 to No 39 mentioned above and below, the apoptotically active substance comprises at least two different peptide sequences mentioned above and below, more preferably peptide sequences having the sequence ID No 1 to No 40 mentioned above and below, even more preferably peptide sequences having the sequence ID No 1 to No 39 mentioned above and below.

According to a preferred embodiment the apoptotically active substance preferably comprises a peptide chain being formed by the first peptide portion and the second and/or third peptide portion and the peptide chain has a chain length of <100 amino acids, more preferably the apoptotically active substance comprises a peptide chain being formed by the first peptide portion and the second and/or third peptide portion and the peptide chain has a chain length of <50 amino acids. For the purposes of the present invention, the term “peptide” is understood to mean a substance that comprises a chain of 2 or more amino acids linked by peptide bonds.

Peptides for use in the pharmaceutically active substance according to the invention preferably comprise 7 to 100 amino acids, more preferably 8 to 50 and especially 9 to 40 amino acids.

Preferably, the apoptotically active substance is a peptide that has a chain length of <100, preferably <50 amino acids.

For the purposes of the present invention, the term “protein” is understood to mean a substance in which multiple “peptides” are linked to each other, for example by molecular bonds such as disulphide bonds or by salt bridges. This definition equally encompasses native proteins as well as at least partially “artificial” proteins, wherein such “artificial” proteins may be modified, for example by attaching chemical groups to the amino acid chain that normally do not occur in native proteins.

Native peptides, however, often have a low metabolic stability with respect to peptidases and relatively poor bioavailability.

Proceeding from the above-described peptides, a person skilled in the art, without exercising inventive skill, will be able to develop a whole number of derived compounds that have a similar or identical mode of action and, among other things, are also referred to as peptide mimetics.

For the purposes of the present invention, peptide mimetics refer to compounds that mimic the structure of peptides and, serving as ligands, are able to imitate (agonist) or block (antagonist) the biological activity at the receptor/enzyme level. Above all, the peptide mimetics are to have improved bioavailability and improved metabolic stability. The manner of mimetization can range from a slightly modified original structure to a pure non-peptide, see, for example, A. Adang et al., Recl. Tray. Chim. Pays-Bas 113 (1994), 63-78.

In principle, the following mimetization/derivatization options of a peptide structure are available:

use of D- instead of L-amino acids

modification of the side chain of amino acids

alteration/extension of the peptide main chain

cyclization for conformational stabilization

use of templates that forcibly create a certain secondary structure use of a non-peptide backbone, which together with suitable groups/side chains mimics the structure of the peptide

While it is possible to enhance the proteolytic stability of a peptide by exchanging L- with D-amino acids, or by exchanging D- with L-amino acids, modifying the side chains of one of the amino acids often improves the binding properties of the entire peptide.

An alteration of the peptide backbone generally involves exchanging an amide group with amide-like groups (J. Gante, Angew. Chem. 106 (1994), 1780-1802). This measure allows both the binding affinity and the metabolic stability of the native peptide to be influenced.

Cyclization of a linear peptide establishes the flexibility of the same, and hence the global conformation. When the biologically active conformation is fixed, the affinity of the peptide for the receptor is increased since the decrease in entropy in the bond is less than in the case of the bond of a flexible, linear peptide. For this purpose, amino acid side chains not involved in receptor identification are linked to each other or to the peptide scaffold.

The secondary structure of the peptide plays a crucial role for the molecular identification of the receptor. In addition to a-helix and β-sheet, what are known as turns form important conformation elements in the peptide chain. Replacing these structural units with a building block that, after insertion into a peptide, stabilizes a defined secondary structure, resulted in the concept of secondary structure mimetics.

The water solubility of the peptides can also be increased, for example by introducing S- and C-glycopeptide derivatives. Further measures can be the PEGylation of the peptides, for example.

It can be provided that the apoptotically active substance comprises a peptide chain being formed by the first peptide portion and the second and/or third peptide portion and the peptide chain preferably has a solubility in water of at least 2 WI, preferably at least 4 g/l.

It can be provided that the peptide chain being formed by the first peptide portion and the second and/or third peptide portion and the peptide chain preferably has a solubility in water of at least 2 g/I, preferably at least 4 g/l.

For some applications and/or formulation the apoptotically active substance comprises a peptide chain being formed by the first peptide portion and the second and/or third peptide portion comprises a portion providing improved hydrophobicity. Such portion providing improved hydrophobicity is especially useful for forming a complex or other adduct with some proteins being able to carry the apoptotically active substance of the present invention to the cancer cells being prone to apoptosis and to improve the life time of the apoptotically active substance within the body of subjects, preferably mammalian subjects, more preferably human subjects humans and especially human cancer patients.

Preferably, the portion providing improved hydrophobicity is a fatty acid residue and/or an alkyl residue having about 5 to 40 C-atoms, more preferably about 10 to 30 C-atoms, even more preferably about 12 to 24 C-atoms. The fatty acid residue and/or an alkyl residue can be saturated or unsaturated and linear or branched. Preferred fatty acid residues are e.g. hexanoate, heptanoate, octanoate, nonanoate, decanoate, undecanoate, dodecanoate, tridecanoate, tetradecanoate, pentadecanoate, hexadecanoate, heptadecanoate, octadecanoate, nonadecanoate, icosanoate, myristate, laurate, palmitate, stearate, and/or oleate. Preferred alkyl residues are e.g. hexyl, heptyl, octyl, nonyl, decyl, undecyl, dodecyl (lauryl), tridecyl, tetradecyl (myristyl), pentadecyl, hexadecyl (palmityl, cetyl), heptadecyl, octadecyl (stearyl), nonadecyl, icosyl (arachidinyl), docosyl (behenyl). The portion providing improved hydrophobicity can be linked by any linker being known in prior art. Preferably, the fatty acid can be linked by a peptide bond to the N-terminal amino acid of the inventive peptide.

This modification can be achieved by methods known in the art for other pharmaceutical active peptides (see e.g. Araujo F., et al. Nanoscale (2016) 19, 8(20), 10706-13 (Doi: 10.1039/c6nr00294c); Bukrinski J T et al., Biochemistry (2017) Sep. 12, 56(36), 4860-4870, (doi: 10.1021/acs.biochem.7b00492); Te Welscher Y M et al. J Control Release (2014) 175, 72-8, (doi: 10.1016/j.jconre1.2013.12.013); Yang P Y et al. PNAS (2016), 113(15):4140-5, (10.1073/pnas.1601653113); Ahmad J. et al., Curr Pharm Des (2017), 23(11), 1575-88, (doi: 10.2174/1381612823666170124111142); Gonzalez-Paredes A. et al., Int J Pharm (2017) 529(1-2), 474-485 (doi: 10.1016/j.ijpharm.2017.07.001); He Z Y et al., Int J Pharm (2014) 469(1),168-78 (doi: 10.1016/j.ijpharm.2014.04.056).

It can be provided that the apoptotically active substance comprises a peptide chain being formed by the first peptide portion and the second and/or third peptide portion and the peptide chain preferably has a solubility in water of at most 1.95 g/I, preferably of at most 1.0 g/l.

It can be provided that the peptide chain being formed by the first peptide portion and the second and/or third peptide portion and the peptide chain preferably has a solubility in water at most 1.95 g/l, preferably of at most 1.0 g/l.

Methods and means for determining the solubilities of the compounds described herein are known in the art. Preferably, the solubilities of the compounds described herein are determined by methods and means accepted by the FDA and/or EMEA.

Solubility in this regard preferably refers to the saturation solubility, which is preferably the maximum mass of the respective compound, which can be solubilised or dissolved in a solvent at a respective temperature and at a specific pressure, preferably atmospheric pressure.

With regard to the present invention, the solubilities in water given herein for the respective compound preferably refer to the saturation solubility of the respective compound in water, which is preferably the maximum mass of the respective compound which can be solubilised or dissolved in water at the respective given temperature and at the respective pressure, preferably atmospheric pressure, and even more preferably the maximum mass of the respective compound which can be solubilised or dissolved in water at the respective temperatures given herein, i.e. 20° C. and/or 25° C., preferably 20° C., and at the respective pressure, preferably atmospheric pressure, which is here preferably normal atmospheric pressure and especially the standardised “normal” atmospheric pressure, i.e. 1 atm=1.01325 bar.

Even more preferably, they can be determined by the method described below:

10 mL of solvent is placed in an amber glass ampul and sufficient substance is added to yield a distinct sediment that remains on the bottom after mixing thoroughly. After standing for 15 minutes and mixing again the ampuls are sealed and shaken in a thermostatically controlled water bath (20° C./16 hours or 25° C./16 hours, preferably 20° C./16 hours). Afterwards the ampuls are opened and the supernatant solution is filtered until the filtrate is clear. The content of the substance is determined photometrically in an aliquot by means of the specific adsorption coefficient. The respective dilution of the solvent without substance serves as blank. The solubility is expressed in the dimension of g substance in 100 mL or mg substance in 1 mL, preferably in mg substance in 1 mL. Preferably, this method is performed at normal atmospheric pressure and especially at the standardised “normal” atmospheric pressure, i.e. 1 atm=1.01325 bar.

The lipophilicity of hexapeptides may also be increased, for example by attaching phenylalanines to the peptide sequence.

The peptides mentioned above may comprise cyclic parts and may comprise a N-terminal modification. Cyclization and N-terminal modification of peptides has been described, for example, by Borchard, Journal of controlled Release 62 (1999), 231-238, and by Blackwell et al., J. Org. Chem 10 (2001), 5291-302.

According to the invention, a cyclic peptide or cyclic oligopeptide is preferably a homodetic cyclic peptide or homodetic cyclic oligopeptide. The meaning of the terms “homodetic”, “homodetic cyclic peptide” and homodetic cyclic oligopeptide is known in the art. According to the invention, a homodetic cyclic peptide or homodetic cyclic oligopeptide preferably is a cyclic peptide in which the ring (or backbone of the cyclic peptide) consists solely of amino-acid residues in peptide linkage (or in eupeptide linkage according to the nomenclature of the IUPAC).

Cyclic peptides and also the starting materials for their preparation are preferably prepared by known methods, preferably as described in the literature (for example in the standard works such as Houben-Weyl, Methoden der organischen Chemie [Methods of Organic Chemistry], Georg-Thieme-Verlag, Stuttgart), in particular under reaction conditions which are known and appropriate for the said reactions. In this context, use can also be made of known variants which are not mentioned in any greater detail here.

It is therefore obvious that a person skilled in the art, proceeding from the knowledge imparted by the present invention, will easily arrive at a whole number of derived peptide mimetics, which, however, are all covered by the present invention.

The apoptotically active substance inducing apoptosis, preferably the peptides mentioned above may be used as a salt.

A base of a peptide can be converted into the associated acid addition salt using an acid. Suitable acids for this reaction are, in particular, those which yield physiologically acceptable salts. Thus inorganic acids can be used, examples being sulfuric acid, nitric acid, hydrohalic acids such as hydrochloric acid or hydrobromic acid, phosphoric acid such as orthophosphoric acid, sulfamic acid, and also organic acids, especially aliphatic, alicyclic, araliphatic, aromatic or heterocyclic mono- or polybasic carboxylic, sulfonic or sulfuric acids, for example formic acid, acetic acid, propionic acid, pivalic acid, diethyl-acetic acid, malonic acid, succinic acid, pimelic acid, fumaric acid, maleic acid, lactic acid, tartaric acid, malic acid, benzoic acid, salicylic acid, 2-or 3-phenylpropionic acid, citric acid, gluconic acid, ascorbic acid, nicotinic acid, isonicotinic acid, methane- or ethanesulfonic acid, ethanedisultonic acid, 2-hydroxyethanesulfonic acid, benzenesulfonic acid, p-toluenesulfonic acid, naphthalene-mono- and -disulfonic acids, laurylsulf uric acid. Salts with physiologically unacceptable acids, for example picrates, can be used for isolating and/or purifying the pharmaceutically active substance according to the present invention.

Alternatively, an acid of a peptide can be converted into one of its physiologically acceptable metal or ammonium salts by reaction with a base. Particularly suitable salts in this context are the sodium, potassium, magnesium, calcium and ammonium salts, and also substituted ammonium salts, for example the dimethyl-, diethyl- or diisopropylammonium salts. monoethanol-, diethanol- or triethanolammonium salts. cyclohexylammonium salts, dicyclohexylammonium salts, dibenzylethylene diammonium salts, and also, for example, salts with N-methyl-D-glucamine or with arginine or lysine.

The pharmaceutically active substance of the present invention is preferably employed as a pharmaceutically acceptable salt, more preferably the pharmacologically acceptable hydrochloride salt, and especially preferably applied as the inner (or internal) salt.

The new apoptotically active substance of the present invention can be used for the treatment of cancer. Therefore, the present invention provides a pharmaceutical composition, comprising at least one apoptotically active substance of the present invention or a pharmaceutically acceptable salt of this apoptotically active substance and a pharmaceutically acceptable carrier.

The invention also provides pharmaceutical compositions comprising an effective amount of an apoptotically active substance, preferably a peptide, a protein or peptide mimetic, in combination with a conventional pharmaceutical carrier.

For example, a pharmaceutical carrier is a solid or liquid filler, an encapsulation material or a solvent. Examples of materials that can serve as pharmaceutical carriers include sugars, such as lactose, glucose and sucrose; starch such as corn starch and potato starch, cellulose and the derivatives thereof, such as sodium carboxymethyl cellulose, ethyl cellulose and cellulose acetate; pulverized tragacanth; malt, gelatine, tallow; drug carriers such as cocoa butter and suppository wax; oils such as peanut oil, cottonseed oil, safflower oil, sesame oil, olive oil, corn oil and soy bean oil; polyalcohols such as propylene glycol, glycerol, sorbitol, mannitol and polyethylene glycol; esters such as ethyl oleate and ethyl laureate; agar, buffering agents such as magnesium hydroxide and aluminium hydroxide; alginic acid; pyrogen-free water; isotonic salt solution; Ringer's solution, ethyl alcohol and phosphate buffer solutions, as well as other non-toxic compatible substances used in pharmaceutical formulations. Wetting agents, emulsifiers and lubricants such as sodium laureth sulphate and magnesium stearate, as well as dyes, coating agents, perfuming agents and preservatives can also be present in the preparations, in keeping with the requirements of the formulation specialist. The amount of the active agent that is combined with the carrier materials to produce an individual dose will vary depending on the patient to be treated and the particular method of administration.

In a preferred embodiment, the pharmaceutically active substance can be used as a solution and/or suspension in a liquid pharmaceutical carrier. Preferably, water or a mixture comprising water as mentioned above and below can be used.

Substances according to the invention that are active components of a pharmaceutical preparation are preferably dissolved in a pharmaceutically acceptable carrier.

Examples of pharmaceutically acceptable carriers can be buffer solutions such as phosphate buffers or citrate buffers. So as to maintain the activity of the peptides, reagents that are pharmaceutically acceptable may also be added, maintaining, for example, a reducing environment in the pharmaceutical preparation.

It can be provided that the pharmaceutical compositions preferably comprises of a pharmaceutically active substance in a concentration in the range of from 0.5 to 80%, more preferably 1.5 to 60%, even more preferably 3 to 40%, more preferably from 4 to 30%, even more preferably from 5 to 20% by weight.

If not explicitly stated otherwise, the percentages (%) given with respect to the instant invention and especially the percentages (%) given with respect to the compositions according to the invention are preferably selected from

i) percent by weight (% by weight or % w/w),
ii) percent by volume (% by volume or % v/v), and
iii) percent weight by volume (% weight by volume or % w/v, e.g. % mg/mL or % g/mL).

For ease of use, percent by weight and percent weight by volume are preferred and percent weight by volume is especially preferred, especially with respect to the compositions according to the invention.

In a further embodiment, the pharmaceutically active substance can be used in solid state, e. g. as a particulate being formulated in an appropriate manner, e. g. as a suspension in a liquid pharmaceutical carrier. Preferably, water or a mixture comprising water or an oil or a mixture comprising an oil as mentioned above and below can be used.

In the case that the pharmaceutically active substance is used in solid state, the particles comprising the apoptotically active substance of the present invention preferably have a particle size particle size less than 250 μm, preferably less than 150 μm, more preferably less than 100 μm, even more preferably less than 50 μm.

Typically, the suspended or suspendable solid micro particles of the one or more oligopeptides contained in said compositions have a particle size of more than 0.001 μm, preferably more than 0.01 μm and especially more than 0.1 μm. However, even smaller particle sizes are preferably not critical for the compositions according to the invention. Preferably, the compositions as described herein preferably contain only minor amounts of suspended or suspendable solid micro particles of the one or more oligopeptides having a particle size of 0.01 μm or less, preferably 0.1 μm or less, and especially 1 μm or less. Minor amounts in this regard are preferably 10% or less, 5% or less, 1% or less, 0.1% or less, or 0.01% or less, based on the total amount of the one or more oligopeptides as described herein contained in said composition. Percentages in this regard are preferably % w/w.

Preferably, the particle size distributions of the suspended or suspendable solid micro particles of the one or more oligopeptides contained in said compositions are characterized by d(10)=1-10 μm, d(50)=10-25 μm and/or d(90)=25-60 μm, more preferably by d(10)=1-10 μm, d(50)=10-25 μm and d(90)=25-60 μm.

Alternatively preferably, the particle size distributions of the suspended or suspendable solid micro particles of the one or more oligopeptides contained in said compositions are characterized by d(10)=1-5 μm, d(50)=5-10 μm and/or d(90)=20-30 μm, more preferably by d(10)=1-5 μm, d(50)=5-10 μm and d(90)=20-30 μm.

Thus, especially preferred are compositions as described herein for use in the methods according to the invention, wherein the effective average particle size of the one or more oligopeptides contained in said compositions is in the range of 5 μm to 250 μm, preferably 5 μm to 150 μm, more preferably 10 μm to 250 μm, even more preferably 10 μm to 150 μm, even more preferably 10 μm to 100 μm and even more preferably 15 μm to 100 μm, and especially 20 μm to 100 μm.

Thus, especially preferred are compositions as described herein for use in the methods according to the invention, preferably characterized or additionally characterized by a particle size of the one or more oligopeptides contained in said compositions having a d(90) value in the range of 5 μm to 150 μm, preferably 5 μm to 100 μm, more preferably 10 μm to 100 μm, even more preferably 15 μm to 100 μm, even more preferably 25 μm to 100 μm and even more preferably 20 μm to 50 μm, for example a d(90) of about 15 μm, a d(90) of about 20 μm, a d(90) of about 25 μm, a d(90) of about 30 μm, a d(90) of about 35 μm, a d(90) of about 40 μm or a d(90) of about 50 μm.

The term “particle size” as used herein is known and understood in the art. Preferably, the particle size is determined on the basis of the weight average particle size, preferably as measured by conventional particle size measuring techniques well known to those skilled in the art. Such techniques preferably include, for example, sedimentation field flow fractionation, photon correlation spectroscopy, light scattering, and disk centrifugation.

The term “average particle size” as used herein is known and understood in the art. Preferably, the average particle size is selected from the weight-average particle size, the volume-weighted average particle size and the number-weighted average particle size.

Preferably, the particle size and/or the average particle size is measured by light-scattering methods, microscopy or other appropriate methods known in the art. Appropriate methods in this regard preferably include, but are not limited to sedimentation field flow fractionation, photon correlation spectroscopy, light scattering, laser dynamic light scattering, and disk centrifugation. Furthermore, dynamic light scattering methods (e.g., photocorrelation spectroscopy, laser diffraction, low angle laser light scattering (LALLS), medium-angle laser light scattering (MALLS), light obscuration methods (Courter method, for example), rheology, or microscopy (light or electron) can be used.

The determination of the particle size distribution is especially preferably performed by laser diffraction, preferably on a Malvern Mastersizer 2000, preferably using the wet modul Hydro 2000 S M. The evaluation model is preferably Universal (normal sensitivity), the dispersion medium is preferably saturated placebo solution, the stirrer speed is preferably about 2000 rpm, the obscuration is preferably 10-15%, the background measuring time is preferably about 7500 ms (milliseconds), and/or the measuring time is preferably preferably about 7500 ms.

The specific dosage and posology depends for each patient on a number of factors, including the activity of the specific compounds used, the patient's age, the body weight, general health, the gender, nutrition, time of the administration, way of administration, the excretion rate, the combination with other pharmaceuticals, and the severity of the individual disease for which the therapy is being applied. It is ascertained by a physician based on these factors.

Preferably, the apoptotically active substance inducing apoptosis, preferably a peptide and/or a protein of the present invention is provided in a therapeutically effective amount. A “therapeutically effective amount” of an apoptotically active substance inducing apoptosis, preferably a peptide and/or a protein of the present invention refers to an amount effective, at dosages and for periods of time necessary, to achieve the desired therapeutic result. Such therapeutically effective amount may vary according to factors such as the disease state, age, sex, and weight of the individual, and the ability of the pharmaceutically active substance of the present invention to elicit a desired response in the individual. A therapeutically effective amount is also one in which any toxic or detrimental effects of the apoptotically active substance inducing apoptosis, preferably a peptide and/or a protein of the present invention are outweighed by the therapeutically beneficial effects.

In the method according to the invention as described herein, the composition as described herein and/or the paragraphs relating thereto is preferably administered to the subject, preferably the mammalian subject and especially to the human subject in a manner that the amount of the pharmaceutically active substance according to the invention, preferably an oligopeptide and/or a protein, administered to said subject is 0.5 mg to 3000 mg per subject and day, more preferably 10 to 2500 mg per subject and per day, and especially 50 to 1000 mg per patient and per day, or, per kilogram body weight, preferably about 0.1 to 100 mg/kg, and more preferably 1 mg to 50 mg/kg, preferably per dosage unit and more preferably per day, or, per square meter of the body surface, preferably 0.5 mg to 2000 mg/m², more preferably 5 to 1500 mg/m², and especially 50 to 1000 mg/m², preferably per dosage unit and more preferably per day. Said amounts preferably relate on every day on which the formulation is administered to said subject. Said formulation is preferably suitable to be administered to said subject daily, i.e. once every day, or even two or three times daily, i.e. two times every day or three times every day, for a prolonged time period, i.e. several weeks to several years and more preferably 1 week to 2 or 3 years. Due to the advantageous pharmacokinetic profile of said formulation, said formulation is preferably also suitable to be administered to said subject less frequent, i.e. to times weekly, once weekly or every second week.

In the method according to the invention as described herein and/or the paragraphs relating thereto, the composition as described herein and/or the paragraphs relating thereto is preferably administered to the subject, preferably the pharmaceutically active substance according to the invention, preferably an oligopeptide and/or a protein administered to said subject is 2 mg to 9000 mg per subject and per week (weekly dose), more preferably 30 to 7500 mg per subject and per week (weekly dose), and especially 150 to 4500 mg per subject and per week (weekly dose), or, per kilogram body weight, preferably about 0.5 to 200 mg/kg per subject and per week (weekly dose), and more preferably 1 mg to 150 mg/kg per subject and per week (weekly dose), or, per square meter of the body surface, preferably 20 mg to 6000 mg/m² per subject and per week (weekly dose), more preferably 100 to 3000 mg/m², and especially 200 to 2000 mg/m² per subject and per week (weekly dose).

Generally, the apoptotically active substance inducing apoptosis, preferably the oligopeptide and/or the protein, and/or the pharmaceutically acceptable derivatives, solvates and/or salts thereof and/or the one or more cancer cotherapeutic agents or further cancer cotherapeutic agents, more preferably the one or more cancer chemotherapeutic agents, can be administered in an amount and/or a regimen as it is known in the art for the respective compound.

In the method according to the invention as described herein and/or the paragraphs relating thereto, the composition as described herein and/or the paragraphs relating thereto is preferably administered to the human subject in a manner that the amount of apoptotically active substance inducing apoptosis, preferably oligopeptide and/or protein and/or the pharmaceutically acceptable derivatives, solvates and/or salts thereof, administered to said subject is 50 mg to 3000 mg per subject and day, more preferably 100 to 2000 mg per subject and per day, even more preferably 100 to 1000 mg per subject and per day and especially 150 to 700 mg per patient and per day, or, per kilogram body weight, preferably about 1 to 60 mg/kg, and more preferably 2 mg to 30 mg/kg, preferably per dosage unit and more preferably per day, or, per square meter of the body surface, preferably 50 mg to 1000 mg/m², more preferably 50 to 500 mg/m², and especially 75 to 350 mg/m², preferably per dosage unit and more preferably per day. Said amounts preferably relate on every day on which the formulation is administered to said subject. Said formulation is preferably suitable to be administered to said subject daily, i.e. once every day, or even two or three times daily, i.e. two times every day or three times every day, for a prolonged time period, i.e. several weeks to several years and more preferably 1 week to 2 or 3 years. Due to the advantageous pharmacokinetic profile of said formulation, said formulation is preferably also suitable to be administered to said subject less frequent, i.e. to times weekly, once weekly or every second week.

In the method according to the invention as described herein and/or the paragraphs relating thereto, the composition as described herein and/or the paragraphs relating thereto is preferably administered to the human subject in a manner that the amount of apoptotically active substance inducing apoptosis, preferably oligopeptide and/or protein and/or the pharmaceutically acceptable derivatives, solvates and/or salts thereof, administered to said subject is 75 mg to 9000 mg per subject and per week (weekly dose), more preferably 150 to 5000 mg per subject and per week (weekly dose), even more preferably 300 to 4500 mg per subject and per week (weekly dose) and especially 600 to 2500 mg per subject and per week (weekly dose).

Said weekly dose is preferably administered to said subject for at least one week, preferably at least two weeks, more preferably at least four weeks and especially at least eight weeks, preferably without preferably without a pause or substantially without a pause. Preferably, due to the advantageous properties of said composition, the duration of said weekly administration is in principle not limited. Thus, said weekly dose is preferably administered to said subject for a time period 1 to 208 weeks, more preferably 2 to 156 weeks, even more preferably 4 to 156 weeks and especially 4 to 104 weeks or 4 to 52 weeks, preferably without a pause or substantially without a pause.

The pharmaceutical composition, preferably peptide pharmaceuticals are preferably administered parenterally, for example by way of an inhalation spray, rectally, by subcutaneous, intravenous, intramuscular, intra-articular and intrathecal injection and infusion techniques, or externally in pharmaceutical formulations containing conventional pharmaceutically acceptable carriers, adjuvants and vehicles. Preferably, the compositions are designated for subcutaneous (s.c.) administration and/or intramuscular (i.m.) administration. Depending on the type of the identified substance, other ways of administration, such as oral, can be considered as well. For skin cancer, the pharmaceuticals according to the invention are preferably administered in the form of ointments or powder.

Suitable routes, manners and devices for administering the compositions for use in the method according to the invention are known and described in the art.

Regarding oral administration, the pharmaceutical composition of the present invention, especially the pharmaceutically active substance of the invention, more preferably a peptide can be adopted and modified. In a specific embodiment, the pharmaceutically active substance of the invention, more preferably a peptide as mentioned above and below is modified by a glycosphingolipid, more preferably ceramide. In a preferred embodiment of this aspect of the present invention, the ceramide comprises (a) a short chain fatty acid (C4-C12) or (b) a long chain fatty acid (C14-C28) comprising at least one cis double bond between two carbon atoms and the agent to be delivered (the pharmaceutically active substance of the invention, more preferably a peptide as mentioned above and below) is attached to the oligosaccharide of the glycospingolipid.

A system comprising a modification by a glycosphingolipid this type is described and depicted in Reference WO 2010/027479 A2. Reference WO 2010/027479 A2 is hereby incorporated by reference.

Preferably, the compositions for use in the method according to the invention are administered to the subject parenterally.

Even more preferably, the compositions for use in the method according to invention are administered to the subject via an injection.

Even more preferably, the compositions for use in the method according to the invention are administered to the subject subcutaneously and/or intramuscular.

Especially preferably, the compositions for use in the method according to the invention are administered to the subject by subcutaneous and/or intramuscular injection, even more preferably by subcutaneous injection.

Suitable devices for administering said compositions to the subject are known in the art. Preferred according to the invention are syringes and/or other devices for injection of fluid compositions into the body of the subject. Suitable such devices are known and described in the art.

Especially preferred syringes and devices for administering said compositions at the subject, preferably a human subject, are syringes and devices that allow a self administration by said subject. Suitable such devices are known and described in the art.

A further preferred subject of the instant invention is a process for the manufacture of a composition as described herein.

Administration in this regard preferably relates to the administration of said compositions to a mammal, preferably a human mammal, even more preferably to a patient and especially to a human patient. In this regard, subcutaneous administration or subcutaneous is preferably also abbreviated as s.c. administration or s.c., respectively; also in this regard, intramuscular administration or intramuscular is preferably abbreviated as i.m. administration or i.m.

Pharmaceutically acceptable salts of the substances according to the invention, preferably peptides, proteins or peptide mimetics, can be produced in well-known ways, for example by dissolving the compounds according to the invention in the appropriate diluted acid or base, for example hydrochloric acid or sodium hydroxide, followed by freeze-drying. Metal salts can be obtained by dissolving the compounds according to the invention in solutions containing the appropriate ion, and subsequently isolating the compound by way of HPLC or gel permeation methods.

According to an embodiment of the present invention, it can be provided that pharmaceutical composition preferably comprises at least one further medicament active ingredient, preferably a cytostatic. The apoptotically active substance of the present invention can be used as single compound or as a mixture of two, three, four, five or more compounds according to the present invention. The term “further medicament active ingredient” implies that the “medicament active ingredient” additionally used is different from the apoptotically active substance of the present invention. Preferably, the medicament active ingredient is a cytostatic agent and/or a mixture comprising a cytostatic agent.

Cytostatic agents are well known in the art. Cytostatic agents or chemotherapeutic agents is a chemical compound useful in the treatment of cancer. Examples of cytostatic agents include alkylating agents such as thiotepa and cyclophosphamide;

alkyl sulfonates such as busulfan, improsulfan, and piposulfan; aziridines such as benzodopa, carboquone, meturedopa, and uredopa;

ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol); beta-lapachone; lapachol; colchicines; betulinic acid; a camptothecin (including the synthetic analogue topotecan (CPT-1 1 (irinotecan)), acetylcamptothecin, scopolectin, and 9-aminocamptothecin); bryostatin;

pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine;

antibiotics such as the enediyne antibiotics (e. g., calicheamicin, especially calicheamicin gammaII and calicheamicin omegall (see, e.g., Nicolaou et ah, Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino- doxorubicin, 2-pyrroline- doxorubicin, doxorubicin HCl liposome injection and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine, tegafur, capecitabine, an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); thiotepa; taxoids, e.g., paclitaxel, albumin-engineered nanoparticle formulation of paclitaxel, and doxetaxel; chloranbucil; 6-thioguanine; mercaptopurine; methotrexate; platinum analogs such as cisplatin and carboplatin; vinblastine; platinum; etoposide (VP-1 6); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin combined with 5-FU and leucovovin.

“Platinum-based chemotherapy” as used herein refers to therapy with one or more platinum-based chemotherapeutic agents, optionally in combination with one or more other chemotherapeutic agents.

According to an embodiment of the present invention, it can be provided that pharmaceutical composition is formulated being designed for controlled release.

In specific embodiments, these systems preferably comprise a polymer-linker-drug conjugate and a recognition segment. The recognition segment is preferably an oligopeptide and/or an oligosaccharide. Preferably, the polymer is a polymeric carrier, more preferably the polymeric carrier is hydrophilic, biocompatible and biodegradable. Preferably, the polymeric carrier is larger than the renal excretion limit. Biocompatible polymers are not significantly toxic to cells. In order to prevent chromic accumulation of polymeric carriers that are larger than the renal excretion limit are preferably both biocompatible and biodegradable. Biodegradable polymers are preferably broken down by the cellular machinery and/or by hydrolysis into components that the cells can either reuse or dispose of without significant toxic effect. In preferred embodiments, a biodegradable polymer and its biodegradation by products are biocompatible. It is to be understood that any known biodegradable polymer may be incorporated in a conjugate. Preferred polymeric carriers are hydrophilic, e.g., they may include polar groups, such as hydroxyl or amine groups; anionic groups, such as carboxylate, sulfonate, sulphate, phosphate, or nitrate groups; or cationic groups, such as protonated amine, quaternary ammonium, or phosphonium groups, Suitable hydrolytically degradable polymers known in the art include for example, certain polyesters, polyanhydrides, polyorthoesters, polyphosphazenes and polyphosphoesters. Other biodegradable polymers known in the art, include, for example, certain carbohydrates, polyhydroxyacids, polypropylfumerates, polycaprolactones, polyamides, poly(amino acids), polyacetals, polyethers, biodegradable polycyanoacrylates and biodegradable polyurethanes. For example, specific biodegradable polymers that may be used in the present invention include but are not limited to alginate, carboxymethyl-alginate, cellulose, polylysine, polylactic acid), poly(glycolic acid), poly(caprolactone), poly(lactide-co-glycolide), poly(lactide-co-caprolactone) and poly(glycolide-co-caprolactone). Those skilled in the art will recognize that this is an exemplary, not comprehensive, list of biodegradable polymers. It is further to be understood that inventive conjugates may comprise block co-polymers, graft co-polymers, or adducts of these and other polymers.

Preferably, the conjugates include a linker with at least a first and a second end. The linker preferably includes at least one segment that is cleaved when exposed to a digestive enzyme that is expressed in the target tissue. Preferably the digestive enzyme is overexpressed in the target tissue. In preferred embodiments the cleavable segment includes an oligopeptide or an oligosaccharide sequence. The first end of the linker is preferably associated with the polymeric carrier and the second end is preferably associated with the drug molecule. When two entities are “associated with” one another as described herein, they are linked by a covalent or ligand/receptor type interaction. Preferably, the association is covalent. One of ordinary skill in the art will appreciate that a whole host of synthetic methods exist for covalently linking oligopeptide or oligosaccharide segments with the polymeric carriers and drugs of the present invention.

The chemical composition of the cleavable segment (e.g., the length and sequence of amino acids or sugars) will depend for the most part on the motif that is recognized and cleaved by the digestive enzyme in the target tissue. The cleavage motifs are known for a number of proteases. For example, the table provided in Appendix A of document WO 2013/033717 A1 lists the cleavage motifs for a range of secreted or membrane bound proteases that are overexpressed in certain tumor tissues. These are all potential target enzymes that could be used to trigger release of drug molecules from inventive conjugates. The Examples describe the optimization of a cleavage segment that is recognized by matrix-metalloproteinase II (MMP-2), a proteinase that is also overexpressed in a variety of tumors.

A variety of methods are also known in the art that can be used to determine the cleavage motif of a target enzyme when it is not yet known. These include substrate phage display libraries (Matthews and Wells, Science 260:1113, 1993); positional-scanning peptide libraries (Rano et al., Chem. Biol. 4:149, 1996); and mixture-based peptide libraries (Turk et al., Nature Biotechnology 19:661, 2001). Positional-scanning synthetic peptide libraries are based on the detection of cleavage by the release of a C-terminal fluorogenic group. The technique is rapid and enables analysis of all possible peptide sequences. Currently the libraries only provide sequence specificity N-terminal to the cleavage site and cannot be used with proteases that require amino acid residues C-terminal to the cleavage site; this restricts their use largely to serine and cysteine proteases. Mixture-based peptide libraries offer an alternative that can provide both general applicability and speed. The cleavage site motif C-terminal to the cleavage site is first determined by partial digestion and N-terminal sequencing of a completely random peptide mixture. Information from this first round of screening is then used to design a second library in which strong selected amino acids are fixed, allowing data on sites N-terminal to the cleavage site to be obtained. Reiteration of this process allows an optimal recognition sequence to be determined. The method has recently been used to determine the cleavage motifs of a variety of matrix-metalloproteinases (Turk et al., Nature Biotechnology 19:661, 2001). The phage display method has been used to determine peptide substrates for a number of proteases, for example, plasmin (Hervio et al., Chemistry & Biology 7:443, 2000); tissue-type plasminogen activator (Ding et al., Proc. Natl.

Acad. Sci USA 92; 7627, 1995; Ke et al., J. Biol. Chem. 272; 16603, 1997); prostate-specific antigen (Coombs et al., Chemistry & Biology 5:475, 1998); and membrane type-1 matrix metalloproteinase (Ohkubo et al., Biochem. Biophys. Res. Commun. 266:308, 1999).

Preferably, sequences which are labile to target enzyme but resistant to serum proteins can be preferentially selected from a library of peptides. For example, in a first screen, phage that are released upon incubation with the target enzyme are enriched. These phage are then incubated with serum proteins. Phage which remain on the affinity support are then enriched. The DNA sequence of the phagemids can be determined and translated into an amino-acid sequence.

A system comprising a polymer-linker-drug conjugate and a recognition segment of this type is described and depicted in Reference US 2004/116348. Reference US 2004/116348 is hereby incorporated by reference. The peptides being disclosed as pharmaceutical active as mentioned in US 2004/116348 must be replaced by one or more pharmaceutically active substances, preferably apoptotically active substances inducing apoptosis of the present invention

In a further embodiment, particles encapsulating one or more pharmaceutically active substances, preferably apoptotically active substances inducing apoptosis of the present invention are preferably used for the applying the present drugs to a subject in need as mentioned above and below.

Preferably, the particles encapsulating one or more apoptotically pharmaceutically active substances, preferably apoptotically active substances inducing apoptosis of the present invention are preferably nanocapsules that can be tuned to release agents such as polypeptides into selected environments.

Preferably, polymeric nanocapsule are disposed around one or more polypeptides, wherein the nanocapsule is designed to degrade in certain environments thereby releasing the polypeptides. Preferably these methods include forming a mixture comprising a polypeptide, a plurality of polymerizable monomers; and a crosslinking agent that includes a plurality of amino acids linked by peptide bonds in a sequence or “motif that is recognized and cleaved by a protease. In certain embodiments of this aspect of the invention, the nanocapsule is designed for use with proteases that recognize amino acid sequences that are at least 5 amino acids in length such as KRVK (SEQ ID NO: 101), GGIPVSLRSGGK (SEQ ID NO: 102) or GGVPLSLYSGGK (SEQ ID NO: 103). In certain embodiments of this aspect of the invention, polymeric nanocapsules used in these methods are disposed within a matrix comprising a hydrogel. Optionally, the polymeric nanocapsules are covalently coupled to the hydrogel (e.g. via a crosslinking agent disclosed herein).

In methods to obtain these embodiments according to this aspect of the invention, the nanocapsule forming mixture is exposed to conditions that first allow the plurality of polymerizable monomers and the crosslinking agent to adsorb to surfaces of the polypeptide. Polymerization of the plurality of polymerizable monomers and the crosslinking agent at interfaces between the monomers and the polypeptide is then initiated so that a modifiable polymeric nanocapsule is formed, one that surrounds and protects the polypeptide. Typically, polymerization is initiated by adding a free radical initiator to the mixture and the resultant polymer chains are then linked together via one or more crosslinking agents. In illustrative embodiments, the crosslinking agent comprises a sequence of amino acids that are linked by peptide bonds, wherein the sequence comprises an amino acid motif that is recognized and cleaved by one or more proteases. In typical embodiments according to this aspect of the invention, the polypeptide is not covalently coupled to the polymeric nanocapsule following the polymerization of the plurality of polymerizable monomers and the crosslinking agent, and is free to migrate away from the nanocapsule upon loss of its integrity (e.g. as a result of cleavage of its peptide bonds). Optionally, the mixture comprises a plurality of polypeptides associated within a protein complex (e.g. a multimeric protein complex). In certain embodiments of the invention, polymeric nanocapsules used in these methods are formed within a matrix comprising a hydrogel (e.g. by controlling polymerization conditions).

Illustrative embodiments include methods of controlling the release of a polypeptide cargo from a polymeric nanocapsule into a selected environment (e.g. control the rate at which polypeptide is released and/or control/select the specific environment in which polypeptide is released). In these methods, enzyme-responsive protein nanocapsules are synthesized that can release protein cargoes in response to specific enzymes (e.g. proteases secreted during certain cellular events) with a high degree of specificity as well as a controlled rate of release (e.g. by controlling reaction condition so as to tune the material profiles of the nanocapsule compositions). Typically these polymeric nanocapsules are formed from a mixture comprising the polypeptide, polymerizable monomers, an initiator that reacts with the polymerizable monomers so as to generate polymers, wherein the polymers form the polymeric nanocapsule, a first crosslinking agent that links the polymers, wherein the first crosslinking agent is selected to comprise a peptide having an amino acid sequence that is cleaved by a protease, and a second crosslinking agent that links the polymers, wherein the second crosslinking agent does not comprise a peptide having an amino acid sequence that is cleaved by the protease. In certain embodiments of the invention, polymeric nanocapsules are disposed within a three dimensional matrix comprising a hydrogel.

In typical methods, the nanocapsule is designed so that protease mediated cleavage of the peptide in the first crosslinking agent degrades the polymeric nanocapsule so as to release the polypeptide from the polymeric nanocapsule and allow it to migrate in to the external environment. In these methods, the relative amounts of the first crosslinking agent and the second crosslinking agent in the mixture can be selected to control release of the polypeptide from the polymeric nanocapsule. Further methodological steps in this embodiment of the invention comprise placing the polymeric nanocapsules in an environment selected to include a protease that cleaves the amino acid sequence of the peptide, and then allowing the protease in the selected environment to cleave the amino acid sequence of the peptide, thereby releasing the polypeptide from the polymeric nanocapsule into the selected environment. In certain embodiments of the invention, the protease is produced by a human cell within the selected environment (e.g. an in vivo environment).

Another embodiment of the invention is a method of forming a polymeric nanocapsule around a polypeptide, wherein the polymeric nanocapsule is designed to release the polypeptide into a selected environment. These methods comprise forming a mixture that includes the polypeptide of interest (preferably a pharmaceutically active substance according to the present invention), a plurality of polymerizable monomers, a first crosslinking agent comprising a peptide having an amino acid sequence that is cleaved by a protease and a second crosslinking agent does not comprise a peptide having an amino acid sequence that is cleaved by the protease. These methods further comprise allowing the plurality of polymerizable monomers and the crosslinking agents to adsorb to surfaces of the polypeptide and then initiating polymerization of the plurality of polymerizable monomers and the first and second crosslinking agents at interfaces between the monomers and the polypeptide. In this way, proteolytically degradable polymeric nanocapsules are formed around one or more polypeptides. In certain embodiments of the invention, the polymeric nanocapsule is formed from a mixture further comprising a third crosslinking agent that links a first polymer with a second polymer, wherein the third crosslinking agent comprises a peptide having an amino acid sequence that is cleaved by a protease. As discussed herein, embodiments of the invention can be adapted for use with a wide variety of proteases. In illustrative embodiments of the invention, amino acid sequence that is cleaved by the protease comprises a sequence cleaved by plasmin such as KRVK (SEQ ID NO: 101), or a sequence cleaved by matrix metalloproteinase such as GGIPVSLRSGGK (SEQ ID NO: 102) or GGVPLSLYSGGK (SEQ ID NO: 103).

Yet another embodiment is a composition of matter comprising a constellation of elements that are arranged in the composition to form proteolytically degradable polymeric nanocapsules. Typically these compositions include at least one polypeptide and a polymeric network, wherein polymers in the polymeric network are coupled together by a first crosslinking agent and a second crosslinking agent so as to form a shell that encapsulates the polypeptide. Optionally in these embodiments, the shell that encapsulates the polypeptide has a diameter between 15 and 35 nanometers. Preferably, the first crosslinking agent comprises a peptide having an amino acid sequence that is cleaved by a protease, and the second crosslinking agent does not comprise a peptide having an amino acid sequence that is cleaved by the protease. In such compositions, the polymers, the first crosslinking agent and the second crosslinking agent are disposed within the polymeric network in an orientation so that proteolytic cleavage of the first crosslinking agent releases the polypeptide from the shell into an external environment. A wide variety of polypeptides can form the cargo of the polymeric nanocapsules disclosed herein. Preferably, the polypeptide is an apoptotically active substance inducing apoptosis

A system comprising a particle encapsulating peptides of this type is described and depicted in Reference WO 2013/033717 A1. Reference WO 2013/033717 A1 is hereby incorporated by reference. The peptides being disclosed as pharmaceutical active as mentioned in WO 2013/033717 A1 must be replaced by one or more pharmaceutically active substances, preferably apoptotically active substances inducing apoptosis of the present invention.

A preferred subject of the instant invention is the use of apoptotically active substance inducing apoptosis, the pharmaceutically acceptable derivatives, solvates and/or salts thereof, for the manufacture of a composition as described herein and/or the paragraphs relating thereto that is administered to a subject in a method for treating disorders, preferably disorders as described herein and and/or the paragraphs relating thereto.

An especially preferred subject of the instant invention is the use of the apoptotically active substance inducing apoptosis, the pharmaceutically acceptable derivatives, solvates and/or salts thereof, preferably the preferred embodiments thereof as mentioned above and below, for the manufacture of a composition for the treatment of disorders, wherein the composition is as described herein and/or the paragraphs relating thereto, and preferably wherein disorders to be treated are as described herein and/or the paragraphs relating thereto.

Another preferred subject of the invention relates to the use of the composition as described herein, as a pharmaceutical for treating disorders, preferably disorders as described herein.

The term “disorders” is known and understood in the art. Preferably, the disorders to be treated with the composition according to the invention are hyperproliferative disorders, more preferably oncologic disorders and especially cancerous disorders.

Preferably, the disorders to be treated are selected from cancer and metastases thereof.

A further subject matter of the present invention is a use of an apoptotically active substance of the present invention or of a pharmaceutically acceptable salt thereof for preparation of a drug. Preferably the apoptotically active substance of the present invention or of a pharmaceutically acceptable salt thereof is used for preparation of a drug for the treatment of cancer disease. Especially preferred is a method for treating disorders, especially disorder selected from cancer and/or metastases thereof, preferably cancer and/or metastases thereof as described herein.

A further subject matter of the present invention is a drug for the treatment of cancer, comprising at least one apoptotically active substance of the present invention or a pharmaceutically acceptable salt of the apoptotically active substance and a pharmaceutically acceptable carrier.

Preferably, the cancer disease is related to cancer of head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, brain, central nervous system, solid tumors and blood-borne tumors.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

The drug of the present invention and/or the pharmaceutical composition is preferably used for the treatment of advanced cancer, recurrent cancer, unresectable cancer, metastatic cancer and/or locally advanced cancer.

“Advanced” cancer is one which has spread outside the site or organ of origin, either by local invasion or metastasis. Accordingly, the term “advanced” cancer includes both locally advanced and metastatic disease.

“Recurrent” cancer is one which has regrown, either at the initial site or at a distant site, after a response to initial therapy, such as surgery. A “locally recurrent” cancer is cancer that returns after treatment in the same place as a previously treated cancer.

“Unresectable” cancer is not able to be removed (resected) by surgery.

“Metastatic” cancer refers to cancer which has spread from one part of the body (e.g. the lung) to another part of the body.

“Locally advanced” cancer refers to cancer that has spread to nearby tissues or lymph nodes, but not metastasized. “Advanced unresectable” cancer is one which has spread outside the site or organ of origin, either by local invasion or metastasis and which is not able to be removed (resected) by surgery.

“Subject” includes a human patient. The patient may be a “cancer patient,” i.e. one who is suffering or at risk for suffering from one or more symptoms of cancer, in particular non-small cell lung cancer. “Infusion” or “infusing” refers to the introduction of a drug-containing solution into the body through a vein for therapeutic purposes. Generally, this is achieved via an intravenous (IV) bag. “Systemic treatment” is a treatment wherein the drug substance travels through the bloodstream, reaching and affecting cells all over the body.

Preferably, the cancers to be treated are selected from solid tumours and/or metastases thereof.

The terms “hyperproliferative disorders”, “oncologic disorders”, “cancer”, “solid tumours” and “metastases” are known and understood in the art.

The terms “cancer” and/or “tumor” preferably refer to or describe the physiological condition in subjects, preferably mammalian subjects and even more preferably humans, that is typically characterized by upregulated or preferably unregulated cell growth, even more preferably benign and/or malignant cell growth and especially malignant cell growth. Especially preferably, the term “cancer” as used herein includes malignant neoplasms or consists of malignant neoplasms.

Typically, the terms “cancer” or “malignant neoplasms” describe a class of diseases in which a group of cells display uncontrolled growth, invasion that intrudes upon and destroys adjacent tissues, and sometimes metastasis, or spreading to other locations in the body via lymph or blood.

The term “metastases” (singular: metastasis) is known and understood in the art.

In the context of the present invention metastasis or metastatic disease (sometimes abbreviated mets), preferably refers to the spread of a cancerous disease from one organ or part to another organ or part, preferably a non-adjacent organ or part. The word metastasis means “displacement” in Greek. The plural is metastases.

According to an established theory, cancer occurs after a single cell in a tissue is progressively genetically damaged to produce a cancer stem cell possessing a malignant phenotype. These cancer stem cells are thought to be able to undergo uncontrolled abnormal mitosis, which would then serve to increase the total number of cancer cells at that location. When the area of cancer cells at the originating site become clinically detectable, it is preferably called primary tumor. Some cancer cells are also thought to acquire the ability to penetrate and infiltrate surrounding normal tissues in the local area, forming a new tumor. The newly formed “daughter” tumor in the adjacent site within the tissue is preferably called a local metastasis.

Some cancer cells are thought to be able to acquire the ability to penetrate the walls of lymphatic and/or blood vessels, after which they would then be able to circulate through the bloodstream (circulating tumor cells) to other sites and tissues in the body. This process is preferably known as lymphatic or hematogeneous spread, respectively.

After the tumor cells come to rest at another site, they seem to be able to re-penetrate through the vessel or walls, continue to multiply, and eventually another clinically detectable tumor is formed. This new tumor is known as a metastatic (or secondary) tumor. Metastasis is one of three hallmarks of malignancy (contrast benign tumors). Most tumors or malignant neoplasms can metastasize, although in varying degrees.

When tumor cells metastasize, the new tumor is preferably called a secondary or metastatic tumor, and its cells are like those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

Cancer cells may spread to lymph nodes (regional lymph nodes) near the primary tumor. This is called nodal involvement, positive nodes, or regional disease. (“Positive nodes” is a term that would be used by medical specialists to describe a patient's condition, meaning that the patient's lymph nodes near the primary tumor tested positive for malignancy. It is common medical practice to test by biopsy at least two lymph nodes near a tumor site when doing surgery to examine or remove a tumor.) Localized spread to regional lymph nodes near the primary tumor is preferably not counted as metastasis, although this is a sign of worse prognosis. Transport through lymphatics is the most common pathway for the initial dissemination of cancers or carcinomas.

There is a propensity for certain tumors to seed in particular organs. For example, prostate cancer usually metastasizes to the bones. In a similar manner, colon cancer has a tendency to metastasize to the liver. It is believed that it is difficult for cancer cells to survive outside their region of origin, so in order to metastasize they must find a location with similar characteristics. For example, breast tumor cells, which gather calcium ions from breast milk, metastasize to bone tissue, where they can gather calcium ions from bone. Malignant melanoma spreads to the brain, presumably because neural tissue and melanocytes arise from the same cell line in the embryo.

It is theorized that metastasis always coincides with a primary cancer, and, as such, is a tumor that started from a cancer cell or cells in another part of the body. However, over 10% of patients presenting to oncology units will have metastases without a primary tumor found. In these cases, doctors refer to the primary tumor as “unknown” or “occult,” and the patient is said to have cancer of unknown primary origin (CUP) or Unknown Primary Tumors (UPT). However, the use of immunohistochemistry has permitted pathologists to give an identity to many of these metastases. However, imaging of the indicated area only occasionally reveals a primary. In rare cases (e.g., of melanoma), no primary tumor is found, even on autopsy. It is therefore thought that some primary tumors can regress completely, but leave their metastases behind. Despite the use of various techniques, in some cases the primary tumor remains unidentified.

The formation of metastasis or metastases (via the metastatic process) is deemed to be a multistep event and represents the most dreadful aspect of cancer. At the moment of diagnosis, cancers are frequently far advanced in their natural history, and the presence of metastases is a common event. In fact, approximately 30% of patients have detectable metastases at the moment of clinical diagnosis and a further 30% of patients have occult metastases. Metastases can be disseminated and they can infest different organs at the same time, or localize to a specific organ. In the case of localized disease, surgery is the treatment of choice; however recurrence and prognosis depend on many criteria such as: resectability, patient's clinical situation, and number of metastases.

After resection, recurrence is common, suggesting that micrometastatic foci are present at the moment of diagnosis. Systemic chemotherapy is an ideal setting but only few patients are cured by it, and in the majority systemic chemotherapy fails. Many physiological barriers and pharmacokinetic parameters contribute to decrease its efficacy.

Liver, lungs and lymph nodes are filtration organs and therefore inclined to metastasization. The poor chemosensitivity of metastases, peculiarly those of colorectal origin has forced many researchers to use methods for increasing the time and the concentration of drugs. The need for decreasing or limiting the side effects for this important and delicate organ led to the development of the technique of liver isolation for perfusion of antineoplastic agents. (K. R. Aigner, Isolated liver perfusion. In: Morris D L, McArdle C S, Onik G M, eds. Hepatic Metastases. Oxford: Butterworth Heinemann, 1996. 101-107). Since 1981, modifications and technical improvements have been continuously introduced. Liver metastases may be of different origin and their chemosensitivity may vary according to the histological type and their response in presence of heat.

The terms cancer, breast cancer, lung cancer, head and neck cancer, prostate cancer, brain cancer, colorectal cancer, liver cancer, pancreatic cancer and malignant melanoma are known and understood in the art.

Prostate cancer is a form of cancer that develops in the prostate, a gland in the male reproductive system. Most prostate cancers are slow growing; however, there are cases of aggressive prostate cancers. The cancer cells may metastasize from the prostate to other parts of the body, particularly the bones and lymph nodes. The term prostate cancer preferably includes non-metastatic or metastatic prostate cancer. Even more preferably, the term prostate cancer includes androgen independent prostate cancer (AIPCa), androgen dependent prostate cancer (ADPCa), metastatic androgen independent prostate cancer and/or metastatic androgen dependent prostate cancer.

Colorectal cancer, less formally known as bowel cancer, is a cancer characterized by neoplasia in the colon, rectum, or vermiform appendix.

Liver cancer or hepatic cancer is properly considered to be a cancer which starts in the liver, as opposed to a cancer which originates in another organ and migrates to the liver, known as a liver metastasis. There are many forms of liver cancer, although many cancers found in the liver are metastases from other tumors, frequently of the GI tract (like colon cancer, carcinoid tumors mainly of the appendix, etc.), but also from breast cancer, ovarian cancer, lung cancer, renal cancer, pancreatic cancer, prostate cancer, etc. The most frequent liver cancer is hepatocellular carcinoma (HCC). This tumor also has a variant type that consists of both HCC and cholangiocarcinoma components. The cells of the bile duct coexist next to the bile ducts that drain the bile produced by the hepatocytes of the liver. Cancers which arise from the blood vessel cells in the liver are known has hemangioendotheliomas.

Pancreatic cancer is considered to be a cancer starting in the pancreas. There are a number of types of pancreatic cancer including pancreatic adenocarcinoma and non-adenocarcinoma. Pancreatic adenocarcinoma typically has a very poor prognosis. The present invention surprisingly provides a high effective drug against pancreatic cancer.

Cancer of the pancreas represents about 3% of newly diagnosed cancers, with the dominating subtype pancreatic ductal adenocarcinoma (PDAC) (Hezel et al., (2006) “Genetics and biology of pancreatic ductal adenocarcinoma.” Genes Dev 20(10): 1218-1249; Siegel et al., (2019) “Cancer statistics, 2019.” CA Cancer J Clin 69(1): 7-34). At the time of first diagnosis, only 10 to 20% of PDACs is resectable (Bilimoria et al. (2007) “National failure to operate on early stage pancreatic cancer.” Ann Surci 246(2): 173-180; Gillen et al., (2010) “Preoperative/neoadjuvant therapy in pancreatic cancer: a systematic review and meta-analysis of response and resection percentages.” PLoS Med 7(4): e1000267).

Pancreatic cancer is associated with a very poor prognosis, it has the lowest 5 year survival rate (8-9%) when considering the average of all tumor stages. PDAC is the fourth leading cause of cancer death in the United States (Siegel et al., (2016) “Cancer statistics, 2016.” CA Cancer J Clin 66(1): 7-30; Siegel et al., (2019) “Cancer statistics, 2019.” CA Cancer J Clin 69(1): 7-34).

The most common genetic alterations in PDAC are found in KRAS, CDKN2A, SMAD4 and TP53 (Bailey et al. (2016) “Genomic analyses identify molecular subtypes of pancreatic cancer.” Nature 531(7592): 47-52; Hezel et al. 2006). In addition, a number of other mutated genes including oncogenes BRAF and RET may drive tumor development (Grinshpun et al., (2019) “Beyond KRAS: Practical Molecular Targets in Pancreatic Adenocarcinoma.” Case Rep Oncol 12(1): 7-13).

At the time of diagnosis, more than half of the PDAC patients have metastases which are the major cause of death (Padoan et al., (2019) “Inflammation and Pancreatic Cancer: Focus on Metabolism, Cytokines, and Immunity.” Int J Mol Sci 20(3); Werner et al., 2013). Following a successful surgical resection of the tumor, the majority of patients show recurrence of metastasis (Mayo et al., (2012) “Conditional survival in patients with pancreatic ductal adenocarcinoma resected with curative intent.” Cancer 118(10): 2674-2681; Neoptolemos et al., (2004) “A randomized trial of chemoradiotherapy and chemotherapy after resection of pancreatic cancer.” N Engl J Med 350(12): 1200-1210).

Early stage pancreatic cancer may be treated by surgery plus adjuvant chemotherapy using either 5-fluorouracil (5-FU) or gemcitabine (both: inhibitors of DNA synthesis) (Herreros-Villanueva et al., (2012) “Adjuvant and neoadjuvant treatment in pancreatic cancer.” World J Gastroenterol 18(14): 1565-1572). Patients who had initially non-resectable tumors (borderline resectable/unresectable) and underwent a neoadjuvant combination chemotherapy may increase the chance of resection and possibly improve survival outcome (Gillen et al., 2010). In the process of pancreatic resections, dissection of regional lymph nodes and of lymph nodes along the main arteries is included (Werner et al., 2013)

A number of different single and combined chemotherapies have been applied and, in addition, chemotherapy has been combined with radiotherapy, named chemoradiation (Herreros-Villanueva et al., 2012; Werner et al., 2013). A failure to respond to chemotherapy which includes resistance to gemcitabine, 5-FU and/or cisplatin correlates with the rate of 63 genetic mutations on average in advanced pancreatic cancer, which may affect 12 different signaling pathways in the tumor cell (Jones et al., (2008) “Core Signaling Pathways in Human Pancreatic Cancers Revealed by Global Genomic Analyses.” Science 321(5897): 1801-1806). The nucleoside transporter hENT-1 is a predictive biomarker candidate for gemcitabine response since its over-expression appears to correlate with overall and disease-free survival of pancreas adenocarcinoma patients under gemcitabine treatment (Greenhalf et al., (2014) “Pancreatic cancer hENT1 expression and survival from gemcitabine in patients from the ESPAC-3 trial.” J Natl Cancer Inst 106(1): djt347; Farrell et al., (2009) “Human Equilibrative Nucleoside Transporter 1 Levels Predict Response to Gemcitabine in Patients With Pancreatic Cancer.” Gastroenterology 136(1): 187-195).

The combination therapy regime named FOLFIRINOX which contains folinic acid (leukovorin), 5-FU, irinotecan and oxaliplatin has been applied as an alternative to gemcitabine treatment of PDAC. FOLFIRINOX treatment resulted in a prolonged survival time, however, it caused more toxic effects including neurotoxicity compared to gemcitabine (Conroy et al., (2011) “FOLFIRINOX versus gemcitabine for metastatic pancreatic cancer.” N Engl J Med 364(19): 1817-1825; Vaccaro et al., (2015) “Metastatic pancreatic cancer: Is there a light at the end of the tunnel?” World J Gastroenterol 21(16): 4788-4801). The efficiency of FOLFIRINOX treatment depended on the expression of CES2, a carboxyl esterase which is believed to activate irinotecan in the tumor and therefore could serve as a predictive biomarker for therapy (Capello et al., (2015) “Carboxylesterase 2 as a Determinant of Response to Irinotecan and Neoadjuvant FOLFIRINOX Therapy in Pancreatic Ductal Adenocarcinoma.” J Natl Cancer Inst 107(8)).

A new formulation of the cancer drug paclitaxel not requiring a solvent, Nab-paclitaxel, consists of a nanoparticle with the protein albumin as a carrier. A combination therapy of Nab-paclitaxel with gemcitabine resulted in a prolonged survival time of 8.7 month compared to 6.6 month with gemcitabine alone (Goldstein et al., (2015) “nab-Paclitaxel plus gemcitabine for metastatic pancreatic cancer: long-term survival from a phase III trial.” J Natl Cancer Inst 107(2); Von Hoff et al., (2013) “Increased survival in pancreatic cancer with nab-paclitaxel plus gemcitabine.” N Engl J Med 369(18): 1691-1703).

A combination of chemotherapy, using 5-FU, gemcitabine and cisplatin, alone or in combination, with radiotherapy (chemoradiotherapy) may bring a benefit for the therapy of borderline resectable or unresectable diseases, where the treatment regime starts before resection (neoadjuvant chemoradiation) (Gillen et al., 2010; Regine et al., 2008; Werner et al., (2013) “Advanced-stage pancreatic cancer: therapy options.” Nature Reviews Clinical Oncology 10: 323).

A number of treatment studies for unresectable advanced pancreatic cancer were compared in a meta analysis (Chen et al., (2019) “Melanoma cell adhesion molecule is the driving force behind the dissemination of melanoma upon S100A8/A9 binding in the original skin lesion.” Cancer Lett) where gemcitabine, as the standard treatment, was applied together with one additional treatment, a chemotherapeutic drug or an antibody; or a combination of gemcitabine with a second drug plus an antibody (cetuximab, anti-EGF receptor antibody; bevacizumab, antibody directed against VEGF). The chemotherapeutic drugs combined with gemcitabine include 5-FU (DNA synthesis inhibitor), cisplatin (crosslinks DNA, blocks cell division), capecitabin (is converted to 5-FU), axitinib (tyrosine kinase inhibitor), erlotinib (EGF receptor tyrosine kinase inhibitor), etanercept (inhibitor of TNF alpha), exatecan (topoisomerase inhibitor), irinotecan (topoisomerase inhibitor), marimastat (matrix metalloproteinase inhibitor), nab-paclitaxel (blocks cell division), pemetrexed (folate anti-metabolite), sorafenib (inhibitor of tyrosine- and Raf family kinases), tipifarnib (farnesyltransferase inhibitor), vismodegib (smoothened receptor inhibitor); these treatments were compared with gemcitabine alone; or with gemcitabine combined with radiotherapy; or with the combination therapy FOLFIRINOX. Five treatments showed the highest activity for overall survival; five combinations were most effective in progression-free survival; and the following four treatments were effective with both criteria: gemcitabine plus radiotherapy, gemcitabine plus erlotinib plus bevacizumab, gemcitabine plus cisplatin, and gemcitabine plus capecitabin plus erlotinib (Chen et al., 2019).

Malignant melanoma is a malignant tumor of melanocytes. Melanocytes are cells that produce the dark pigment, melanin, which is responsible for the color of skin. They predominantly occur in skin, but are also found in other parts of the body, including the bowel and the eye (see uveal melanoma). Melanoma can occur in any part of the body that contains melanocytes. Melanoma is less common than other skin cancers. However, it is much more dangerous and causes the majority (75%) of deaths related to skin cancer. Worldwide, doctors diagnose about 160,000 new cases of melanoma yearly.

Cutaneous melanoma represents 1.6% of the newly diagnosed primary malignant tumors worldwide (Bray et al., (2018) “Global cancer statistics 2018: GLOBOCAN estimates of incidence and mortality worldwide for 36 cancers in 185 countries.” CA Cancer J Clin 68(6): 394-424). The risk to develop a melanoma is higher in the United States of America, Australia and Northern Europe compared to most other countries, new melanoma cases per year in the United States were estimated to 76,380 (Siegel et al., 2016).

The risk for melanoma is influenced by genetic factors but is induced by ultraviolet (UV) light exposure (Caini et al., (2009) “Meta-analysis of risk factors for cutaneous melanoma according to anatomical site and clinico-pathological variant.” Eur J Cancer 45(17): 3054-3063; Gandini et al., (2005a) “Meta-analysis of risk factors for cutaneous melanoma: II. Sun exposure.” Eur J Cancer 41(1): 45-60; Gandini et al., (2005b) “Meta-analysis of risk factors for cutaneous melanoma: Ill. Family history, actinic damage and phenotypic factors.” Eur J Cancer 41(14): 2040-2059), either due to sunlight exposure or to UV-A emitting tanning-beds. Spreading of the primary melanoma resulting in metastasis results in a 10 year survival rate of less than 10% (Balch et al., (2001) “Final version of the American Joint Committee on Cancer staging system for cutaneous melanoma.” J Clin Oncol 19(16): 3635-3648). The process of metastasis is incompletely understood, and recently it was shown to require the interaction of a specific cell adhesion molecule with its receptor and downstream signaling (Chen et al., 2019).

The number of mutations in melanoma is high compared to other cancers, with 100 mutations per 1 million base pairs in the tumor cell (Lawrence et al., (2013) “Mutational heterogeneity in cancer and the search for new cancer-associated genes.” Nature 499(7457): 214-218). Frequently mutated genes in melanoma are BRAF and NRAS (Curtin et al., (2005) “Distinct sets of genetic alterations in melanoma.” N Engl J Med 353(20): 2135-2147). In addition, a number of mutated genes are considered as tumor-driving (“driver”) mutants, such as TERT, PTEN, TP53, CDKN2A, PREX2, EZH2, IDH1 HRAS, RAC1 and NF1 (Hodis et al., 2012; Reddy et al., (2017) “Somatic driver mutations in melanoma.” Cancer 123(S11): 2104-2117). Other genes mutated during tumor development include KIT, GNA11, GNAQ, CTNNB1, PIK3CA and WT1 (Goldinger et al., (2013) “Targeted therapy in melanoma—the role of BRAF, RAS and KIT mutations.” EJC Supμl 11(2): 92-96; Hodis et al., 2012). In a 36 month follow up analysis of malignant melanomas, 21 out of 34 tumors developed nodal metastatic disease and showed changes in their gene expression including cell cycle, apoptosis, signal transduction and epithelial-to-mesenchymal transition (EMT), while in a second group of melanomas, the expression changes of certain EMT genes correlated with the development of metastasis (Alonso et al., (2007) “A high-throughput study in melanoma identifies epithelial-mesenchymal transition as a major determinant of metastasis.” Cancer Res 67(7): 3450-3460).

The surgical removal of malignant melanomas detected at an early stage as a localized disease can lead to over 98.4% of 5 year survival, while spreading to local or distal lymph nodes decreases the survival rate to 62.4 or 17.9%, respectively (National Cancer Institute, 2016, Cancer Stat Facts: Melanoma of the Skin).

Dacarbazine (DTIC) has been the only cytotoxic chemotherapy approved by the United States FDA for treatment of metastatic melanoma. From a pooled analysis of 23 controlled studies, 15.3% of the melanoma patients receiving dacarbazine showed an objective response (Lui et al., (2007) “Treatments for metastatic melanoma: synthesis of evidence from randomized trials.” Cancer Treat Rev 33(8): 665-680). Whether by this treatment (intravenous injection of dacarbazine) there is a survival benefit, however, has not been demonstrated (Bhatia et al., (2009) “Treatment of metastatic melanoma: an overview.” Oncology (Williston Park) 23(6): 488-496).

Temozolomide, an analog of dacarbazine which is administered orally has the advantage to penetrate into the brain and therefore might be used to treat or prevent brain metastases (Bhatia et al., 2009). In a clinical study comparing dacarbazine and temozolomide there was no significant difference between the two treatments in both the objective response rate and overall survival (Danson & Middleton, (2001) “Temozolomide: a novel oral alkylating agent.” Expert Rev Anticancer Ther 1(1): β-19).

In phase II or phase III clinical studies, various combination chemotherapies, compared with dacarbazine or temozolomide alone, showed increased response rates but no significant extension of survival, and greater toxicity (Bhatia et al., 2009).

The cytokine IL-2 stimulates the growth and function of T cells and of natural killer (NK) cells. Among a total of 270 patients treated with high-dose bolus IL-2, the objective response rate was 16%, with 6% complete and 10% partial response (Atkins et al., (1999) “High-dose recombinant interleukin 2 therapy for patients with metastatic melanoma: analysis of 270 patients treated between 1985 and 1993.” J Clin Oncol 17(7): 2105-2116). About half of the complete responders were long term responders, with 44% of responders surviving over 5 years (Atkins et al., 1999).

Interferon alpha 2b (pegylated; IFNalpha2b) is used in adjuvant therapy of resected high-risk melanoma. A meta analysis revealed that, compared to treatment without interferon, IFNalpha2b treatment resulted in a benefit in five years event-free survival (EFS) of 3.5%, and overall survival (OS) of 3.0% (Ives et al., 2017). This remarkable but weak effect, still leading to only 38% survivors at five years and 31% at ten years, contrasts with severe side effects of IFNalpha2b, especially at high doses. Both IL-2 and IFNalpha2b have been tested in combination with cytotoxic drugs (biochemotherapy) in a number of studies but did not lead to an improvement in overall survival (Bhatia et al., 2009).

Mutations in the BRAF gene at codon valine 600 (V600) are found in 66% of malignant melanomas. The protein kinase BRAF is located at the top of a kinase cascade which stimulates cell proliferation. The development of BRAF kinase inhibitors such as vemurafenib and dabrafenib could serve as a major breakthrough in melanoma therapy. In a phase III clinical study, overall survival of 13.6 months was seen in the vemurafenib group compared to 9.7 months in the dacarbazine group (Singh et al., 2016).

The blockade of immune inhibitory molecules such as CTLA-4 or PD-1 and its ligand PD-L1 by specific antibodies appears as an attractive new approach to improve the immune response against cancers including melanoma. Clinical phase I and phase II studies have indicated that blocking CTLA-4 or PD-1/PD-L1 has distinct effects, with clear improvements in objective response rate and overall survival of melanoma patients (Singh et al., 2016).

Lymphomas are derived from B lymphocytes (B cells), or from T lymphocytes or NK cells. The systematic classification divides lymphomas into two groups, Hodgkin lymphomas and non-Hodgkin lymphomas (NHL). The non-Hodgkin lymphomas, originating from B cells (in 85% of cases) which account for about 4% of all cancers in the Unites States, mostly derive from B lymphocytes and constitute a complex group of more than 30 diseases. B cell-derived lymphomas originate from defined states of B cell development in the secondary lymphatic organs which starts from naïve B cells and results in memory B cells or in antibody-producing plasma cells, respectively (Seifert et al., 2019). Epstein-Barr virus (EBV), a common human Herpes virus with an infection preference for mucosal epithelial cells and B cells, is associated with the development of lymphoproliferative diseases and lymphomas such as Burkitt lymphoma (Shannon-Lowe et al., 2017).

Chronic lymphocytic lymphomas (CLL) may derive from early state B cells (before somatic mutation) or from memory B cells (later stage); Burkitt lymphoma and Follicular lymphoma both develop from early stage, before somatic mutation of B cells; Mantle cell lymphomas derive from mantle cells; and lymphoplasmacytic lymphoma stems from plasma blasts, cells in late phase of B cell development (Seifert et al., 2019).

Therapies for NHL and CLL (B cell-derived lymphomas) vary for each tumor subtype. Chemotherapy remains an important treatment for some lymphomas, in some cases, surgery or radiation therapy may be considered. The antibody rituximab directed against CD20, a surface protein present on B cells in most development phases, has shown significant activity in clinical studies. By binding to CD20 on lymphoma cells, the antibody may induce cell death by complement-dependent cytotoxicity (CDC) or by antibody-dependent cell-mediated cytotoxicity (ADCC). Rituximab has been used to treat various NHL diseases in different disease stages (Mohammed et al., 2019). For aggressive diseases, rituximab may be combined with a chemotherapy treatment regime such as CHOP (cyclophosphamide, doxorubicin, vincristine and prednisone).

The therapies mentioned above are surprisingly improved by adding and applying anapoptotically active substance, preferably a drug, more preferably a pharmaceutical composition of the present invention.

A preferred subject of the instant invention is a method of disorders as described herein, wherein the disorders are selected from cancer and/or metastatic cancer. Preferably, the metastatic cancer is selected from the group consisting of metastatic breast cancer, metastatic lung cancer, metastatic pancreatic cancer, metastatic head and neck cancer, metastatic prostate cancer, metastatic colorectal cancer, metastatic liver cancer, metastatic pancreatic cancer, metastasis in liver and lung, and metastatic malignant melanoma.

In the context of the instant invention the metastases are preferably qualified or named by the organ they metastasized to.

According to the instant invention, the metastases are preferably selected from the group consisting of bone metastases, lung metastases, liver metastases and brain metastases, more preferably selected from group consisting of bone metastases, lung metastases and brain metastases and especially preferably selected from group consisting of bone metastases and brain metastases.

According to the invention, the metastases preferably include lymph node metastases, even more preferably distant lymph node metastases. Thus, a preferred subject of the instant invention relates to a method of treating disorders as described above and/or below, wherein the disorder to be treated are lymph node metastases.

According to the instant invention, the cancer is preferably selected from the group consisting of breast cancer, lung cancer, head and neck cancer, prostate cancer, brain cancer, colorectal cancer, liver cancer, pancreatic cancer, malignant melanoma lymphoma, including Hodgkin lymphoma and non-Hodgkin lymphoma, and Chronic Lymphocytic Leukaemia (CLL), more preferably selected from the group consisting of breast cancer, pancreatic cancer, melanoma, lymphoma, including Hodgkin lymphoma and non-Hodgkin lymphoma, and CLL, even more preferably selected from the group consisting of breast cancer, pancreatic cancer, malignant melanoma and CLL, especially preferably is selected from the group consisting of breast cancer and pancreatic cancer. Alternatively preferably, the cancer is selected from the group consisting of malignant melanoma, lymphoma, including Hodgkin lymphoma and non-Hodgkin lymphoma, and CLL.

The term “breast cancer” as used in the context of the present invention preferably includes:

hormone receptor negative breast cancer,
hormone receptor positive breast cancer,
HER2 negative breast cancer,
HER2 positive breast cancer,
hormone receptor negative, HER2 negative breast cancer,
hormone receptor positive, HER2 negative breast cancer,
hormone receptor negative, HER2 positive breast cancer, and/or
hormone receptor positive, HER2 positive breast cancer.

The term “breast cancer” as used in the context of the present invention preferably includes “normal” breast cancer” or “non-metastatic breast cancer”, and/or “metastatic breast cancer”.

The term “non-metastatic breast cancer” preferably includes:

non-metastatic hormone receptor negative breast cancer,
non-metastatic hormone receptor positive breast cancer,
non-metastatic HER2 negative breast cancer,
non-metastatic HER2 positive breast cancer,
non-metastatic hormone receptor negative, HER2 negative breast cancer,
non-metastatic hormone receptor positive, HER2 negative breast cancer,
non-metastatic hormone receptor negative, HER2 positive breast cancer, and/or
nonmetastatic hormone receptor positive, HER2 positive breast cancer.

The term “metastatic breast cancer” is preferably selected from:

metastatic hormone receptor negative breast cancer,
metastatic hormone receptor positive breast cancer,
metastatic HER2 negative breast cancer,
metastatic HER2 positive breast cancer,
metastatic hormone receptor negative, HER2 negative breast cancer,
metastatic hormone receptor positive, HER2 negative breast cancer,
metastatic hormone receptor negative, HER2 positive breast cancer, and/or
metastatic hormone receptor positive, HER2 positive breast cancer.

Those terms are known and understood in the art.

Thus, a preferred subject of the instant invention relates to a method of treatment as described herein, wherein the disorders to be treated are one or more disorders, selected from the groups consisting of

i) bone, brain, lung and/or liver metastases of breast cancer,
ii) brain, bone, lung and/or liver metastases of lung cancer,
iii) brain metastases of malignant melanoma,
iv) bone and/or liver metastases of colorectal cancer,
v) bone metastases of prostate cancer,
vi) lung, liver and/or bone metastases of head and neck cancer, and
vii) liver, lung and intestine metastases of pancreatic cancer.

More preferably, the disorders to be treated are one or more disorders, selected from the group consisting of brain metastases of lung cancer, brain metastases of malignant melanoma, brain metastases of breast cancer, bone metastases of breast cancer, bone metastases of prostate cancer, bone metastases of colorectal cancer, liver metastases of colorectal cancer and liver metastases of pancreatic cancer. Even more preferably, the disorders to be treated are one or more disorders selected from the group consisting of bone metastases of breast cancer, bone metastases of colorectal cancer and/or bone metastasis of prostate cancer. Alternatively preferably, the disorders to be treated are selected from the group consisting of brain metastases, preferably brain metastases of breast cancer, brain metastases of lung cancer and/or brain metastases of malignant melanoma, and especially preferably brain metastases of lung cancer. Alternatively preferably, the disorders to be treated are selected from the group consisting of lung metastases of pancreatic cancer, lung metastases of breast cancer and/or lung metastases of malignant melanoma.

Thus, a preferred subject of the instant invention relates to a method of treatment as described herein, wherein the lung cancer is selected from non-small cell carcinoma (NSCLC) and small cell carcinoma (SCLC), the head and neck cancer is squamous cell carcinoma of the head and neck (SCCHN), the liver cancer is hepatocellular carcinoma (HCC) and/or the brain cancer is selected from astrocytoma, glioblastoma and glioblastoma multiforme.

Thus, a preferred subject of the instant invention relates to a method of treatment as described herein, wherein the disorder to be treated is selected from non-small cell carcinoma (NSCLC) and/or metastases thereof, and especially preferably selected from non-small cell carcinoma (NSCLC) and/or brain metastases thereof.

Thus, a preferred subject of the instant invention relates to a method of treatment as described herein, wherein the disorder to be treated is selected from brain metastases of non-small cell carcinoma (NSCLC).

Thus, a preferred subject of the instant invention relates to a method of treatment as described herein, wherein the disorder to be treated is selected from small cell carcinoma

(SCLC) and/or metastases thereof, and especially preferably selected from small cell carcinoma (SCLC) and/or brain metastases thereof.

Thus, a preferred subject of the instant invention relates to a method of treatment as described herein, wherein the disorder to be treated is selected from brain metastases of small cell carcinoma (SCLC).

Thus, a preferred subject of the instant invention relates to a method of treatment as described herein, wherein the disorder to be treated is selected from squamous cell carcinoma of the head and neck (SCCHN) and/or metastases thereof, and especially preferably selected from squamous cell carcinoma of the head and neck (SCCHN) and/or metastases thereof in the lung, liver and/or bone.

Thus, a preferred subject of the instant invention relates to a method of treatment as described herein, wherein the disorder to be treated is selected from lung, liver and/or bone metastases of squamous cell carcinoma of the head and neck (SCCHN).

Thus, a preferred subject of the instant invention relates to a method of treatment as described herein, wherein the disorder to be treated is selected from hepatocellular carcinoma (HCC) and/or metastases thereof, and especially preferably selected from hepatocellular carcinoma (HCC).

Thus, a preferred subject of the instant invention relates to a method of treatment as described herein, wherein the disorder to be treated is selected from colorectal cancer and/or metastases thereof, and especially preferably selected from colorectal cancer and/or liver or bone metastases thereof.

Thus, a preferred subject of the instant invention relates to a method of treatment as described herein, wherein the disorder to be treated is selected from liver metastases of colorectal cancer.

Thus, a preferred subject of the instant invention relates to a method of treatment as described herein, wherein the disorder to be treated is selected from malignant melanoma and/or metastases thereof, and especially preferably selected from malignant melanoma and/or brain metastases thereof.

Thus, a preferred subject of the instant invention relates to a method of treatment as described herein, wherein the disorder to be treated is selected from brain metastases of malignant melanoma.

Thus, a preferred subject of the instant invention relates to a method of treatment as described herein, wherein the disorder to be treated is selected from breast cancer and/or metastases thereof, and especially preferably selected from breast cancer and/or bone or brain metastases thereof.

Thus, a preferred subject of the instant invention relates to a method of treatment as described herein and, wherein the disorder to be treated is selected from bone metastases of breast cancer.

Thus, a preferred subject of the instant invention relates to a method of treatment as described herein, wherein the disorder to be treated is selected from prostate cancer and/or metastases thereof, and especially preferably selected from prostate cancer and/or bone metastases thereof.

Thus, a preferred subject of the instant invention relates to a method of treatment as described herein, wherein the disorder to be treated is selected from bone metastases of prostate cancer.

Thus, a preferred subject of the instant invention relates to a method of treatment as described herein, wherein the disorder to be treated is selected from pancreatic cancer and/or metastases thereof, and especially preferably selected from pancreatic cancer and/or liver or lung metastases thereof.

Thus, a preferred subject of the instant invention relates to a method of treatment as described herein and, wherein the disorder to be treated is selected from liver metastases of pancreatic cancer.

Preferably, the method of treating disorders as described herein, can be advantageously combined with radiotherapy. Even more preferably, the method of treating disorders, selected from cancer and/or metastases thereof as described herein, can be advantageously combined with radiotherapy. Said methods can be even more preferably combined with concurrently or consecutively administered radiotherapy. Radiotherapy in this regard is preferably selected from radioimmunotherapy and external beam radiation, and more preferably is external beam radiation.

Thus, a preferred subject of the instant invention relates to a method of treating a subject as described herein, wherein said subject also receives or received, preferably receives radiotherapy, preferably radiotherapy as described herein.

Thus, even more preferred is a method of treating a subject as described herein, wherein said methods (additionally) comprises administering radiotherapy to said subject. Especially preferred is a method of treating a subject as described herein, wherein said methods (additionally) comprises administering radiotherapy concurrently or consecutively to said subject. Radiotherapy in this regard is preferably external beam radiation.

According to the instant invention, radiotherapy is preferably external beam radiation. The terms “radiotherapy” and “external beam radiation” in this regard are known and understood in the art. Preferably, external beam radiation includes, but is not limited to, single dose external beam radiation or single dose radiation, fractionated external beam radiation or fractionated radiation, focal radiation and whole organ irradiation, such as whole brain radiation. Radiation in this regard is preferably also referred to as irradiation.

Typically, the external beam radiation is photon radiation and/or gamma radiation.

The amount of radiation used in external beam radiation and/or photon radiation therapy is measured in gray (Gy), and varies depending on the type and stage of cancer being treated. For curative cases, the typical dose for a solid tumor ranges from 60 to 80 Gy. Preventative and/or adjuvant doses are typically around 45-60 Gy in 1.8-2 Gy fractions (e.g. for Breast, Head, and Neck cancers.) Suitable doses and dosing schedules are known to the skilled artisan.

Preferably the present invention provides a method, wherein the treatment of the bone metastases comprises or induces

a) reduced bone resorption, preferably reduced osteoclast-mediated bone resorption,
b) new bone formation, preferably new bone formation in the osteolytic lesions,
c) regulation or normalisation of the osteoclast activity,
d) resumption of bone formation,
e) regrowth of bone or partial regrowth of the bone,
in said subject.

The term “at least one” preferably comprises the terms “at least two” and/or “at least three”, and preferably the like. The term “at least one” thus preferably includes “one”, “two”, “three” and preferably also higher numbers.

The term “one or more” preferably has the same meaning as “at least one”, and thus preferably also includes the meanings “two or more” and/or “three or more”, and preferably the like. The term “one or more” thus preferably also includes “one”, “two”, “three” and preferably also higher numbers.

If not explicitly stated otherwise, the term “solid composition” or “solid compositions” preferably exclusively refers to such compositions that are free of water or essentially free of water. Essentially free of water with regard to said solid compositions means a residual water content of less than 10%, more preferably less than 5%, even more preferably less than 2% and especially preferably less than 1%, e.g. 0.001 to 5% or 0.01 to 2%, preferably based on the total weight of the (dried) composition.

If not explicitly stated otherwise, the term “composition” or “compositions” in the absence of the term “solid” preferably refers to both
a) “non-solid compositions”, i.e. compositions that preferably have a water content of more than 1%, more preferably a water content of more than 2%, even more preferably a water content of more than 5% and especially a water content of more than 10%, preferably based on the total weight of the respective composition, and
b) “solid compositions”, preferably as defined above.

However, if not explicitly stated otherwise, the amounts given herein for the respective ingredients in the compositions in the absence of the term “solid” preferably refer to the amounts in “non-solid compositions”, preferably water-based compositions as described herein, and even more preferably refer to suspensions and especially preferably aqueous suspensions as described herein and/or solution and especially preferably aqueous solution.

Preferably, the compositions according to the invention do not contain one or more antigens. More preferably, the compositions according to the invention are free or essentially free of antigens or compounds that act as antigens.

Preferably, the oligopeptide(s) contained in the compositions according to the invention do not act as an antigen.

Preferably, the compositions according to the invention do not contain one or more anticonvulsant agent. More preferably, the compositions according to the invention are free or essentially free of antigens or compounds that act as an anticonvulsant agent.

Preferably, the oligopeptide(s) contained in the compositions according to the invention do not act as an anticonvulsant agent.

Preferably, the compositions according to the invention do not contain one or more anti-retroviral agents. More preferably, the compositions according to the invention are free or essentially free of anti-retroviral agents or compounds that act as an anti-retroviral agent.

Thus, the compositions according to the invention preferably contain only minor amounts of or are especially preferably free or essentially free of natural amphiphilic compounds and/or naturally derived amphiphilic compounds. Such natural amphiphilic compounds or naturally derived amphiphilic compounds include, but are preferably not limited to natural cholines, such as egg phosphatidylcholine, soy phosphatidylcholine, lecithin and the like. Minor amounts in this regard are preferably 0.5% or less, 0.1% or less, 0.01% or less, 0.001% or less, or 0.0001% or less, based on the amount of the one or more oligopeptides or cyclic oligopeptides as described herein contained in said composition. Percentages in this regard are preferably mole-% or % w/w, more preferably % w/w.

The term “ad. 100%”, “add 100%” and/or “add. 100%” with respect to a component of a composition is known in the art. Preferably, it means that this component is added to the other given components until 100% of the composition or total composition is achieved. Accordingly, the term “ad. 100 v %” preferably means that this component is added to the other given components until 100 v % of the composition or total composition is achieved, and the like.

A preferred subject of the instant invention is a method or a use as described herein, wherein the medicament is to be used in the treatment of recurrent cancer, for example in a second line or subsequent treatment setting.

A more preferred subject of the instant invention is a method or a use as described herein, wherein the medicament is to be used in the treatment of recurrent cancer, for example in a second line or subsequent treatment setting, wherein the cancer is as defined herein.

A method or a use according to one of the preceding claims, wherein the medicament is to be used in the treatment of newly diagnosed cancer, preferably in a first line chemotherapy setting.

Another preferred subject of the instant invention is a method or a use as described herein, wherein the medicament is to be used in the treatment of newly diagnosed cancer, preferably in a first line treatment setting, wherein the cancer is selected from the group consisting of astrocytoma, more preferably astrocytoma grade II, III and/or IV, and especially consisting of glioblastoma or glioblastoma multiforme.

A further subject of the instant invention is a method of treatment of a subject, preferably a human subject, or a use as described herein and/or the pharmaceutically acceptable salts thereof, wherein the treatment or use concerns newly diagnosed cancer, preferably in a first line chemotherapy setting.

A further subject matter of the present invention is a set (kit) consisting of separate packs of

(a) an effective amount of a apoptotically active substance according to the present invention and/or pharmaceutically acceptable salts, tautomers and stereoisomers thereof, including mixtures thereof in all ratios, and
(b) an effective amount of a further medicament active ingredient.

A further subject matter of the present invention is an apoptotically active substance according to the present invention and/or pharmaceutically acceptable salts thereof being labelled.

Under a further preferred aspect of the present invention, these peptides or correspondingly derivatized peptides can also be used as diagnostic agents. As was shown by the inventors, the substances according to the invention, and preferably the peptides of the present invention, bind to apoptotic cells and are therefore ideally suited for use as a diagnostic tool, for example so as to detect apoptotic cells in the area of atherosclerotic lesions, and thus the lesions, in the first place. The substances are labelled for this purpose. A plurality of methods are known for this labelling to a person skilled in the art, which are selected depending on the application purpose.

Suitable methods are described, for example, in the US patent specifications U.S. Pat. No. 4,479,930 (Hnatowich), U.S. Pat. No. 4,652,440 (Paik et al) and U.S. Pat. No. 4,668,503 (Hnatowich).

For the purposes of the present invention, a “labelled substance according to the invention” is thus understood to mean any substance according to the invention which includes labelling substances known from the prior art and used as a standard for labelling peptides, such as in general radio isotopes such as rhenium or technetium, but also enzymes, enzyme substrates, antibodies, epitopes for identification via specific antibodies/fragments and the like. A person skilled in the art will readily identify the above enumeration as exemplary and not exhaustive.

If the substances according to the invention are to be used as diagnostic tools, any labelling method known in the prior art can be used.

Preferably, a reference to a pharmaceutically active substance of the present invention includes also the pharmaceutically acceptable derivatives, solvates and/or salts thereof.

Preferably, a reference to a pharmaceutically active substance of the present invention preferably includes the pharmaceutically acceptable derivatives, solvates and/or salts thereof.

Thus, a reference to a pharmaceutically active substance of the present invention acceptable derivatives, solvates and/or salts thereof preferably refers to a pharmaceutically active substance of the present invention and/or the pharmaceutically acceptable derivatives, solvates and/or salts thereof.

If not explicitly defined otherwise, the naming of an active ingredient, active principle (API), medicament or international nonproprietary name (INN) thereof preferably includes all prodrugs, salts and solvates thereof, especially those that are functionally equivalent and/or are deemed a suitable substitute from a clinical point of view.

If not explicitly defined otherwise, the terms humans, human beings, human patients or patients are preferably used herein as interchangeable or as synonyms.

The term “without a pause” as used herein, especially used with respect to treatment regimens or treatment durations, is preferably understood to mean that said treatment regimens or durations are performed or applied in a consecutive order. For example, “2 to 8 weeks and especially 6 weeks, preferably without a pause” is preferably intended to mean “2 to 8 weeks and especially 6 weeks, preferably in a consecutive order”.

If not specified otherwise, amounts administered to a patient given in “mg”, such as in 500 mg, 1000 mg, 2000 mg, etc., are preferably intended to mean the respective amounts to be administered “flat”, i.e. as a fixed dose that is not adjusted to the bodyweight and/or body surface of the respective patient.

Especially preferred according to the invention are subjects as described herein, wherein the characteristics of two or more preferred, more preferred and/or especially preferred embodiments, aspects and/or subjects are combined into one embodiment, aspect and/or subject. Preferably, according to this invention, preferred subjects or embodiments can be combined with other preferred subjects or embodiments; more preferred subjects or embodiments can be combined with other less preferred or even more preferred subjects or embodiments; especially preferred subjects or embodiments can be combined with other just preferred or just even more preferred subjects or embodiments, and the like.

The term “about” as used herein with respect to numbers, figures, dosings, hours, ranges and/or amounts is preferably meant to mean “circa” and/or “approximately”. The meaning of those terms is well known in the art and preferably includes a variance, deviation and/or variability of the respective number, figure, dosings, hours, range and/or amount of plus/minus 15%, especially of plus/minus 10% and preferably of plus/minus 5%.

The terms “disorder(s)” and “disease(s)” as used herein are well-known and understood in the art. In the context of the present invention they are preferably used as synonyms and thus are preferably interchangeable, if the context they are used herein does not strongly implicate otherwise.

In the medical context, including, but not limited to treatment regimens, dosing schedules and clinical trial designs, for convenience and/or ease of use by patients, medical staff and/or physicians, as well as reliability and/or reproducibility of results etc., the terms “week”/“a week”, “month”/“a month” and/or “year”/“a year” can be used with slight deviations from the definitions of the gregorian calendar. For example, in said medical context, a month is often referred to as 28 days, and a year is often referred to 48 weeks.

Thus, in the context of the instant invention, the term “week” or “a week” preferably refers to a period of time of about 5, about 6 or about 7 days, more preferably about 7 days.

In the medical context, the term “month” or “a month” preferably refers to a period of time of about 28, about 29, about 30 or about 31 days, more preferably about 28, about 30 or about 31 days.

In the medical context, the term “year” or “a year” preferably refers to a period of time of about 12 months or to a period of time of about 48, about 50, or about 52 weeks, more preferably 12 months, or about 48 or about 52 weeks.

A method or a use according to one of the preceding claims, wherein the medicament is to be used in the treatment of newly diagnosed cancer, preferably in a first line chemotherapy setting.

Moreover, the following examples are given in order to assist the skilled artisan to better understand the present invention by way of exemplification. The examples are not intended to limit the scope of protection conferred by the claims. The features, properties and advantages exemplified for the compounds, compositions, methods and/or uses defined in the examples may be assigned to other compounds, compositions, methods and/or uses not specifically described and/or defined in the examples, but falling under the scope of what is defined in the claims.

Preferably, the features, properties and advantages exemplified for the compounds, compositions, methods and/or uses defined in the examples and/or claims may be assigned to other compounds, compositions, methods and/or uses not specifically described and/or defined in the examples and/or claims, but falling under the scope of what is defined in the specification and/or the claims.

The invention is explained in greater detail below by means of examples. The invention can preferably be carried out throughout the range claimed and is not restricted to the examples given here.

Thus, the following examples describe the invention in more detail but do not limit the invention and its scope as claimed.

### EXAMPLES

The following examples are given in order to assist the skilled artisan to better understand the present invention by way of exemplification. The examples are not intended to limit the scope of protection conferred by the claims. The features, properties and advantages exemplified for the compounds and uses defined in the examples and/or the Figures related thereto may be assigned to other compounds and uses not specifically described and/or defined in the examples and/or the Figures related thereto, but falling under the scope of what is defined in the claims.

### Materials Used:

### Cell Lines and Primary Cells:

A375, human melanoma, obtainable from the American Type Culture Collection (ATCC).

BxPC-3, human pancreatic cancer cell line, obtainable from DSMZ Braunschweig, Germany.

MEC-1, human B-chronic lymphocytic leukemia cell line, obtainable from DSMZ Braunschweig, Germany.

Lymphocytes, human peripheral blood mononuclear cells (“PBMC”) containing mostly lymphocytes, were obtained from a donor.

MDA-MB-231, Breast Carcinoma cell line, obtainable from DSMZ Braunschweig, Germany.

### Materials and Solutions

50 m1 tube: Carl Roth, Karlsruhe

25 cm² and 75 cm² cell culture bottles: Nunc, Sigma-Aldrich, Darmstadt

6-well and 48-well cell culture plates, Sarstedt, NUmbrecht

A375 culture medium: DMEM medium (Biochrom, Berlin) with 10% (v/v) FCS

Biotase: Biochrom, Berlin

Biocoll: Biochrom, Berlin

BxPC-3 culture medium: RPMI 1640 with 10% (v/v) FCS

DAPI: (4′, 6-diamidino-2-phenylindole), Biolegend, San Diego, USA

DAPI solution: 2 mM MgCl2, 155 mM NaCl, 0.1% (v/v) Triton X-100, 60 nM DAPI, 0.1 M Tris-HCl pH 7.3

DMEM: Dulbecco's MEM, Biochrom, Berlin

DMSO: 99.8% p.a., Carl Roth, Karlsruhe

Ethanol p. a.: Carl Roth, Karlsruhe

Etoposide: from stock solution (20 mg/ml, Hexal, Holzkirchen) is diluted with culture medium used in a final concentration of 86 μ/ml, in parallel to the peptide solutions.

FACS tubes and ventilation plugs, Sarstedt, Nümbrecht

FCS: fetal calf serum, Biochrom, Berlin

Heparin tubes: Sarstedt, Nümbrecht

Iscove's medium: Biochrom, Berlin

LeucoSep tubes: Greiner bio-one, Frickenhausen

MEC-1 culture medium: Iscove's medium (IMDM) with 10% FCS

Lymphocyte culture medium: Iscove's medium (IMDM) with 10% FCS

MDA-MB-231 culture medium: DMEM medium (Biochrom, Berlin) with 10% (v/v) FCS.

PBS: Dulbecco's Phosphate buffered saline (PBS Dulbecco), Biochrom,

Berlin

Peptides: Peptide stocks were set at 20 mM in 50% DMSO (v/v); as control solution (without peptide) 50% DMSO (v/v) is used.

PI buffer: 0.1% (v/v) Triton X-100; 10 μg/ml propidium iodide; 100 μg/ml RNase A (DNase/protease-free) in PBS buffer

Propidium iodide, Sigma, Deisenhofen

Reaction vessels 2.0 ml, Sarstedt, Nümbrecht

RNase A (DNase-free), ThermoFisher, Darmstadt

RPMI 1640 (Roswell Park Memorial Institute 1640) medium: Biochrom, Berlin

Trypsin/EDTA solution: 0.05% trypsin, 0.02% EDTA in PBS

Versene solution: 1% EDTA-sodium salt in PBS

Centrifuge tube: 15 ml Cellstar, Greiner Bio-One

### Cell Cultivation

Adherent cells are cultured in the respective medium in cell culture flasks or cell culture plates. For passages, the cells are washed with PBS, incubated with biotase or trypsin for 5 to 10 min at 37 degrees Celsius, centrifuged at a maximum of 130 g, counted and reseeded in a certain cell count per area or per ml of medium.

Suspension cells are also cultured in cell culture flasks or cell culture plates. For the reaction, they are diluted without enzyme treatment in fresh culture medium.

### Isolation and Handling of Cell Suspensions for FACS Analysis

MEC-1 cells are cultured in MEC-1 culture medium at 37 degrees Celsius and 5% CO₂. For induction with peptides, 0.8×10⁶ to 1×10⁶ cells are seeded in each well of 48-well plates.

Human lymphocytes are obtained as peripheral blood mononuclear cells (“PBMC”) from whole blood. For this purpose, 15 ml of heparinized whole blood (heparin tube, Sarstedt) were layered on 15 ml Biocoll in LeucoSep tubes and obtained by centrifugation (10 min at 120 g) as the top layer (“lymphocytes”) and so separated from other cells. For induction with peptides, 1×106 lymphocytes are seeded into each well of the 48-well plates.

After addition of the peptides diluted in culture medium to the cells (MEC-1 or lymphocytes) in the respective cavity, the cells are cultured for 2 to 4 hours at 37 degrees Celsius and 5% CO2. The cell suspension is centrifuged in centrifuge tubes at 120 g, the cells are suspended in 500 μl serum-free culture medium, then 1.5 ml of cold 80% ethanol (ethanol/PBS-80/20, v/v; stored at −20 degrees Celsius) is added, the FACS tubes sealed and stored at −20 degrees Celsius for a maximum of 18 h. The cells are centrifuged for 5 min at 120 g and suspended in 2 ml PBS, then incubated for 30 min at 37 degrees Celsius. After renewed centrifugation, the cells are suspended in 0.5 ml DAPI solution and incubated for 15 min at 20 degrees Celsius in the dark. Thereafter, the cells are stored dark for a maximum of four hours in an ice bath, then the FACS analysis.

### Implementation of the Cell Cultivation and Harvesting of Adherent Cells for FACS Analysis

For each cell line, the indicated culture medium is used in each case (A375-or BxPC-3 culture medium; or MEC-1-or lymphocyte culture medium, or MDA-MB-231 culture medium) the following it is generally referred to as culture medium. The cells are cultured at 37 degrees Celsius and 5% CO2. Alternatively, two protocols are used for the preparation and execution of the FACS analyzes.

### Protocol A:

To transfer adherent cells (A375 and BxPC-3 cells) after culture, cells are washed twice with PBS. To detach the cells, the enzyme biotase is used according to the manufacturer's instructions. For this purpose, after washing with PBS, the cells are mixed with biotase (5 ml of biotase solution per 75 cm² surface of the cell culture bottles) and incubated for 5 min at 37 degrees Celsius, and the biotase is separated by centrifugation. Cells cultivated in suspension (MEC-1 and lymphocytes) were transferred by dilution. The cells are suspended in culture medium and counted, each time 1.5×10⁵ cells/cm² are seeded in 48-well plates and cultured for between 1.5 and 24 hours at 37 degrees Celsius, 5% CO2. Peptides are pre-diluted from stock solution with culture medium and 200 μl of peptide solution added so that final concentrations of 30 to 500 μM peptide are present in at most 1% (v/v) DMSO. As control solutions, culture medium without peptide (DMSO-containing) in culture medium or etoposide solution is added. After incubating the cells for several hours, the cell supernatant is transferred to a FACS tube for each batch and, after washing the cells with PBS, the wash solution is added. Then the cells are incubated with 200 μl of biotase solution for 10 min, 37° C., the cells with the biotase are also transferred to the FACS tube and the cells are collected by centrifugation for 5 min. The cell pellet is suspended in 500 μl of serum-free culture medium, then 1.5 ml of cold 80% ethanol (ethanol/PBS-80/20, v/v, stored at −20° C.) is added, the FACS tubes sealed and stored at −20 degrees Celsius for 15 to 18 h. Thereafter, the cells are centrifuged for 5 minutes at 120 g, suspended after removal of the supernatant in 2 ml PBS and incubated at 37 degrees Celsius for 30 minutes. After re-centrifugation, the cells are suspended in 0.5 ml DAPI solution and incubated for 15 min at 20 degrees Celsius in the dark. Thereafter, the cells are stored dark for a maximum of four hours in an ice bath, followed by the FACS analysis.

### Protocol B:

The adherent cells (A375 and BxPC-3 cells and MDA-MB-231, respectively) are seeded 24 hours before induction in 6-well plates in culture medium. For this, the cells are washed with PBS, then incubated with trypsin/EDTA solution at 37 degrees Celsius and suspended in culture medium. The 6-well plates are loaded with 1×10⁵ cells/cm² in culture medium and the cells incubated for 24 hours until induction with the peptides in the incubator. For induction, the peptides are diluted in fresh culture medium. As control solutions, culture medium without peptide (DMSO-containing) in culture medium or etoposide solution is added. The cells are washed with PBS. Thereafter the peptide solution in culture medium is added to the cells. After incubation in the incubator (37° C., 5% CO₂) between 1.5 and 24 hours, the cell supernatant is transferred to 15 ml centrifuge tubes, the cells are washed with 0.75 ml PBS and then with 0.5 ml versene solution. Both washings are combined with the cell supernatant. The cells are then added with 0.5 ml of trypsin/EDTA solution and incubated at about 37° C. for at most 10 minutes until the cells detached. The cell suspension is combined with cell supernatant and washings and centrifuged at about 130 g for about 10 minutes. After removing the supernatant, the cells are suspended in 400 μl of cell culture medium (without serum) and mixed with 1.2 ml of 80% ethanol/20% PBS (v/v) and incubated for 1 to 7 days at −20° C. For analysis, the cells are centrifuged at 130 g for 10 min, suspended in 2 ml PBS, and centrifuged again for 5 min. The cells are suspended in 700 μl PI buffer and incubated for 10 min at 37° C. in the dark. Subsequently, the cells are stored dark for a maximum of four hours in an ice bath, then the FACS analysis is performed.

### Evaluation of the Results of the FACS Analysis

The proportion of cells which have died was determined by two methods, both rely on the analysis of cells by flow cytometry, often called FACS analysis. This technology allows to determine the physical and chemical properties of both normal cells and tumor cells. The cells can be labelled by fluorescent dyes, which may bind to cellular DNA or which are coupled to antibodies that bind to proteins or other structures on the cell surface or inside the cells. The cells are suspended and injected into a flow cytometer instrument and, ideally one at a time, the cells flow through a laser beam. The laser light is either scattered or elicits fluorescence at the dyes which were applied and the scattered and the fluorescent light is analyzed by an optical and light detection system. This system with an array of dichroic mirrors or prisms and optical filters serves to direct the light to photomultipliers that are mounted in defined positions, either in line of the laser and the position where the cell meets the laser beam, thereby delivering signals of the forward-scattered light (FSC); or photomultipliers detect light which is scattered in an angle relative to the FSC beam direction, to detect the side-scattered light (SSC), or to detect selected wave length from fluorescent light. Programmed cell death (apoptosis) can be detected by changes of the FSC/SSC light scattering pattern, or by the appearance of cells with a low DNA content, as revealed by the so called subG1 peak (Darzynkiewicz et al., 1992; Vermes et al., 2000; see below). Further information and useful handling instructions are available in Shapiro, H. M. “Practical Flow Cytometry”, 4th Edition, Beckman Coulter Life Sciences, ISBN: 978-O-471-41125-3, (2003), eBook, https://www.beckman.com/resources/reading-material/ebooks/practical-flow-cytometry/

### Flow Cytometers:

For suspension cells (MEC-1 and lymphocytes/PBMC) and for adherent cells analyzed according to Protocol A, FACS analysis to determine subG1 cell cycle and vital cell fractions was done on a Becton Dickinson LSR Fortessa flow cytometer. The analysis by FACS including subG1 and vital cell analysis according to protocol B was performed with a Cytomics FC 500 MPL flow cytometer from Beckman Coulter.

### Vital Cells Analysis:

Changes of the cell form during cell death by apoptosis are documented in the literature (Lizard et al. 1995; Taga et al. 2000; Vermes et al., 2000). The protocols for cell cultivation, treatment and preparation of the cells before flow cytometry is detailed in separate examples. Cell suspensions of cell lines A375, BxPC-3, MEC-1 or of primary lymphocytes (see separate example of lymphocyte preparation) are analyzed by flow cytometry. The data are further analyzed by using the program “Flowing Software 2”. The data of light scattering are drawn in a two-dimensional FSC/SSC plot. Since the position in the FSC/SSC plot of apoptotic cells changes relative to non-apoptotic cells (Vermes et al., 2000), a region (R-1) is drawn representing mainly non-apoptotic cells and excluding cells with both low FSC and low SSC values and excluding cells with high values of FSC and/or SSC (FIG. 1). The regions are individually selected for each cell type (A375, BxPC-3, MEC-1 and primary lymphocytes). The percentage of untreated non-apoptotic cells in the region R-1 relative to total cells is set to 100%, representing 100% vital cells. The fraction (%) of cells in R-1 relative to the total number of cells in case of cell treatment by certain peptides or by cytotoxic drugs results in cell vitality, below 100%, thereby indicating a loss of cell vitality by the treatment.

The analysis of the normal cell form after appropriate treatment with a peptide is carried out by determining the percentage of cells in the region R-1 in the diagram (application Forward Scatter versus Sideward Scatter). The reference is the percentage of cells in R-1 without peptide addition (solvent DMSO only). Here, the proportion of cells without peptide addition in region R-1 was set as “100% normal cell shape”, which corresponds to 100% vitality. At values less than 100% in R-1, the proportion of vital cells decreases accordingly.

Cell Cycle Analysis (subG1 Values)

The second method analyzes the cell cycle phases which is drawn from the relative DNA content. Cells labeled by a fluorescent dye, using either DAPI or propidium iodide (PI) were analyzed by a cell sorter. The protocols for cell cultivation, treatment and preparation and staining of the cells for the flow cytometry is given in separate examples. Following flow cytometry, the frequency of fluorescence intensities which is proportional to the DNA content is plotted in a single dimension histogram, which reveals whether the cells are either in the Go/G1 phase (resting cells); or the cells proliferate and are in the DNA synthesis (S) phase or in the G2/M phase which is before or at the time of cell division (mitosis). When cells die by apoptosis, cellular changes lead to diminished DNA content resulting in a new peak located before the G₀/G1 peak, often called “subG1” peak (Broecker-Preuss et al. 2015; Darzynkiewicz et al., 1992; Dbaibo et al. 1998; Fischbeck et al. 2011).

The “Flowing Software 2” program is used to draw from the raw data of the flow cytometer an FSC/SSC plot. This first step is performed in order to analyze a selected cell fraction excluding cells with both small FSC and small SSC values. Therefore, a gating area is drawn in a two-dimensional FSC/SSC plot (region R-1) which contains mainly intact cells, but no small vesicles. This is initially done for each cell type using reference cells which were not incubated with substances inducing cell death. The geometry applied for region R-1 is usually a pentagon, as shown in FIG. 1. By this selection of the cell fraction, a bias is prevented which would include a very high number of small cellular vesicles (apoptotic bodies) that are formed by membrane blebbing from dying cells, which have a low DNA content but which, by their large number, would increase the fraction of presumptive subG1 cells. Next, a one dimensional plot analyzing only this gated cell fraction is drawn which shows the distribution of the DNA content and, in case of the untreated control cells, gives a typical cell cycle profile with G₀/G1-, S- and G2/M phases. In this DNA content plot, an analytical region is drawn in the area of lower DNA content to count the cells that show a subG1 peak, as illustrated in FIG. 1. The fraction of apoptotic cells is defined as the fraction of cells with DNA content in the subG1 region as part of the total number of cells in the DNA content plot (FIG. 1).

In the plot of side-scattered light (SSC) versus forward-scattered light (FSC), the full scale linear plot for each, SSC and FSC, was normalized to 100%. As shown in FIG. 1, 100% corresponds to an intensity value of 262114 units.

The coordinates for the corner points (m1 to m5) of the analysis of adherent cells (A375 and BxPC-3) according to protocol A (data from the Becton Dickinson flow cytometer) were as follows. Percent of full scale for SSC (S) and FSC (F), respectively: m1 (S=23.1%, F=50.0%); m2 (S=34.6%, F=37.3%); m3 (S=59.6%, F=51.9); m4 (S=57.7%, F=77.7%); m5 (S=38.5%, F=36.9%).

The coordinates for the corner points (ml to m5) of the analysis of MEC-1 cells according to protocol A (data from the Becton Dickinson flow cytometer) were as follows. Percent of full scale for SSC (S) and FSC (F), respectively: ml (S=6.9%, F=18.1%); m2 (S=68.1%, F=30.8%); m3 (S=55.8%, F=52.7%); m4 (S=28.9%, F=44.2%); m5 (S=10.0%, F=28.5%).

The coordinates for the corner points (m1 to m5) of the analysis of lymphocytes (PBMC) according to protocol A (data from the Becton Dickinson flow cytometer) were as follows. Percent of full scale for SSC (S) and FSC (F), respectively: m1, (S=3.1%, F=16.9%)/0); m2 (S=27.3%, F=40.4%); m3 (S=66.5%, F=53.9%); m4 (S=14.2%, F=73.1%); m5 (S=3.5%, F=39.2%).

The coordinates for the corner markers (m1 to m5) of the analysis of A375 and BxPC-3 cells according to protocol B (data from the Beckman Coulter flow cytometer) were as follows. Percent of full scale for SSC (S) and FSC (F), respectively: m1 (S=18%, F=17%); m2 (S=40%, F=37%); m3 (S=35%, F=59%); m4 (S=15%, F=57%); m5 (S=9%, F=36%).

Analysis of FACS measurements is performed using the Flowing Software, version 2.5.1 (Turku Center for Biotechnology, University of Turku, Finland). The files of each FACS analysis are opened as FCS files in the “Flowing Software” program in an “analysis” subprogram. In this case, a two-dimensional application of Forward Scanner (FSC-A) against Sideward Scanner (SSC-A) is used. By comparing the profiles of the respective cell type under normal conditions, i.e. without apoptosis-inducing substances, an area or a region is preferably determined. The determination of the region is well known in the art as mentioned above and below. Preferably a pentagon is determined, which covers a substantial part of the measurement points. The coordinates of the pentagon are given in the previous section (“Vital Cells analysis”). Then this setting is saved as a mask (“analysis”, for example with “region R-1”) for further analysis. This mask (“region R-1”) is used for the whole Example as mentioned above and below.

The evaluation of the FACS analysis to determine the subG1 data was also carried out with the program Flowing Software, version 2.5.1. The fluorescence in the respective analysis channel for DAPI or propidium iodide was plotted as a histogram, the data from the respectively defined region R-1 being utilized (see FIG. 1). In addition to the regions for G1+S and G2/M (H-5 and H6), the region for cells in the apoptotic subG1 phase was determined at lower intensities (in FIG. 1: to the left of H5, here H-4). This histogram was assigned a statistics table, the percentage of cells with subG1 signals was determined as % of Vis in the H-4 region.

### Cultivation and Evaluation of Cells Concerning the Respiration Activity

The cultivation of MDA-MB-231 cells in DMEM medium with 10% (v/v) FCS is done as described above and below in more detail (preferably according to Protocol B). The ATP concentrations are determined following treatment of the cells with peptides for 24 to 48 h, as indicated. “% Respiration-active cells” is the ATP concentration relative to cells without peptide treatment (100%).

ATP concentrations in the cavities where the cells grow was determined by the ATPlite 1 step Luminescence Assay System, an ATP Assay Kit from PerkinElmer. The assay relies on ATP which is released from the cells to start the reaction of the enzyme luciferase from firefly which converts D-luciferin, ATP (adenosine triphosphate) and oxygen (O₂) to oxyluciferin, carbon dioxide (CO₂) and pyrophosphate (diphosphate); and light emitted as luminescence, which is read by a luminometer or other device which can read and quantify the light signal. The resulting luminescence was analyzed on a Berthold TriStar² LB 942 Microplate Reader.

The following peptides are used in the Examples

| Position 1 2 3 4 5 6 7 8 9 10 | SEQ-ID |
|---|---|
| m-v-v-y-f-r | (SEQ C1) |
| w-m-v-v-y-f-r | (SEQID No 1) |
| k-m-v-v-y-f-r | (SEQID No 2) |
| m-v-v-y-f-r-w | (SEQID No 3) |
| m-v-v-y-f-r-k | (SEQID No 4) |
| k-w-m-v-v-y-f-r | (SEQID No 9) |
| w-w-m-v-v-y-f-r | (SEQID No 12) |
| k-k-w-m-v-v-y-f-r | (SEQID NO 25) |
| k-w-m-v-v-y-f-r-k | (SEQID NO 26) |
| k-k-w-m-v-v-y-f-r-k | (SEQID No 27) |
| q-k-w-m-v-v-y-f-r-k | (SEQID No 28) |
| k-s-q-t-v-k-k-w-m-v-v-y-f-r-k | (SEQID NO 29) |
| k-k-w-m-v-v-y-f-r-k-s-s-r | (SEQID NO 30) |
| e-r-s-k-k-w-m-v-v-y-f-r-k | (SEQID NO 31) |
| k-k-w-m-v-v-y-f-r-k-e-a-r | (SEQID NO 32) |
| r-s-t-k-k-w-m-v-v-y-f-r-k | (SEQID NO 33) |
| r-s-t-k-k-w-m-v-v-y-f-r | (SEQID NO 34) |
| r-a-s-k-s-q-t-v-k-k-w-m-v-v-y-f- | (SEQID NO 35) |
| r-k-s-a-r | |
| k-s-q-t-v-q-k-w-m-v-v-y-f-r-k | (SEQID NO 36) |
| s-q-t-v-q-k-w-m-v-v-y-f-r-k | (SEQID NO 37) |
| s-q-t-v-q-k-w-m-v-v-y-f-r | (SEQID No 38) |
| d-k-w-m-v-v-y-f-r-d | (SEQID No 39) |
| r-s-t-q-k-w-m-v-v-y-f-r-k | (SEQID NO 40) |
| k-k-w-m-v-v-y-f-r-k-s-g-s- | (SEQID NO 41) |
| g-s-k-k-w-m-v-v-y-f-r-k | |

The peptides were synthesized by CASLO ApS, Kongens Lyngby, Denmark, or by EMC microcollections GmbH, Tubingen, Germany. Purity was >95%, chloride was the counterion; purity was confirmed by High Pressure Liquid Chromatograpy (HPLC).

### SHORT DESCRIPTION OF THE FIGURES

FIG. 1a on the left side depicts the results of a typical FACS analysis as a 2D plot showing the region R-1 as a pentagon.

FIG. 1b on the right side shows a histogram of the cell cycles including the SubG1 region (H-4) based on the region R-1 as shown in FIG. 1a.

FIG. 1c shows a table of the results as given in FIG. 1b.

FIG. 2 shows the induced cell death caused by apoptosis of A375 melanoma cells as given by the Comparative Example 1 and Examples 1 and 2. The concentration of the peptides is 100 μM and the incubation time is about 24 hours. FIG. 2 presents the percentage portion of cells in the subG1 area of the cell cycle as determined by the FACS method as mentioned above and below. The apoptosis data achieved considers the data as measured for the solvent alone (DMSO). For achieving an evaluation of the significance a fourfold chi square test is performed. The data show a high significance (p<0.01) for all of the peptides evaluated. This high significance is marked by a superscript “a” in the Figure.

FIG. 3 shows the induced cell death caused by apoptosis of A375 melanoma cells as given by the Comparative Examples 2 to 4 and Examples 3 to 11. The concentrations of the peptides are provided in the Figure and are 100 μM, 200 μM and 500 μM, respectively. The incubation time is about 24 hours. FIG. 3 presents the percentage portion of cells in the subG1 area of the cell cycle as determined by the FACS method as mentioned above and below. The apoptosis data achieved considers the data as measured for the solvent alone (DMSO). For achieving an evaluation of the significance a fourfold chi square test is performed. The data show a high significance (p<0.01) for the peptides of the present invention evaluated compared to comparative peptide C1, at all three concentrations. This high significance is marked by a superscript “a” in the Figure.

FIG. 4 shows the induced cell death caused by apoptosis of A375 melanoma cells as given by the Comparative Examples 5 to 7 and Examples 12 to 20. The concentrations of the peptides are provided in the Figure and are 50 μM, 100 μM and 200 μM, respectively. The incubation time is about 24 hours. FIG. 4 presents the percentage portion of cells in the subG1 area of the cell cycle as determined by the FACS method as mentioned above and below. For achieving an evaluation of the significance a fourfold chi square test is performed. The data show a high significance (p<0.01) for all of the peptides evaluated. This high significance is marked by a superscript “a” in the Figure.

FIG. 5 shows the induced cell death caused by apoptosis of A375 melanoma cells as given by the Examples 21 to 30. The concentrations of the peptides are provided in the Figure and are 100 μM and 200 μM, respectively. The incubation time is about 3 hours. FIG. 5 presents the percentage portion of cells in the subG1 area of the cell cycle as determined by the FACS method as mentioned above and below. For achieving an evaluation of the significance a fourfold chi square test is performed. Data show a high significance (p<0.01) for the Peptides SEQID NO 30, SEQID NO 32 and SEQID NO 33 evaluated. This high significance is marked by a superscript “a” in the Figure. The data obtained for Peptides SEQID No 27 and SEQID NO 31 show a lower significance and are marked by a superscript “b” in the Figure.

FIG. 6 shows the induced cell death caused by apoptosis of A375 melanoma cells as given by the Examples 31 to 66. The concentrations of the peptides are provided in the Figure and are 100 μM, 200 μM and 300 μM, respectively. The incubation times are provided in the Figure and are about 1.5 h, 3 h, 6 h and 24 h, respectively. As shown in FIG. 6, the bars are arranged in groups depicting the incubation times, the concentration and the peptides, respectively. From the left to the right side the data provided the Peptides SEQID No 36, SEQID NO 37 and SEQID No 27. Furthermore, the data at a concentration of 300 μM are depicted on the left side for each peptide, while the data at a concentration of 100 μM are depicted on the right side for each peptide. For each concentration and each peptide the incubation times are ordered according to the incubation time wherein the data obtained at an incubation time of 24 h are provided at the left side and the data obtained at an incubation time of 1.5 h are provided at the right side. The data obtained at an incubation time of 6 h are provided at the right side of the 24 h data and the data obtained at an incubation time of 3 h are provided at the left side of the 1.5 h data. FIG. 6 presents the percentage portion of cells in the subG1 area of the cell cycle as determined by the FACS method as mentioned above and below. For achieving an evaluation of the significance a fourfold chi square test is performed. Data show a high significance (p<0.01) for incubation times of about 3 h and above. This high significance is marked by a superscript “a” in the Figure. The data obtained for an incubation time of about 1.5 h show significance (p<0.05) and are marked by a superscript “b” in the Figure.

FIG. 7 shows the decrease of vitality of A375 melanoma cells as given by the Comparative Examples 8 to 10 and Examples 67 to 75. The concentrations of the peptides are provided in the Figure and are 100 μM, 200 μM and 500 μM, respectively. The incubation time is about 24 hours. As shown in FIG. 7, the bars are arranged in groups depicting the concentration and the peptides, respectively. From the bottom to the top the data provided the Peptides SEQ C1, SEQID NO 27, SEQID NO 39 and SEQID NO 38. Furthermore, the data at a concentration of 100 μM are depicted on the bottom for each peptide, while the data at a concentration of 500 μM are depicted on the top for each peptide. The data obtained at a concentration of 200 μM are provided in the middle of each peptide. FIG. 7 presents the percentage portion of surviving cells based on the percentage of cells in the region R-1 in the diagram (application Forward Scatter versus Sideward Scatter) as determined by the FACS method as mentioned above and below. The data achieved considers the data as measured for the solvent alone (DMSO). For achieving an evaluation of the significance a fourfold chi square test is performed. The data show a high significance (p<0.01) for all of the peptides evaluated. This high significance is marked by a superscript “a” in the Figure. As shown in FIG. 7, the peptides of the invention (SEQID NO 27, SEQID NO 39 and SEQID NO 38) provide a significantly greater decrease than the peptide of the prior art (Peptide SEQ C1).

FIG. 8 shows the decrease of vitality of BxPC-3 human pancreatic cancer cells as given by the Comparative Examples 11 to 13 and Examples 76 to 87. The concentrations of the peptides are provided in the Figure and are 50 μM, 100 μM and 200 μM, respectively. The incubation time is about 24 hours. As shown in FIG. 8, the bars are arranged in groups depicting the concentration and the peptides, respectively. From the top to the bottom the data provided the Peptides SEQ C1, SEQID NO 27, SEQID NO 29, SEQID NO 36 and SEQID NO 37. Furthermore, the data at a concentration of 50 μM are depicted on the bottom for each peptide, while the data at a concentration of 200 μM are depicted on the top for each peptide. The data obtained at a concentration of 100 μM are provided in the middle of each peptide. FIG. 8 presents the percentage portion of surviving cells based on the percentage of cells in the region R-1 in the diagram (application Forward Scatter versus Sideward Scatter) as determined by the FACS method as mentioned above and below. For achieving an evaluation of the significance a fourfold chi square test is performed. The data show a high significance (p<0.01) for all of the peptides evaluated. This high significance is marked by a superscript “a” in the Figure. As shown in FIG. 8, the peptides of the invention (SEQID NO 27, SEQID NO 37, SEQID NO 36 and SEQID NO 29) provide a significantly greater decrease than the control peptide (C1).

FIG. 9 shows the decrease of vitality of BxPC-3 human pancreatic cancer cells as given by the Comparative Example 14 and Examples 88 to 94. The concentration of the peptides is 100 μM and the incubation time is about 24 hours. From the bottom to the top the data provided concern the Peptides SEQ C1, SEQID NO 27, SEQID NO 25, SEQID NO 30, SEQID NO 31, SEQID NO 32, SEQID NO 33 and SEQID NO 34. FIG. 9 presents the percentage portion of surviving cells based on the percentage of cells in the region R-1 in the diagram (application Forward Scatter versus Sideward Scatter) as determined by the FACS method as mentioned above and below. The data achieved considers the data as measured for the solvent alone (DMSO). For achieving an evaluation of the significance a fourfold chi square test is performed. The data show a high significance (p<0.01) for all of the peptides evaluated. This high significance is marked by a superscript “*” in the Figure.

FIG. 10 shows the induced cell death caused by apoptosis of BxPC-3 human pancreatic cancer cells as given by the Comparative Examples 15 to 17 and Examples 95 to 106. The concentrations of the peptides are provided in the Figure and are 50 μM, 100 μM and 200 μM, respectively. As shown in FIG. 10, the bars are arranged in groups depicting the concentration and the peptides, respectively. From the top to the bottom the data provided the Peptides SEQ C1, SEQID NO 27, SEQID NO 29, SEQID NO 36 and SEQID NO 37. Furthermore, the data at a concentration of 50 μM are depicted on the bottom for each peptide, while the data at a concentration of 200 μM are depicted on the top for each peptide. The data obtained at a concentration of 100 μM are provided in the middle of each peptide. The incubation time is about 24 hours. FIG. 10 presents the percentage portion of cells in the subG1 area of the cell cycle as determined by the FACS method as mentioned above and below. The data achieved considers the data as measured for the solvent alone (DMSO). For achieving an evaluation of the significance a fourfold chi square test is performed. The data show a high significance (p<0.01) for a comparison of Peptide SEQ Cl versus peptide SEQID NO 27 at 50 μM, 100 μM and 200 μM, respectively. This high significance is marked by a superscript “a” in the Figure. The data show a high significance (p<0.01) for a comparison of Peptide SEQ Cl versus Peptide SEQID No 36 at 50 μM, 100 μM and 200 μM, respectively. This high significance is marked by a superscript “b” in the Figure. The data show a high significance (p<0.01) for a comparison of Peptide SEQ Cl versus Peptide SEQID No 29 at 50 μM, 100 μM and 200 μM, respectively. This high significance is marked by a superscript “b” in the Figure. The data show a high significance (p<0.01) for a comparison of Peptide SEQ Cl versus Peptide SEQID No 37 at 100 μM and 200 μM, respectively. This high significance is marked by a superscript “c” in the Figure.

FIG. 11 shows the decrease of vitality of human B-chronic lymphocytic leukaemia cells (MEC-1) and human peripheral blood mononuclear cells (Lymphocyte, -PBMC″) as given by the Examples 111 to 152, respectively. The concentrations of the peptides are provided in the Figure and are 50 μM, 100 μM and 200 μM, respectively. As shown in FIG. 11, the bars are arranged in groups depicting the cell type, the concentration and the peptides, respectively. From the left to the right side the data provided the peptides SEQID NO 27, SEQID NO 25, SEQID NO 26, SEQID NO 30, SEQID NO 31, SEQID NO 32 and SEQID NO 33. The Figure depicts the data for each peptide by six bars. The three bars on the left side of each peptide concerns the data for the MEC-1 cancer cells (bars having stripes) while the three bars on the right side of each peptide concerns the data obtained with healthy Lymphocyte (PBMC cells; filled bars). Furthermore, the data at a concentration of 50 μM are depicted on the left side for each peptide and each cell type, while the data at a concentration of 200 μM are depicted on the right side for each peptide and each cell type. For cell type and each peptide the data at a concentration of 100 μM are provided in the middle. The incubation time is about 2 hours. FIG. 11 presents the percentage portion of surviving cells based on the percentage of cells in the region R-1 in the diagram (application Forward Scatter versus Sideward Scatter) as determined by the FACS method as mentioned above and below. The data achieved considers the data as measured for the solvent alone (DMSO).

FIG. 12 shows the decrease of vitality of human breast carcinoma cells (MDA-MB-231) as given by the Examples 153 to 170, respectively. The concentrations of the peptides are provided in the Figure and are 50 μM, 100 μM and 200 μM, respectively. As shown in FIG. 12, the bars are arranged in groups depicting the peptides, the concentration and the treatment time, respectively. From the left to the right side the data provided the peptides SEQID NO 36, SEQID NO 37 and SEQID NO 27. The Figure depicts the data for each peptide by six bars. The bars on the left side of each peptide concerns the data for a concentration of 200 μM while the bars on the right side of each peptide concerns the data obtained for a concentration of 50 μM. The data obtained for a concentration of 100 μM are depicted in the middle of the data for each peptide. Furthermore, the data obtained with a treatment time of 48 h are depicted on the left side for each peptide and each concentration (filled bars), while the data obtained with a treatment time of 24h are depicted on the right side for each peptide and each concentration (bars having stripes). FIG. 12 presents the percentage portion of surviving cells based on the respiration activity method as mentioned above and below. The data achieved considers the data as measured for the solvent alone (DMSO).

FIG. 13 shows the induced cell death caused by apoptosis of human breast carcinoma cells (MDA-MB-231). The concentrations of all peptides used are 200 μM. The incubation time is about 24 hours. FIG. 13 presents the percentage portion of cells in the subG1 area of the cell cycle as determined by the FACS method as mentioned above. The apoptosis data achieved considers the data as measured for the solvent alone (DMSO). For achieving an evaluation of the significance a fourfold chi square test is performed. The data show a high significance (p<0.01) for all of the peptides evaluated compared to comparative peptide C1.

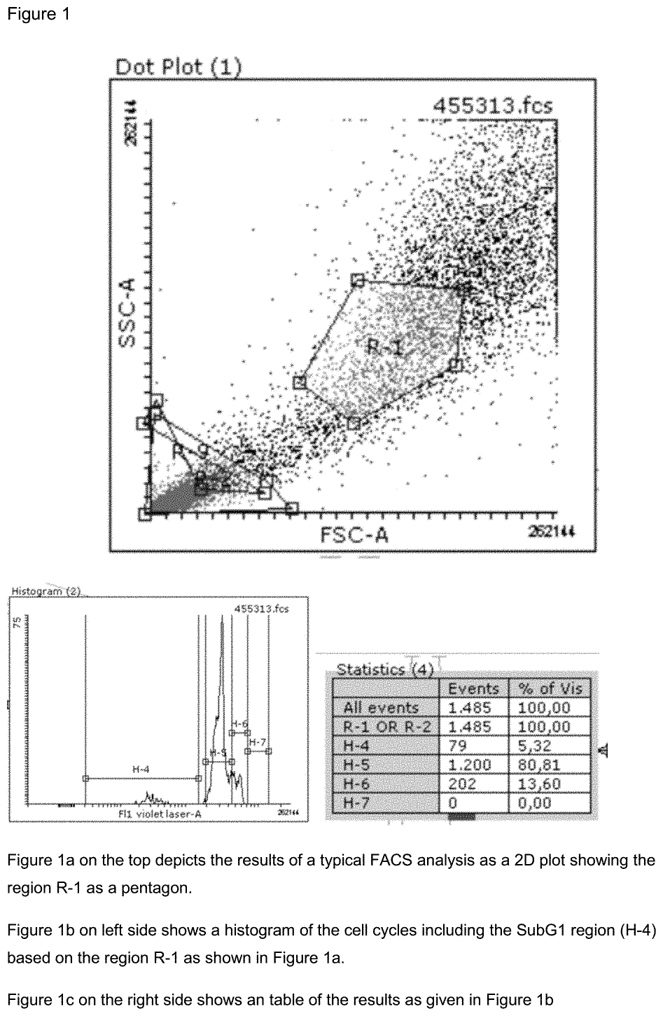

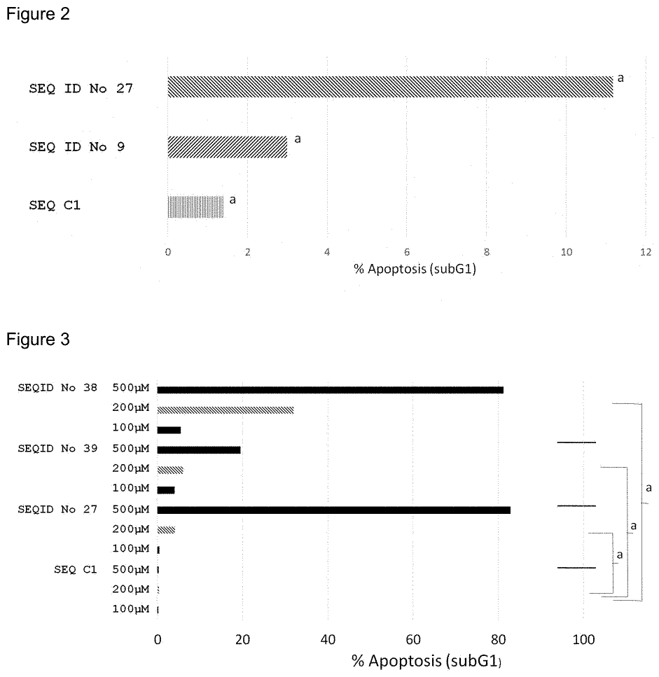

Addition of one amino acid at the carboxy terminus (SEQ No 3 and SEQ No 4) or at the amino terminus (SEQ No 1 and SEQ No 2) leads to an increased apoptosis-inducing activity compared to the peptide SEQ C1. Adding one additional nonpolar amino acid to peptide SEQID No 1 leading to SEQID No 12 causes a slightly decreased activity compared to peptide SEQID No 1. Nevertheless, peptide SEQID No 12 shows a remarkably improved efficiency than SEQ C1. However, the improvement of SEQID No 12 is smaller than the improvement of peptide SEQID No 1. As shown in FIG. 2, adding one additional polar amino acid to peptide SEQID No 1 leading to peptide SEQID No 9 causes a extraordinary increased activity compared to peptide SEQ C1.

FIG. 14 shows the induced cell death caused by apoptosis of BxPC-3 pancreas cancer cells. The concentrations of all peptides used was 100 μM. The incubation time is about 24 hours. FIG. 14 presents the percentage portion of cells in the subG1 area of the cell cycle as determined by the FACS method as mentioned above. The apoptosis data achieved considers the data as measured for the solvent alone (DMSO). For achieving an evaluation of the significance a fourfold chi square test is performed. The data show a high significance (p<0.01) for all of the peptides evaluated compared to comparative peptide C1.

Adding one amino acid (SEQID No 1, -2 and -3) or two amino acids (SEQID No 9 and -12) leads to a stronger apoptosis induction compared to SEQ C1. Adding one additional polar amino acid to peptide SEQID No 1 leading to SEQID No 9 causes an strongly improved activity compared to peptide SEQID No 1.

FIG. 15 shows the induced cell death caused by apoptosis of human breast carcinoma cells (MDA-MB-231). The concentrations of all peptides used was 100 μM. The incubation time is about 24 hours. FIG. 15 presents the percentage portion of cells in the subG1 area of the cell cycle as determined by the FACS method as mentioned above. The apoptosis data achieved considers the data as measured for the solvent alone (DMSO). An exchange of the amino-terminal amino acid from k in SEQID No 27 to q in SEQID No 28 leads to a higher apoptosis-inducing activity. For achieving an evaluation of the significance a fourfold chi square test is performed. The data show a high significance (p<0.01) for the peptide SEQID No 27 evaluated compared to peptide SEQID No 28.

FIG. 16 shows the induced cell death caused by apoptosis of human breast carcinoma cells (MDA-MB-231). The concentrations of the peptides used was 100 μM. The incubation time is about 24 hours. FIG. 16 presents the percentage portion of cells in the subG1 area of the cell cycle as determined by the FACS method as mentioned above. The apoptosis data achieved considers the data as measured for the solvent alone (DMSO). An exchange of one amino acid from k to q at position 4 of peptide SEQID No 33 results in peptide SEQID No 40 which shows a similar activity in the subG1 apoptosis assay system measuring the effect to MDA-MB-231 cells. In FIG. 16 SEQID 40 shows a slightly smaller efficiency than SEQID No 33. However, an evaluation of the significance a fourfold chi square test is performed. The data show a low significance (p>0.1) for the peptide SEQID No 33 evaluated compared to peptide SEQID No 40.

FIG. 17 shows the induced cell death caused by apoptosis of BxPC-3 pancreas cancer cells. The concentrations of the peptides used was 100 μM. The incubation time is about 24 hours. FIG. 17 presents the percentage portion of cells in the subG1 area of the cell cycle as determined by the FACS method as mentioned above. The apoptosis data achieved considers the data as measured for the solvent alone (DMSO). For achieving an evaluation of the significance a fourfold chi square test is performed. The data show a high significance (p<0.01) for the peptide SEQID No 33 evaluated compared to peptide SEQID No 40.

An exchange of one amino acid from k to q at position 4 of peptide SEQID No 33 results in peptide SEQID No 40 which shows a weaker activity in the subG1 apoptosis assay system measuring the effect to BxPC-3 cells.

FIG. 18 shows the induced cell death caused by apoptosis of human breast carcinoma cells (MDA-MB-231). The concentrations of peptides SEQID No 28 and SEQID No 1 used was 100 μM, the concentration of the peptide SEQID No 41 which contains two active peptide motifs with the sequence mvvyfr used was 50 μM. The incubation time is about 24 hours. FIG. 18 presents the percentage portion of cells in the subG1 area of the cell cycle as determined by the FACS method as mentioned above. The apoptosis data achieved considers the data as measured for the solvent alone (DMSO). For achieving an evaluation of the significance a fourfold chi square test is performed. The data show a high significance (p<0.01) for the peptide SEQID No 41 evaluated compared to peptide SEQID No 28. However, the data show a low significance (p>0.1) for the peptide SEQID No 1 evaluated compared to peptide SEQID No 28.

The dimeric peptide which is applied at half the concentration of the monomeric peptides, thereby resulting in an identical concentration of the active peptide motif, yields a more than two-fold apoptosis-inducing activity than the peptides SEQID No 28 and SEQID No 1. FIG. 18 clearly shows that a peptide having two, three, four or more amino acid sequences m-v-v-y-f-r (first peptide portion) have a superior efficiency. Based on the fact that the molar concentration of the binding motif mvvyfr are the same in the examples, the use of a peptide having two, three, four or more amino acid sequences m-v-v-y-f-r (first peptide portion) provides a synergistic effect.

FIG. 19 shows the induced cell death caused by apoptosis of BxPC-3 pancreas cancer cells. The concentrations of peptides SEQID No 28 and SEQID No 27 used was 100 μM, the concentration of the peptide peptide SEQID No 41 which contains two active peptide motifs with the sequence mvvyfr used was 50 μM. The incubation time is about 24 hours. FIG. 19 presents the percentage portion of cells in the subG1 area of the cell cycle as determined by the FACS method as mentioned above. The apoptosis data achieved considers the data as measured for the solvent alone (DMSO). For achieving an evaluation of the significance a fourfold chi square test is performed. The data show a very high significance (p<0.001) for the peptide SEQID No 41 evaluated compared to peptide SEQID No 28. Additionally, the data show a high significance (p<0.01) for the peptide SEQID No 27 evaluated compared to peptide SEQID No 28.

The dimeric peptide which is applied at half the concentration of the monomeric peptides, thereby resulting in an identical concentration of the active peptide motif, yields a more than two-fold apoptosis-inducing activity than the peptides SEQID No 28 and SEQID No 27.

### COMPARATIVE EXAMPLE 1

As mentioned above A375 melanoma cells are cultivated, harvested and evaluated using the Protocol B. The apoptosis efficiency of Peptide SEQ Cl is measured using the subG1 method mentioned above at a concentration of the peptide of about 100 μM and an incubation time of about 24 hours. The data obtained are depicted FIG. 2.

The Comparative Example 1 has been repeated for an incubation time of about 24 hours and 6 hours and shows essentially the same results.

### Example 1

As mentioned above A375 melanoma cells are cultivated, harvested and evaluated using the Protocol B. The apoptosis efficiency of peptide SEQID No 9 is measured using the subG1 method mentioned above at a concentration of the peptide of about 100 μM and an incubation time of about 24 hours. The data obtained are depicted FIG. 2.

### Example 2

As mentioned above A375 melanoma cells are cultivated, harvested and evaluated using the Protocol B. The apoptosis efficiency of peptide SEQID NO 27 is measured using the subG1 method mentioned above at a concentration of the peptide of about 100 μM and an incubation time of about 24 hours. The data obtained are depicted FIG. 2.

The Example 2 has been repeated for an incubation time of about 24 hours and 6 hours and shows essentially the same results.

The Examples 1 and 2 and the Comparative Example 1 have been performed using cells being obtained with one cultivation approach which has been split up for the tests.

The evaluation of Comparative Example 1 and Examples 1 and 2 clearly shows an astonishing improvement of the apoptosis efficiency of the inventive pharmaceutically active substances. Especially Example 2 demonstrates an at least sevenfold efficiency of peptide SEQID NO 27 in view of Peptide SEQ C1.

### COMPARATIVE EXAMPLE 2

As mentioned above A375 melanoma cells are cultivated, harvested and evaluated using the Protocol A. The apoptosis efficiency of Peptide SEQ Cl is measured using the subG1 method mentioned above at a concentration of the peptide of about 100 OA and an incubation time of about 24 hours. The data obtained are depicted FIG. 3.

### COMPARATIVE EXAMPLE 3

As mentioned above A375 melanoma cells are cultivated, harvested and evaluated using the Protocol A. The apoptosis efficiency of Peptide SEQ Cl is measured using the subG1 method mentioned above at a concentration of the peptide of about 200 OA and an incubation time of about 24 hours. The data obtained are depicted FIG. 3.

### COMPARATIVE EXAMPLE 4

As mentioned above A375 melanoma cells are cultivated, harvested and evaluated using the Protocol A. The apoptosis efficiency of Peptide SEQ Cl is measured using the subG1 method mentioned above at a concentration of the peptide of about 500 OA and an incubation time of about 24 hours. The data obtained are depicted FIG. 3.

### Example 3

As mentioned above A375 melanoma cells are cultivated, harvested and evaluated using the Protocol A. The apoptosis efficiency of peptide SEQID NO 27 is measured using the subG1 method mentioned above at a concentration of the peptide of about 100 OA and an incubation time of about 24 hours. The data obtained are depicted FIG. 3.

### Example 4

As mentioned above A375 melanoma cells are cultivated, harvested and evaluated using the Protocol A. The apoptosis efficiency of peptide SEQID NO 27 is measured using the subG1 method mentioned above at a concentration of the peptide of about 200 OA and an incubation time of about 24 hours. The data obtained are depicted FIG. 3.

### Example 5

As mentioned above A375 melanoma cells are cultivated, harvested and evaluated using the Protocol A. The apoptosis efficiency of peptide SEQID NO 27 is measured using the subG1 method mentioned above at a concentration of the peptide of about 500 OA and an incubation time of about 24 hours. The data obtained are depicted FIG. 3.

### Example 6

As mentioned above A375 melanoma cells are cultivated, harvested and evaluated using the Protocol A. The apoptosis efficiency of Peptide SEQID NO 39 is measured using the subG1 method mentioned above at a concentration of the peptide of about 100 OA and an incubation time of about 24 hours. The data obtained are depicted FIG. 3.

### Example 7

As mentioned above A375 melanoma cells are cultivated, harvested and evaluated using the Protocol A. The apoptosis efficiency of Peptide SEQID NO 39 is measured using the subG1 method mentioned above at a concentration of the peptide of about 200 OA and an incubation time of about 24 hours. The data obtained are depicted FIG. 3.

### Example 8

As mentioned above A375 melanoma cells are cultivated, harvested and evaluated using the Protocol A. The apoptosis efficiency of Peptide SEQID NO 39 is measured using the subG1 method mentioned above at a concentration of the peptide of about 500 OA and an incubation time of about 24 hours. The data obtained are depicted FIG. 3.

### Example 9

As mentioned above A375 melanoma cells are cultivated, harvested and evaluated using the Protocol A. The apoptosis efficiency of Peptide SEQID No 38 is measured using the subG1 method mentioned above at a concentration of the peptide of about 100 OA and an incubation time of about 24 hours. The data obtained are depicted FIG. 3.

### Example 10

As mentioned above A375 melanoma cells are cultivated, harvested and evaluated using the Protocol A. The apoptosis efficiency of Peptide SEQID No 38 is measured using the subG1 method mentioned above at a concentration of the peptide of about 200 OA and an incubation time of about 24 hours. The data obtained are depicted FIG. 3.

### Example 11

As mentioned above A375 melanoma cells are cultivated, harvested and evaluated using the Protocol A. The apoptosis efficiency of Peptide SEQID No 38 is measured using the subG1 method mentioned above at a concentration of the peptide of about 500 μM and an incubation time of about 24 hours. The data obtained are depicted FIG. 3.

The Examples 3 to 11 and the Comparative Examples 2 to 4 have been performed using cells being obtained with one cultivation approach which has been split up for the tests.

The evaluation of Comparative Examples 2 to 4 and Examples 3 to 11 clearly shows an astonishing improvement of the apoptosis efficiency of the inventive pharmaceutically active substances. Especially Examples 9 to 11 demonstrate an at least tenfold efficiency of Peptide SEQID No 38 in view of Peptide SEQ Cl at any concentration. Additionally, the Peptide SEQID No 38 provides a clear improvement over peptide SEQID NO 27 at a concentration below 500 μM and over Peptide SEQID NO 39 at a concentration above 100 μM.

### COMPARATIVE EXAMPLE 5

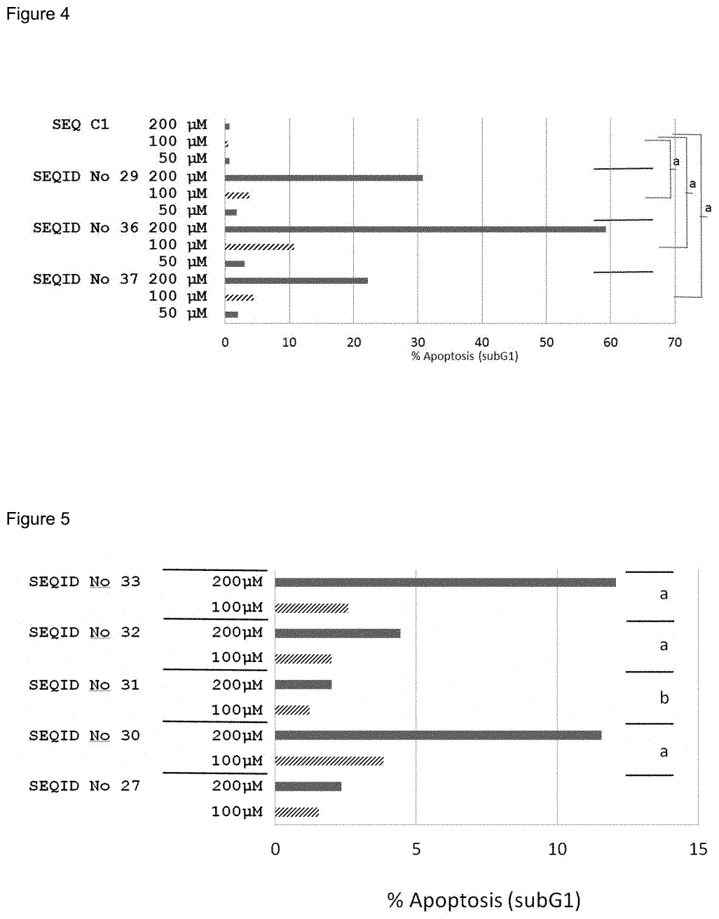

As mentioned above A375 melanoma cells are cultivated, harvested and evaluated using the Protocol A. The apoptosis efficiency of Peptide SEQ Cl is measured using the subG1 method mentioned above at a concentration of the peptide of about 50 μM and an incubation time of about 24 hours. The data obtained are depicted FIG. 4.

### COMPARATIVE EXAMPLE 6

As mentioned above A375 melanoma cells are cultivated, harvested and evaluated using the Protocol A. The apoptosis efficiency of Peptide SEQ Cl is measured using the subG1 method mentioned above at a concentration of the peptide of about 100 μM and an incubation time of about 24 hours. The data obtained are depicted FIG. 4.

### COMPARATIVE EXAMPLE 7

As mentioned above A375 melanoma cells are cultivated, harvested and evaluated using the Protocol A. The apoptosis efficiency of Peptide SEQ Cl is measured using the subG1 method mentioned above at a concentration of the peptide of about 200 μM and an incubation time of about 24 hours. The data obtained are depicted FIG. 4.

### Example 12

As mentioned above A375 melanoma cells are cultivated, harvested and evaluated using the Protocol A. The apoptosis efficiency of Peptide SEQID No 37 is measured using the subG1 method mentioned above at a concentration of the peptide of about 50 OA and an incubation time of about 24 hours. The data obtained are depicted FIG. 4.

### Example 13

As mentioned above A375 melanoma cells are cultivated, harvested and evaluated using the Protocol A. The apoptosis efficiency of Peptide SEQID No 37 is measured using the subG1 method mentioned above at a concentration of the peptide of about 100 OA and an incubation time of about 24 hours. The data obtained are depicted FIG. 4.

### Example 14

As mentioned above A375 melanoma cells are cultivated, harvested and evaluated using the Protocol A. The apoptosis efficiency of Peptide SEQID No 37 is measured using the subG1 method mentioned above at a concentration of the peptide of about 200 OA and an incubation time of about 24 hours. The data obtained are depicted FIG. 4.

### Example 15

As mentioned above A375 melanoma cells are cultivated, harvested and evaluated using the Protocol A. The apoptosis efficiency of Peptide SEQID No 36 is measured using the subG1 method mentioned above at a concentration of the peptide of about 50 OA and an incubation time of about 24 hours. The data obtained are depicted FIG. 4.

### Example 16

As mentioned above A375 melanoma cells are cultivated, harvested and evaluated using the Protocol A. The apoptosis efficiency of Peptide SEQID No 36 is measured using the subG1 method mentioned above at a concentration of the peptide of about 100 OA and an incubation time of about 24 hours. The data obtained are depicted FIG. 4.

### Example 17

As mentioned above A375 melanoma cells are cultivated, harvested and evaluated using the Protocol A. The apoptosis efficiency of Peptide SEQID No 36 is measured using the subG1 method mentioned above at a concentration of the peptide of about 200 OA and an incubation time of about 24 hours. The data obtained are depicted FIG. 4.

### Example 18

As mentioned above A375 melanoma cells are cultivated, harvested and evaluated using the Protocol A. The apoptosis efficiency of Peptide SEQID No 29 is measured using the subG1 method mentioned above at a concentration of the peptide of about 50 OA and an incubation time of about 24 hours. The data obtained are depicted FIG. 4.

### Example 19

As mentioned above A375 melanoma cells are cultivated, harvested and evaluated using the Protocol A. The apoptosis efficiency of Peptide SEQID No 29 is measured using the subG1 method mentioned above at a concentration of the peptide of about 100 μM and an incubation time of about 24 hours. The data obtained are depicted FIG. 4.

### Example 20

As mentioned above A375 melanoma cells are cultivated, harvested and evaluated using the Protocol A. The apoptosis efficiency of Peptide SEQID No 29 is measured using the subG1 method mentioned above at a concentration of the peptide of about 200 μM and an incubation time of about 24 hours. The data obtained are depicted FIG. 4.

The Examples 12 to 20 and the Comparative Examples 5 to 7 have been performed using cells being obtained with one cultivation approach which has been split up for the tests.

The evaluation of Comparative Example 5 and Examples 12 to 20 clearly shows an astonishing improvement of the apoptosis efficiency of the inventive pharmaceutically active substances. Especially Examples 15 to 17 demonstrate at 100 μM an at least fivefold efficiency and at 200 μM an at least tenfold efficiency of Peptide SEQID No 36 in view of Peptide SEQ C1. Additionally, the Peptide SEQID No 36 provides a clear improvement over Peptides SEQID NO 37 and SEQID NO 29 at any concentration. Furthermore, the Peptide SEQID No 29 shows higher efficiency at 200 μM in view of Peptide SEQID No 37.

### Example 21

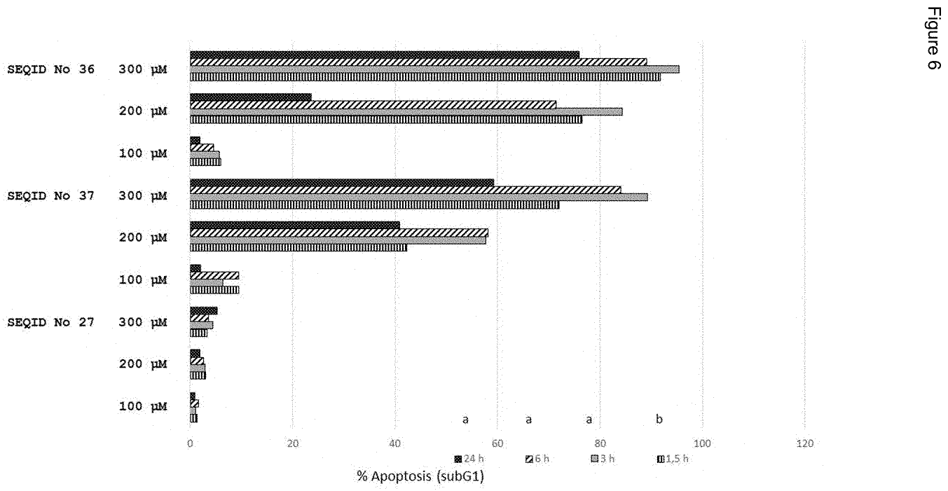

As mentioned above A375 melanoma cells are cultivated, harvested and evaluated using the Protocol A. The apoptosis efficiency of peptide SEQID NO 27 is measured using the subG1 method mentioned above at a concentration of the peptide of about 100 μM and an incubation time of about 24 hours. The data obtained are depicted FIG. 5.

### Example 22

As mentioned above A375 melanoma cells are cultivated, harvested and evaluated using the Protocol A. The apoptosis efficiency of peptide SEQID NO 27 is measured using the subG1 method mentioned above at a concentration of the peptide of about 200 μM and an incubation time of about 24 hours. The data obtained are depicted FIG. 5.

### Example 23

As mentioned above A375 melanoma cells are cultivated, harvested and evaluated using the Protocol A. The apoptosis efficiency of Peptide SEQID NO 30 is measured using the subG1 method mentioned above at a concentration of the peptide of about 100 OA and an incubation time of about 24 hours. The data obtained are depicted FIG. 5.

### Example 24

As mentioned above A375 melanoma cells are cultivated, harvested and evaluated using the Protocol A. The apoptosis efficiency of Peptide SEQID No 30 is measured using the subG1 method mentioned above at a concentration of the peptide of about 200 OA and an incubation time of about 24 hours. The data obtained are depicted FIG. 5.

### Example 25

As mentioned above A375 melanoma cells are cultivated, harvested and evaluated using the Protocol A. The apoptosis efficiency of Peptide SEQID No 31 is measured using the subG1 method mentioned above at a concentration of the peptide of about 100 OA and an incubation time of about 24 hours. The data obtained are depicted FIG. 5.

### Example 26

As mentioned above A375 melanoma cells are cultivated, harvested and evaluated using the Protocol A. The apoptosis efficiency of Peptide SEQID No 31 is measured using the subG1 method mentioned above at a concentration of the peptide of about 200 OA and an incubation time of about 24 hours. The data obtained are depicted FIG. 5.

### Example 27

As mentioned above A375 melanoma cells are cultivated, harvested and evaluated using the Protocol A. The apoptosis efficiency of Peptide SEQID No 32 is measured using the subG1 method mentioned above at a concentration of the peptide of about 100 OA and an incubation time of about 24 hours. The data obtained are depicted FIG. 5.

### Example 28

As mentioned above A375 melanoma cells are cultivated, harvested and evaluated using the Protocol A. The apoptosis efficiency of Peptide SEQID No 32 is measured using the subG1 method mentioned above at a concentration of the peptide of about 200 OA and an incubation time of about 24 hours. The data obtained are depicted FIG. 5.

### Example 29

As mentioned above A375 melanoma cells are cultivated, harvested and evaluated using the Protocol A. The apoptosis efficiency of Peptide SEQID No 33 is measured using the subG1 method mentioned above at a concentration of the peptide of about 100 OA and an incubation time of about 24 hours. The data obtained are depicted FIG. 5.

### Example 30

As mentioned above A375 melanoma cells are cultivated, harvested and evaluated using the Protocol A. The apoptosis efficiency of Peptide SEQID No 33 is measured using the subG1 method mentioned above at a concentration of the peptide of about 200 μM and an incubation time of about 24 hours. The data obtained are depicted FIG. 5.

The Examples 21 to 30 have been performed using cells being obtained with one cultivation approach which has been split up for the tests.

The evaluation of Examples 21 to 30 clearly shows a high apoptosis efficiency of the inventive pharmaceutically active substances. At a concentration of 200 μM the Peptides SEQID No 30 and SEQID NO 33 exhibit an improvement over the peptides SEQID NO 27, SEQID NO 31 and SEQID NO 32, while the Peptide SEQID No 32 provides a higher efficiency than peptides SEQID NO 27 and SEQID NO 31. Furthermore, the Peptide SEQID No 30 shows higher efficiency at 100 μM in view of peptides SEQID NO 27 and SEQID NO 31. In addition thereto, the Peptides SEQID No 31 and SEQID NO 32 have an improved apoptosis level in view of peptide SEQID NO 27.

### Example 31

As mentioned above A375 melanoma cells are cultivated, harvested and evaluated using the Protocol A. The apoptosis efficiency of peptide SEQID NO 27 is measured using the subG1 method mentioned above at a concentration of the peptide of about 100 μM and an incubation time of about 1.5 hours. The data obtained are depicted FIG. 6.

### Example 32

As mentioned above A375 melanoma cells are cultivated, harvested and evaluated using the Protocol A. The apoptosis efficiency of peptide SEQID NO 27 is measured using the subG1 method mentioned above at a concentration of the peptide of about 100 μM and an incubation time of about 3 hours. The data obtained are depicted FIG. 6.

### Example 33

As mentioned above A375 melanoma cells are cultivated, harvested and evaluated using the Protocol A. The apoptosis efficiency of peptide SEQID NO 27 is measured using the subG1 method mentioned above at a concentration of the peptide of about 100 μM and an incubation time of about 6 hours. The data obtained are depicted FIG. 6.

### Example 34

As mentioned above A375 melanoma cells are cultivated, harvested and evaluated using the Protocol A. The apoptosis efficiency of peptide SEQID NO 27 is measured using the subG1 method mentioned above at a concentration of the peptide of about 100 OA and an incubation time of about 24 hours. The data obtained are depicted FIG. 6.

### Example 35

As mentioned above A375 melanoma cells are cultivated, harvested and evaluated using the Protocol A. The apoptosis efficiency of peptide SEQID NO 27 is measured using the subG1 method mentioned above at a concentration of the peptide of about 200 OA and an incubation time of about 1.5 hours. The data obtained are depicted FIG. 6.

### Example 36

As mentioned above A375 melanoma cells are cultivated, harvested and evaluated using the Protocol A. The apoptosis efficiency of peptide SEQID NO 27 is measured using the subG1 method mentioned above at a concentration of the peptide of about 200 OA and an incubation time of about 3 hours. The data obtained are depicted FIG. 6.

### Example 37

As mentioned above A375 melanoma cells are cultivated, harvested and evaluated using the Protocol A. The apoptosis efficiency of peptide SEQID NO 27 is measured using the subG1 method mentioned above at a concentration of the peptide of about 200 OA and an incubation time of about 6 hours. The data obtained are depicted FIG. 6.

### Example 38

As mentioned above A375 melanoma cells are cultivated, harvested and evaluated using the Protocol A. The apoptosis efficiency of peptide SEQID NO 27 is measured using the subG1 method mentioned above at a concentration of the peptide of about 200 OA and an incubation time of about 24 hours. The data obtained are depicted FIG. 6.

### Example 39

As mentioned above A375 melanoma cells are cultivated, harvested and evaluated using the Protocol A. The apoptosis efficiency of peptide SEQID NO 27 is measured using the subG1 method mentioned above at a concentration of the peptide of about 300 OA and an incubation time of about 1.5 hours. The data obtained are depicted FIG. 6.

### Example 40

As mentioned above A375 melanoma cells are cultivated, harvested and evaluated using the Protocol A. The apoptosis efficiency of peptide SEQID NO 27 is measured using the subG1 method mentioned above at a concentration of the peptide of about 300 OA and an incubation time of about 3 hours. The data obtained are depicted FIG. 6.

### Example 41

As mentioned above A375 melanoma cells are cultivated, harvested and evaluated using the Protocol A. The apoptosis efficiency of peptide SEQID NO 27 is measured using the subG1 method mentioned above at a concentration of the peptide of about 300 OA and an incubation time of about 6 hours. The data obtained are depicted FIG. 6.

### Example 42

As mentioned above A375 melanoma cells are cultivated, harvested and evaluated using the Protocol A. The apoptosis efficiency of peptide SEQID NO 27 is measured using the subG1 method mentioned above at a concentration of the peptide of about 300 OA and an incubation time of about 24 hours. The data obtained are depicted FIG. 6.

### Example 43

As mentioned above A375 melanoma cells are cultivated, harvested and evaluated using the Protocol A. The apoptosis efficiency of Peptide SEQID No 37 is measured using the subG1 method mentioned above at a concentration of the peptide of about 100 OA and an incubation time of about 1.5 hours. The data obtained are depicted FIG. 6.

### Example 44

As mentioned above A375 melanoma cells are cultivated, harvested and evaluated using the Protocol A. The apoptosis efficiency of Peptide SEQID No 37 is measured using the subG1 method mentioned above at a concentration of the peptide of about 100 OA and an incubation time of about 3 hours. The data obtained are depicted FIG. 6.

### Example 45

As mentioned above A375 melanoma cells are cultivated, harvested and evaluated using the Protocol A. The apoptosis efficiency of Peptide SEQID No 37 is measured using the subG1 method mentioned above at a concentration of the peptide of about 100 OA and an incubation time of about 6 hours. The data obtained are depicted FIG. 6.

### Example 46

As mentioned above A375 melanoma cells are cultivated, harvested and evaluated using the Protocol A. The apoptosis efficiency of Peptide SEQID No 37 is measured using the subG1 method mentioned above at a concentration of the peptide of about 100 OA and an incubation time of about 24 hours. The data obtained are depicted FIG. 6.

### Example 47

As mentioned above A375 melanoma cells are cultivated, harvested and evaluated using the Protocol A. The apoptosis efficiency of Peptide SEQID No 37 is measured using the subG1 method mentioned above at a concentration of the peptide of about 200 OA and an incubation time of about 1.5 hours. The data obtained are depicted FIG. 6.

### Example 48

As mentioned above A375 melanoma cells are cultivated, harvested and evaluated using the Protocol A. The apoptosis efficiency of Peptide SEQID No 37 is measured using the subG1 method mentioned above at a concentration of the peptide of about 200 OA and an incubation time of about 3 hours. The data obtained are depicted FIG. 6.

### Example 49

As mentioned above A375 melanoma cells are cultivated, harvested and evaluated using the Protocol A. The apoptosis efficiency of Peptide SEQID No 37 is measured using the subG1 method mentioned above at a concentration of the peptide of about 200 OA and an incubation time of about 6 hours. The data obtained are depicted FIG. 6.

### Example 50

As mentioned above A375 melanoma cells are cultivated, harvested and evaluated using the Protocol A. The apoptosis efficiency of Peptide SEQID No 37 is measured using the subG1 method mentioned above at a concentration of the peptide of about 200 OA and an incubation time of about 24 hours. The data obtained are depicted FIG. 6.

### Example 51

As mentioned above A375 melanoma cells are cultivated, harvested and evaluated using the Protocol A. The apoptosis efficiency of Peptide SEQID No 37 is measured using the subG1 method mentioned above at a concentration of the peptide of about 300 OA and an incubation time of about 1.5 hours. The data obtained are depicted FIG. 6.

### Example 52

As mentioned above A375 melanoma cells are cultivated, harvested and evaluated using the Protocol A. The apoptosis efficiency of Peptide SEQID No 37 is measured using the subG1 method mentioned above at a concentration of the peptide of about 300 OA and an incubation time of about 3 hours. The data obtained are depicted FIG. 6.

### Example 53

As mentioned above A375 melanoma cells are cultivated, harvested and evaluated using the Protocol A. The apoptosis efficiency of Peptide SEQID No 37 is measured using the subG1 method mentioned above at a concentration of the peptide of about 300 OA and an incubation time of about 6 hours. The data obtained are depicted FIG. 6.

### Example 54

As mentioned above A375 melanoma cells are cultivated, harvested and evaluated using the Protocol A. The apoptosis efficiency of Peptide SEQID No 37 is measured using the subG1 method mentioned above at a concentration of the peptide of about 300 OA and an incubation time of about 24 hours. The data obtained are depicted FIG. 6.

### Example 55

As mentioned above A375 melanoma cells are cultivated, harvested and evaluated using the Protocol A. The apoptosis efficiency of Peptide SEQID No 36 is measured using the subG1 method mentioned above at a concentration of the peptide of about 100 OA and an incubation time of about 1.5 hours. The data obtained are depicted FIG. 6.

### Example 56

As mentioned above A375 melanoma cells are cultivated, harvested and evaluated using the Protocol A. The apoptosis efficiency of Peptide SEQID No 36 is measured using the subG1 method mentioned above at a concentration of the peptide of about 100 OA and an incubation time of about 3 hours. The data obtained are depicted FIG. 6.

### Example 57

As mentioned above A375 melanoma cells are cultivated, harvested and evaluated using the Protocol A. The apoptosis efficiency of Peptide SEQID No 36 is measured using the subG1 method mentioned above at a concentration of the peptide of about 100 OA and an incubation time of about 6 hours. The data obtained are depicted FIG. 6.

### Example 58

As mentioned above A375 melanoma cells are cultivated, harvested and evaluated using the Protocol A. The apoptosis efficiency of Peptide SEQID No 36 is measured using the subG1 method mentioned above at a concentration of the peptide of about 100 OA and an incubation time of about 24 hours. The data obtained are depicted FIG. 6.

### Example 59

As mentioned above A375 melanoma cells are cultivated, harvested and evaluated using the Protocol A. The apoptosis efficiency of Peptide SEQID No 36 is measured using the subG1 method mentioned above at a concentration of the peptide of about 200 OA and an incubation time of about 1.5 hours. The data obtained are depicted FIG. 6.

### Example 60

As mentioned above A375 melanoma cells are cultivated, harvested and evaluated using the Protocol A. The apoptosis efficiency of Peptide SEQID No 36 is measured using the subG1 method mentioned above at a concentration of the peptide of about 200 OA and an incubation time of about 3 hours. The data obtained are depicted FIG. 6.

### Example 61

As mentioned above A375 melanoma cells are cultivated, harvested and evaluated using the Protocol A. The apoptosis efficiency of Peptide SEQID No 36 is measured using the subG1 method mentioned above at a concentration of the peptide of about 200 OA and an incubation time of about 6 hours. The data obtained are depicted FIG. 6.

### Example 62

As mentioned above A375 melanoma cells are cultivated, harvested and evaluated using the Protocol A. The apoptosis efficiency of Peptide SEQID No 36 is measured using the subG1 method mentioned above at a concentration of the peptide of about 200 OA and an incubation time of about 24 hours. The data obtained are depicted FIG. 6.

### Example 63

As mentioned above A375 melanoma cells are cultivated, harvested and evaluated using the Protocol A. The apoptosis efficiency of Peptide SEQID No 36 is measured using the subG1 method mentioned above at a concentration of the peptide of about 300 OA and an incubation time of about 1.5 hours. The data obtained are depicted FIG. 6.

### Example 64

As mentioned above A375 melanoma cells are cultivated, harvested and evaluated using the Protocol A. The apoptosis efficiency of Peptide SEQID No 36 is measured using the subG1 method mentioned above at a concentration of the peptide of about 300 OA and an incubation time of about 3 hours. The data obtained are depicted FIG. 6.

### Example 65

As mentioned above A375 melanoma cells are cultivated, harvested and evaluated using the Protocol A. The apoptosis efficiency of Peptide SEQID No 36 is measured using the subG1 method mentioned above at a concentration of the peptide of about 300 OA and an incubation time of about 6 hours. The data obtained are depicted FIG. 6.

### Example 66

As mentioned above A375 melanoma cells are cultivated, harvested and evaluated using the Protocol A. The apoptosis efficiency of Peptide SEQID No 36 is measured using the subG1 method mentioned above at a concentration of the peptide of about 300 OA and an incubation time of about 24 hours. The data obtained are depicted FIG. 6.

The Examples 31 to 66 have been performed using cells being obtained with one cultivation approach which has been split up for the tests.

The evaluation of Examples 31 to 66 clearly shows a high apoptosis efficiency of the inventive pharmaceutically active substances. Please note the high apoptosis level at an incubation time of about 3 hours at a peptide concentration of 200 OA and above regarding the highly efficient Peptide SEQID No 36. It seems that surviving cells are dividing and leading to such result. At a concentration of 300 OA the Peptide SEQID No 36 provides an improvement with regard to all incubation times over Peptides SEQID NO 37 and SEQID NO 27. At a concentration of 200 OA the peptide provides an improvement over Peptides SEQID NO 37 and SEQID NO 27 at incubation times of 1.5 h, 3 h and 6 h while at an incubation time of 24 h the Peptide SEQID No 37 shows the best results. At a concentration of 100 OA the Peptides SEQID NO 37 and SEQID NO 36 show a slightly improved apoptosis level in view of peptide SEQID NO 27.

### COMPARATIVE EXAMPLE 8

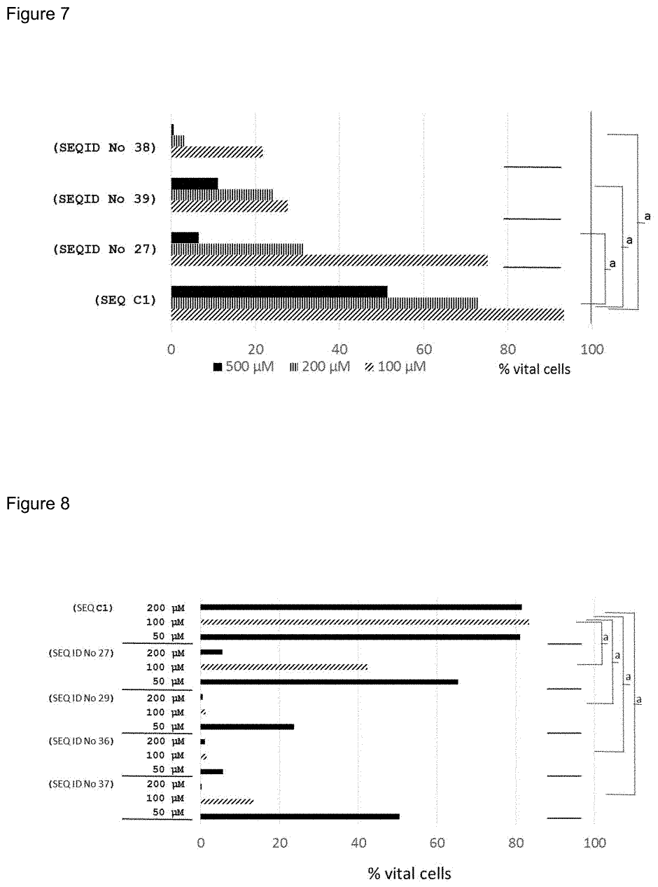

As mentioned above A375 melanoma cells are cultivated, harvested and evaluated using the Protocol A. The apoptosis efficiency of Peptide SEQ Cl is measured using the cell vitality method mentioned above at a concentration of the peptide of about 100 OA and an incubation time of about 24 hours. The data obtained are depicted FIG. 7.

### COMPARATIVE EXAMPLE 9

As mentioned above A375 melanoma cells are cultivated, harvested and evaluated using the Protocol A. The apoptosis efficiency of Peptide SEQ Cl is measured using the cell vitality method mentioned above at a concentration of the peptide of about 200 OA and an incubation time of about 24 hours. The data obtained are depicted FIG. 7.

### COMPARATIVE EXAMPLE 10

As mentioned above A375 melanoma cells are cultivated, harvested and evaluated using the Protocol A. The apoptosis efficiency of Peptide SEQ Cl is measured using the cell vitality method mentioned above at a concentration of the peptide of about 500 OA and an incubation time of about 24 hours. The data obtained are depicted FIG. 7.

### Example 67

As mentioned above A375 melanoma cells are cultivated, harvested and evaluated using the Protocol A. The apoptosis efficiency of peptide SEQID NO 27 is measured using the cell vitality method mentioned above at a concentration of the peptide of about 100 OA and an incubation time of about 24 hours. The data obtained are depicted FIG. 7.

### Example 68

As mentioned above A375 melanoma cells are cultivated, harvested and evaluated using the Protocol A. The apoptosis efficiency of peptide SEQID NO 27 is measured using the cell vitality method mentioned above at a concentration of the peptide of about 200 OA and an incubation time of about 24 hours. The data obtained are depicted FIG. 7.

### Example 69

As mentioned above A375 melanoma cells are cultivated, harvested and evaluated using the Protocol A. The apoptosis efficiency of peptide SEQID NO 27 is measured using the cell vitality method mentioned above at a concentration of the peptide of about 500 OA and an incubation time of about 24 hours. The data obtained are depicted FIG. 7.

### Example 70

As mentioned above A375 melanoma cells are cultivated, harvested and evaluated using the Protocol A. The apoptosis efficiency of Peptide SEQID NO 39 is measured using the cell vitality method mentioned above at a concentration of the peptide of about 100 OA and an incubation time of about 24 hours. The data obtained are depicted FIG. 7.

### Example 71

As mentioned above A375 melanoma cells are cultivated, harvested and evaluated using the Protocol A. The apoptosis efficiency of Peptide SEQID NO 39 is measured using the cell vitality method mentioned above at a concentration of the peptide of about 200 OA and an incubation time of about 24 hours. The data obtained are depicted FIG. 7.

### Example 72

As mentioned above A375 melanoma cells are cultivated, harvested and evaluated using the Protocol A. The apoptosis efficiency of Peptide SEQID NO 39 is measured using the cell vitality method mentioned above at a concentration of the peptide of about 500 μM and an incubation time of about 24 hours. The data obtained are depicted FIG. 7.

### Example 73

As mentioned above A375 melanoma cells are cultivated, harvested and evaluated using the Protocol A. The apoptosis efficiency of Peptide SEQID No 38 is measured using the cell vitality method mentioned above at a concentration of the peptide of about 100 μM and an incubation time of about 24 hours. The data obtained are depicted FIG. 7.

### Example 74

As mentioned above A375 melanoma cells are cultivated, harvested and evaluated using the Protocol A. The apoptosis efficiency of Peptide SEQID No 38 is measured using the cell vitality method mentioned above at a concentration of the peptide of about 200 μM and an incubation time of about 24 hours. The data obtained are depicted FIG. 7.

### Example 75

As mentioned above A375 melanoma cells are cultivated, harvested and evaluated using the Protocol A. The apoptosis efficiency of Peptide SEQID No 38 is measured using the cell vitality method mentioned above at a concentration of the peptide of about 500 μM and an incubation time of about 24 hours. The data obtained are depicted FIG. 7.

The Examples 67 to 75 and the Comparative Examples 8 to 10 have been performed using cells being obtained with one cultivation approach which has been split up for the tests.

The evaluation of Comparative Examples 8 to 10 and Examples 67 to 75 clearly shows an astonishing improvement of the apoptosis efficiency of the inventive pharmaceutically active substances. Especially Examples 73 to 75 demonstrate an at least threefold efficiency of Peptide SEQID No 38 in view of Peptide SEQ Cl at 200 μM, and an at least tenfold efficiency at 500 μM. Additionally, the Peptide SEQID No 38 provides a clear improvement over peptides SEQID NO 27 and SEQID NO 39 at any concentration. Furthermore, the peptide SEQID NO 27 shows higher efficiency at 500 μM in view of peptide SEQID NO 39 while peptide SEQID NO 39 shows a higher apoptosis rate at a concentration of 100 μM as peptide SEQID NO 27.

### COMPARATIVE EXAMPLE 11

As mentioned above BxPC-3 cells are cultivated, harvested and evaluated using the Protocol A. The apoptosis efficiency of Peptide SEQ Cl is measured using the cell vitality method mentioned above at a concentration of the peptide of about 50 OA and an incubation time of about 24 hours. The data obtained are depicted FIG. 8.

### COMPARATIVE EXAMPLE 12

As mentioned above BxPC-3 cells are cultivated, harvested and evaluated using the Protocol A. The apoptosis efficiency of Peptide SEQ Cl is measured using the cell vitality method mentioned above at a concentration of the peptide of about 100 OA and an incubation time of about 24 hours. The data obtained are depicted FIG. 8.

### COMPARATIVE EXAMPLE 13

As mentioned above BxPC-3 cells are cultivated, harvested and evaluated using the Protocol A. The apoptosis efficiency of Peptide SEQ Cl is measured using the cell vitality method mentioned above at a concentration of the peptide of about 200 OA and an incubation time of about 24 hours. The data obtained are depicted FIG. 8.

### Example 76

As mentioned above BxPC-3 cells are cultivated, harvested and evaluated using the Protocol A. The apoptosis efficiency of peptide SEQID NO 27 is measured using the cell vitality method mentioned above at a concentration of the peptide of about 50 OA and an incubation time of about 24 hours. The data obtained are depicted FIG. 8.

### Example 77

As mentioned above BxPC-3 cells are cultivated, harvested and evaluated using the Protocol A. The apoptosis efficiency of peptide SEQID NO 27 is measured using the cell vitality method mentioned above at a concentration of the peptide of about 100 OA and an incubation time of about 24 hours. The data obtained are depicted FIG. 8.

### Example 78

As mentioned above BxPC-3 cells are cultivated, harvested and evaluated using the Protocol A. The apoptosis efficiency of peptide SEQID NO 27 is measured using the cell vitality method mentioned above at a concentration of the peptide of about 200 OA and an incubation time of about 24 hours. The data obtained are depicted FIG. 8.

### Example 79

As mentioned above BxPC-3 cells are cultivated, harvested and evaluated using the Protocol A. The apoptosis efficiency of Peptide SEQID No 37 is measured using the cell vitality method mentioned above at a concentration of the peptide of about 50 OA and an incubation time of about 24 hours. The data obtained are depicted FIG. 8.

### Example 80

As mentioned above BxPC-3 cells are cultivated, harvested and evaluated using the Protocol A. The apoptosis efficiency of Peptide SEQID No 37 is measured using the cell vitality method mentioned above at a concentration of the peptide of about 100 OA and an incubation time of about 24 hours. The data obtained are depicted FIG. 8.

### Example 81

As mentioned above BxPC-3 cells are cultivated, harvested and evaluated using the Protocol A. The apoptosis efficiency of Peptide SEQID No 37 is measured using the cell vitality method mentioned above at a concentration of the peptide of about 200 OA and an incubation time of about 24 hours. The data obtained are depicted FIG. 8.

### Example 82

As mentioned above BxPC-3 cells are cultivated, harvested and evaluated using the Protocol A. The apoptosis efficiency of Peptide SEQID No 36 is measured using the cell vitality method mentioned above at a concentration of the peptide of about 50 OA and an incubation time of about 24 hours. The data obtained are depicted FIG. 8.

### Example 83

As mentioned above BxPC-3 cells are cultivated, harvested and evaluated using the Protocol A. The apoptosis efficiency of Peptide SEQID No 36 is measured using the cell vitality method mentioned above at a concentration of the peptide of about 100 OA and an incubation time of about 24 hours. The data obtained are depicted FIG. 8.

### Example 84

As mentioned above BxPC-3 cells are cultivated, harvested and evaluated using the Protocol A. The apoptosis efficiency of Peptide SEQID No 36 is measured using the cell vitality method mentioned above at a concentration of the peptide of about 200 OA and an incubation time of about 24 hours. The data obtained are depicted FIG. 8.

### Example 85

As mentioned above BxPC-3 cells are cultivated, harvested and evaluated using the Protocol A. The apoptosis efficiency of Peptide SEQID No 29 is measured using the cell vitality method mentioned above at a concentration of the peptide of about 50 μM and an incubation time of about 24 hours. The data obtained are depicted FIG. 8.

### Example 86

As mentioned above BxPC-3 cells are cultivated, harvested and evaluated using the Protocol A. The apoptosis efficiency of Peptide SEQID No 29 is measured using the cell vitality method mentioned above at a concentration of the peptide of about 100 μM and an incubation time of about 24 hours. The data obtained are depicted FIG. 8.

### Example 87

As mentioned above BxPC-3 cells are cultivated, harvested and evaluated using the Protocol A. The apoptosis efficiency of Peptide SEQID No 29 is measured using the cell vitality method mentioned above at a concentration of the peptide of about 200 μM and an incubation time of about 24 hours. The data obtained are depicted FIG. 8.

The Examples 76 to 87 and the Comparative Examples 11 to 13 have been performed using cells being obtained with one cultivation approach which has been split up for the tests.

The evaluation of Comparative Examples 11 to 13 and Examples 76 to 87 clearly shows an astonishing improvement of the apoptosis efficiency of the inventive pharmaceutically active substances. Especially Examples 82 to 84 demonstrate an at least fourfold efficiency of Peptide SEQID No 36 in view of Peptide SEQ Cl at 50 μM concentration, and an at least sevenfold efficiency of Peptide SEQID No 36 in view of Peptide SEQ Cl at 100 and 200 μM concentration. Additionally, the Peptide SEQID No 36 provides a clear improvement over peptides SEQID NO 27, SEQID NO 37 and SEQID NO 29 at a concentration of 50 μM. At a concentration of 100 μM the Peptides SEQID No 36 and SEQID NO 29 exhibit a very high efficiency. At a concentration of 200 μM the Peptides SEQID NO 37, SEQID NO 36 and SEQID NO 29 exhibits an astonishing high efficiency. Furthermore, the peptide SEQID NO 27 shows a lower efficiency at all concentrations than the Peptides SEQID NO 37, SEQID NO 36 and SEQID NO 29.

### COMPARATIVE EXAMPLE 14

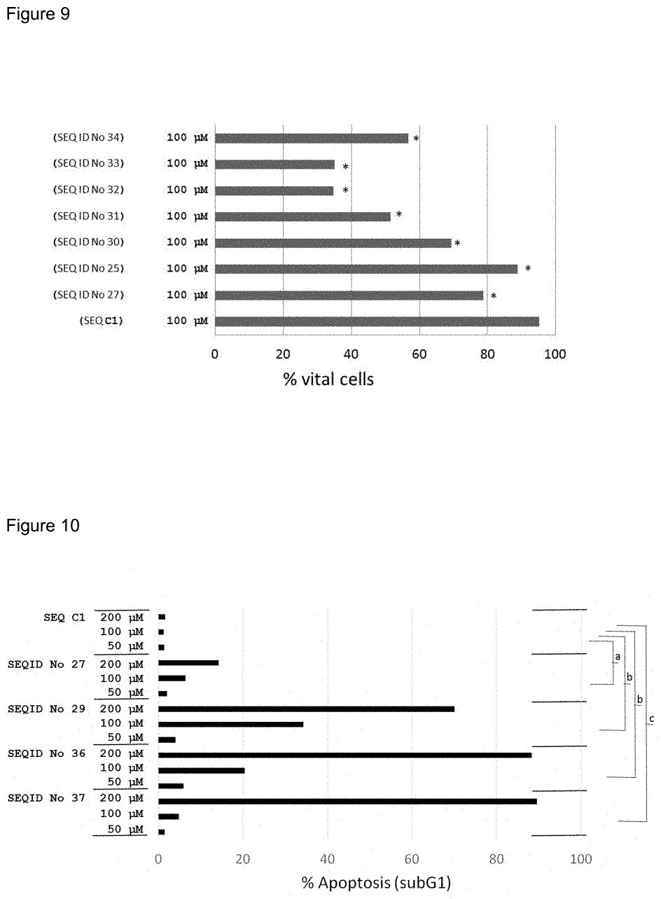

As mentioned above BxPC-3 cells are cultivated, harvested and evaluated using the Protocol B. The apoptosis efficiency of Peptide SEQ Cl is measured using the cell vitality method mentioned above at a concentration of the peptide of about 100 OA and an incubation time of about 24 hours. The data obtained are depicted FIG. 9.

### Example 88

As mentioned above BxPC-3 cells are cultivated, harvested and evaluated using the Protocol B. The apoptosis efficiency of peptide SEQID NO 27 is measured using the cell vitality method mentioned above at a concentration of the peptide of about 100 OA and an incubation time of about 24 hours. The data obtained are depicted FIG. 9.

### Example 89

As mentioned above BxPC-3 cells are cultivated, harvested and evaluated using the Protocol B. The apoptosis efficiency of Peptide SEQID NO 25 is measured using the cell vitality method mentioned above at a concentration of the peptide of about 100 OA and an incubation time of about 24 hours. The data obtained are depicted FIG. 9.

### Example 90

As mentioned above BxPC-3 cells are cultivated, harvested and evaluated using the Protocol B. The apoptosis efficiency of Peptide SEQID No 30 is measured using the cell vitality method mentioned above at a concentration of the peptide of about 100 OA and an incubation time of about 24 hours. The data obtained are depicted FIG. 9.

### Example 91

As mentioned above BxPC-3 cells are cultivated, harvested and evaluated using the Protocol B. The apoptosis efficiency of Peptide SEQID No 31 is measured using the cell vitality method mentioned above at a concentration of the peptide of about 100 OA and an incubation time of about 24 hours. The data obtained are depicted FIG. 9.

### Example 92

As mentioned above BxPC-3 cells are cultivated, harvested and evaluated using the Protocol B. The apoptosis efficiency of Peptide SEQID No 32 is measured using the cell vitality method mentioned above at a concentration of the peptide of about 100 OA and an incubation time of about 24 hours. The data obtained are depicted FIG. 9.

### Example 93

As mentioned above BxPC-3 cells are cultivated, harvested and evaluated using the Protocol B. The apoptosis efficiency of Peptide SEQID No 33 is measured using the cell vitality method mentioned above at a concentration of the peptide of about 100 OA and an incubation time of about 24 hours. The data obtained are depicted FIG. 9.

### Example 94

As mentioned above BxPC-3 cells are cultivated, harvested and evaluated using the Protocol B. The apoptosis efficiency of Peptide SEQID No 34 is measured using the cell vitality method mentioned above at a concentration of the peptide of about 100 OA and an incubation time of about 24 hours. The data obtained are depicted FIG. 9.

The Examples 88 to 94 and the Comparative Example 14 have been performed using cells being obtained with one cultivation approach which has been split up for the tests.

The evaluation of Comparative Example 14 and Examples 88 to and 94 clearly shows an astonishing improvement of the apoptosis efficiency of the inventive pharmaceutically active substances. Regarding the inventive pharmaceutically active substances, the Peptide SEQID NO 25 shows the lowest level of apoptosis while the Peptides SEQID No 32 and SEQID NO 33 induce the highest level of apoptosis.

### COMPARATIVE EXAMPLE 15

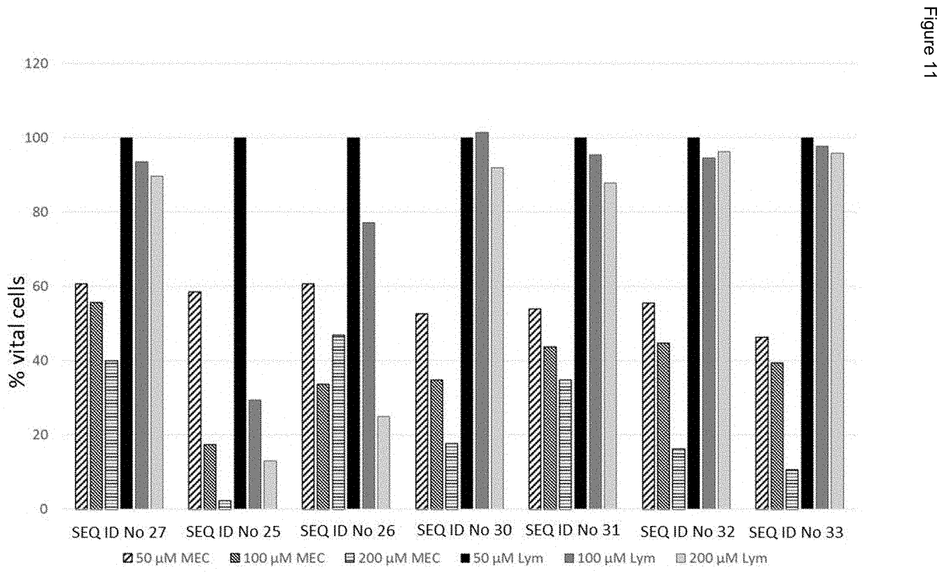

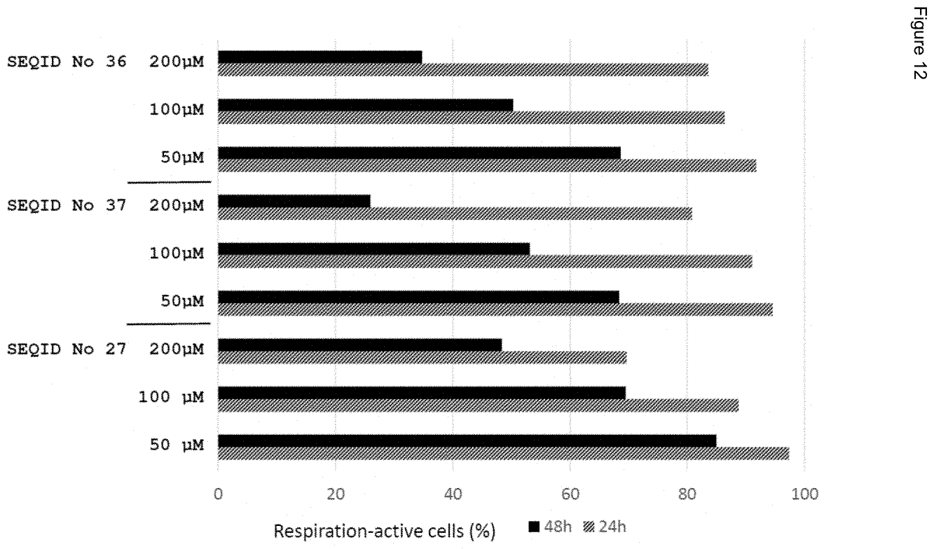

As mentioned above BxPC-3 cells are cultivated, harvested and evaluated using the Protocol A. The apoptosis efficiency of Peptide SEQ Cl is measured using the subG1 method mentioned above at a concentration of the peptide of about 50 OA and an incubation time of about 24 hours. The data obtained are depicted FIG. 10.

### COMPARATIVE EXAMPLE 16

As mentioned above BxPC-3 cells are cultivated, harvested and evaluated using the Protocol A. The apoptosis efficiency of Peptide SEQ Cl is measured using the subG1 method mentioned above at a concentration of the peptide of about 100 OA and an incubation time of about 24 hours. The data obtained are depicted FIG. 10.

### COMPARATIVE EXAMPLE 17

As mentioned above BxPC-3 cells are cultivated, harvested and evaluated using the Protocol A. The apoptosis efficiency of Peptide SEQ Cl is measured using the subG1 method mentioned above at a concentration of the peptide of about 200 OA and an incubation time of about 24 hours. The data obtained are depicted FIG. 10.

### Example 95

As mentioned above BxPC-3 cells are cultivated, harvested and evaluated using the Protocol A. The apoptosis efficiency of peptide SEQID NO 27 is measured using the subG1 method mentioned above at a concentration of the peptide of about 50 OA and an incubation time of about 24 hours. The data obtained are depicted FIG. 10.

### Example 96

As mentioned above BxPC-3 cells are cultivated, harvested and evaluated using the Protocol A. The apoptosis efficiency of peptide SEQID NO 27 is measured using the subG1 method mentioned above at a concentration of the peptide of about 100 OA and an incubation time of about 24 hours. The data obtained are depicted FIG. 10.

### Example 97

As mentioned above BxPC-3 cells are cultivated, harvested and evaluated using the Protocol A. The apoptosis efficiency of peptide SEQID NO 27 is measured using the subG1 method mentioned above at a concentration of the peptide of about 200 OA and an incubation time of about 24 hours. The data obtained are depicted FIG. 10.

### Example 98

As mentioned above BxPC-3 cells are cultivated, harvested and evaluated using the Protocol A. The apoptosis efficiency of Peptide SEQID No 37 is measured using the subG1 method mentioned above at a concentration of the peptide of about 50 OA and an incubation time of about 24 hours. The data obtained are depicted FIG. 10.

### Example 99

As mentioned above BxPC-3 cells are cultivated, harvested and evaluated using the Protocol A. The apoptosis efficiency of Peptide SEQID No 37 is measured using the subG1 method mentioned above at a concentration of the peptide of about 100 OA and an incubation time of about 24 hours. The data obtained are depicted FIG. 10.

### Example 100

As mentioned above BxPC-3 cells are cultivated, harvested and evaluated using the Protocol A. The apoptosis efficiency of Peptide SEQID No 37 is measured using the subG1 method mentioned above at a concentration of the peptide of about 200 OA and an incubation time of about 24 hours. The data obtained are depicted FIG. 10.

### Example 101

As mentioned above BxPC-3 cells are cultivated, harvested and evaluated using the Protocol A. The apoptosis efficiency of Peptide SEQID No 36 is measured using the subG1 method mentioned above at a concentration of the peptide of about 50 OA and an incubation time of about 24 hours. The data obtained are depicted FIG. 10.

### Example 102

As mentioned above BxPC-3 cells are cultivated, harvested and evaluated using the Protocol A. The apoptosis efficiency of Peptide SEQID No 36 is measured using the subG1 method mentioned above at a concentration of the peptide of about 100 OA and an incubation time of about 24 hours. The data obtained are depicted FIG. 10.

### Example 103

As mentioned above BxPC-3 cells are cultivated, harvested and evaluated using the Protocol A. The apoptosis efficiency of Peptide SEQID No 36 is measured using the subG1 method mentioned above at a concentration of the peptide of about 200 OA and an incubation time of about 24 hours. The data obtained are depicted FIG. 10.

### Example 104

As mentioned above BxPC-3 cells are cultivated, harvested and evaluated using the Protocol A. The apoptosis efficiency of Peptide SEQID No 29 is measured using the subG1 method mentioned above at a concentration of the peptide of about 50 OA and an incubation time of about 24 hours. The data obtained are depicted FIG. 10.

### Example 105

As mentioned above BxPC-3 cells are cultivated, harvested and evaluated using the Protocol A. The apoptosis efficiency of Peptide SEQID No 29 is measured using the subG1 method mentioned above at a concentration of the peptide of about 100 OA and an incubation time of about 24 hours. The data obtained are depicted FIG. 10.

### Example 106

As mentioned above BxPC-3 cells are cultivated, harvested and evaluated using the Protocol A. The apoptosis efficiency of Peptide SEQID No 29 is measured using the subG1 method mentioned above at a concentration of the peptide of about 200 OA and an incubation time of about 24 hours. The data obtained are depicted FIG. 10.

The Examples 95 to 106 and the Comparative Examples 15 to 17 have been performed using cells being obtained with one cultivation approach which has been split up for the tests.

The evaluation of Comparative Examples 15 to 17 and Examples 95 to 106 clearly shows an astonishing improvement of the apoptosis efficiency of the inventive pharmaceutically active substances. Especially Examples 101 to 103 demonstrate an at least fourfold efficiency of Peptide SEQID No 36 in view of Peptide SEQ Cl at any concentration. Additionally, the Peptides SEQID NO 37 and SEQID NO 36 provide a clear improvement over peptides SEQID NO 27 and SEQID NO 29 at a concentration of 200 μM. Furthermore, the Peptide SEQID No 29 shows higher efficiency at 100 μM in view of Peptides SEQID NO 27, SEQID NO 37 and SEQID NO 36. At a concentration of 50 μM the Peptides SEQID No 36 and SEQID NO 29 provide a slight improvement over peptides SEQID NO 27 and SEQID NO 37.

### Example 107

As mentioned above BxPC-3 cells are cultivated, harvested and evaluated using the Protocol B. The apoptosis efficiency of Peptide SEQID No 33 is measured using the subG1 method mentioned above at a concentration of the peptide of about 100 μM and an incubation time of about 24 hours.

### Example 108

As mentioned above BxPC-3 cells are cultivated, harvested and evaluated using the Protocol B. The apoptosis efficiency of Peptide SEQID No 33 is measured using the subG1 method mentioned above at a concentration of the peptide of about 200 μM and an incubation time of about 24 hours.

### Example 109

As mentioned above BxPC-3 cells are cultivated, harvested and evaluated using the Protocol B. The apoptosis efficiency of Peptide SEQID NO 35 is measured using the subG1 method mentioned above at a concentration of the peptide of about 100 μM and an incubation time of about 24 hours.

### Example 110

As mentioned above BxPC-3 cells are cultivated, harvested and evaluated using the Protocol B. The apoptosis efficiency of Peptide SEQID NO 35 is measured using the subG1 method mentioned above at a concentration of the peptide of about 200 μM and an incubation time of about 24 hours.

The data obtained in Examples 109 to 110 show that both peptides impart a considerable efficiency to induce apoptosis in pancreatic cancer cells. The peptide sequence SEQID NO 35 provides a slightly higher apoptosis than the peptide sequence SEQID NO 33.

### Example 111

As mentioned above MEC-1 cells are cultivated, harvested and evaluated using the Protocol A. The apoptosis efficiency of peptide SEQID NO 27 is measured using the cell vitality method mentioned above at a concentration of the peptide of about 50 μM and an incubation time of about 24 hours. The data obtained are depicted FIG. 11.

### Example 112

As mentioned above MEC-1 cells are cultivated, harvested and evaluated using the Protocol A. The apoptosis efficiency of peptide SEQID NO 27 is measured using the cell vitality method mentioned above at a concentration of the peptide of about 100 μM and an incubation time of about 24 hours. The data obtained are depicted FIG. 11.

### Example 113

As mentioned above MEC-1 cells are cultivated, harvested and evaluated using the Protocol A. The apoptosis efficiency of peptide SEQID NO 27 is measured using the cell vitality method mentioned above at a concentration of the peptide of about 200 μM and an incubation time of about 24 hours. The data obtained are depicted FIG. 11.

### Example 114

As mentioned above healthy Lymphocyte (PBMC) cells are cultivated, harvested and evaluated using the Protocol A. The apoptosis efficiency of peptide SEQID NO 27 is measured using the cell vitality method mentioned above at a concentration of the peptide of about 50 μM and an incubation time of about 24 hours. The data obtained are depicted FIG. 11.

### Example 115

As mentioned above healthy Lymphocyte (PBMC) cells are cultivated, harvested and evaluated using the Protocol A. The apoptosis efficiency of peptide SEQID NO 27 is measured using the cell vitality method mentioned above at a concentration of the peptide of about 100 μM and an incubation time of about 24 hours. The data obtained are depicted FIG. 11.

### Example 116

As mentioned above healthy Lymphocyte (PBMC) cells are cultivated, harvested and evaluated using the Protocol A. The apoptosis efficiency of peptide SEQID NO 27 is measured using the cell vitality method mentioned above at a concentration of the peptide of about 200 μM and an incubation time of about 24 hours. The data obtained are depicted FIG. 11.

### Example 117

As mentioned above MEC-1 cells are cultivated, harvested and evaluated using the Protocol A. The apoptosis efficiency of Peptide SEQID NO 25 is measured using the cell vitality method mentioned above at a concentration of the peptide of about 50 μM and an incubation time of about 24 hours. The data obtained are depicted FIG. 11.

### Example 118

As mentioned above MEC-1 cells are cultivated, harvested and evaluated using the Protocol A. The apoptosis efficiency of Peptide SEQID NO 25 is measured using the cell vitality method mentioned above at a concentration of the peptide of about 100 μM and an incubation time of about 24 hours. The data obtained are depicted FIG. 11.

### Example 119

As mentioned above MEC-1 cells are cultivated, harvested and evaluated using the Protocol A. The apoptosis efficiency of Peptide SEQID NO 25 is measured using the cell vitality method mentioned above at a concentration of the peptide of about 200 μM and an incubation time of about 24 hours. The data obtained are depicted FIG. 11.

### Example 120

As mentioned above healthy Lymphocyte (PBMC) cells are cultivated, harvested and evaluated using the Protocol A. The apoptosis efficiency of Peptide SEQID NO 25 is measured using the cell vitality method mentioned above at a concentration of the peptide of about 50 μM and an incubation time of about 24 hours. The data obtained are depicted FIG. 11.

### Example 121

As mentioned above healthy Lymphocyte (PBMC) cells are cultivated, harvested and evaluated using the Protocol A. The apoptosis efficiency of Peptide SEQID NO 25 is measured using the cell vitality method mentioned above at a concentration of the peptide of about 100 μM and an incubation time of about 24 hours. The data obtained are depicted FIG. 11.

### Example 122

As mentioned above healthy Lymphocyte (PBMC) cells are cultivated, harvested and evaluated using the Protocol A. The apoptosis efficiency of Peptide SEQID NO 25 is measured using the cell vitality method mentioned above at a concentration of the peptide of about 200 μM and an incubation time of about 24 hours. The data obtained are depicted FIG. 11.

### Example 123

As mentioned above MEC-1 cells are cultivated, harvested and evaluated using the Protocol A. The apoptosis efficiency of Peptide SEQID No 26 is measured using the cell vitality method mentioned above at a concentration of the peptide of about 50 μM and an incubation time of about 24 hours. The data obtained are depicted FIG. 11.

### Example 124

As mentioned above MEC-1 cells are cultivated, harvested and evaluated using the Protocol A. The apoptosis efficiency of Peptide SEQID No 26 is measured using the cell vitality method mentioned above at a concentration of the peptide of about 100 μM and an incubation time of about 24 hours. The data obtained are depicted FIG. 11.

### Example 125

As mentioned above MEC-1 cells are cultivated, harvested and evaluated using the Protocol A. The apoptosis efficiency of Peptide SEQID No 26 is measured using the cell vitality method mentioned above at a concentration of the peptide of about 200 μM and an incubation time of about 24 hours. The data obtained are depicted FIG. 11.

### Example 126

As mentioned above healthy Lymphocyte (PBMC) cells are cultivated, harvested and evaluated using the Protocol A. The apoptosis efficiency of Peptide SEQID No 26 is measured using the cell vitality method mentioned above at a concentration of the peptide of about 50 μM and an incubation time of about 24 hours. The data obtained are depicted FIG. 11.

### Example 127

As mentioned above healthy Lymphocyte (PBMC) cells are cultivated, harvested and evaluated using the Protocol A. The apoptosis efficiency of Peptide SEQID No 26 is measured using the cell vitality method mentioned above at a concentration of the peptide of about 100 μM and an incubation time of about 24 hours. The data obtained are depicted FIG. 11.

### Example 128

As mentioned above healthy Lymphocyte (PBMC) cells are cultivated, harvested and evaluated using the Protocol A. The apoptosis efficiency of Peptide SEQID No 26 is measured using the cell vitality method mentioned above at a concentration of the peptide of about 200 μM and an incubation time of about 24 hours. The data obtained are depicted FIG. 11.

### Example 129

As mentioned above MEC-1 cells are cultivated, harvested and evaluated using the Protocol A. The apoptosis efficiency of Peptide SEQID No 30 is measured using the cell vitality method mentioned above at a concentration of the peptide of about 50 μM and an incubation time of about 24 hours. The data obtained are depicted FIG. 11.

### Example 130

As mentioned above MEC-1 cells are cultivated, harvested and evaluated using the Protocol A. The apoptosis efficiency of Peptide SEQID No 30 is measured using the cell vitality method mentioned above at a concentration of the peptide of about 100 μM and an incubation time of about 24 hours. The data obtained are depicted FIG. 11.

### Example 131

As mentioned above MEC-1 cells are cultivated, harvested and evaluated using the Protocol A. The apoptosis efficiency of Peptide SEQID No 30 is measured using the cell vitality method mentioned above at a concentration of the peptide of about 200 μM and an incubation time of about 24 hours. The data obtained are depicted FIG. 11.

### Example 132

As mentioned above healthy Lymphocyte (PBMC) cells are cultivated, harvested and evaluated using the Protocol A. The apoptosis efficiency of Peptide SEQID No 30 is measured using the cell vitality method mentioned above at a concentration of the peptide of about 50 μM and an incubation time of about 24 hours. The data obtained are depicted FIG. 11.

### Example 133

As mentioned above healthy Lymphocyte (PBMC) cells are cultivated, harvested and evaluated using the Protocol A. The apoptosis efficiency of Peptide SEQID No 30 is measured using the cell vitality method mentioned above at a concentration of the peptide of about 100 μM and an incubation time of about 24 hours. The data obtained are depicted FIG. 11.

### Example 134

As mentioned above healthy Lymphocyte (PBMC) cells are cultivated, harvested and evaluated using the Protocol A. The apoptosis efficiency of Peptide SEQID No 30 is measured using the cell vitality method mentioned above at a concentration of the peptide of about 200 μM and an incubation time of about 24 hours. The data obtained are depicted FIG. 11.

### Example 135

As mentioned above MEC-1 cells are cultivated, harvested and evaluated using the Protocol A. The apoptosis efficiency of Peptide SEQID No 31 is measured using the cell vitality method mentioned above at a concentration of the peptide of about 50 μM and an incubation time of about 24 hours. The data obtained are depicted FIG. 11.

### Example 136

As mentioned above MEC-1 cells are cultivated, harvested and evaluated using the Protocol A. The apoptosis efficiency of Peptide SEQID No 31 is measured using the cell vitality method mentioned above at a concentration of the peptide of about 100 μM and an incubation time of about 24 hours. The data obtained are depicted FIG. 11.

### Example 137

As mentioned above MEC-1 cells are cultivated, harvested and evaluated using the Protocol A. The apoptosis efficiency of Peptide SEQID No 31 is measured using the cell vitality method mentioned above at a concentration of the peptide of about 200 μM and an incubation time of about 24 hours. The data obtained are depicted FIG. 11.

### Example 138

As mentioned above healthy Lymphocyte (PBMC) cells are cultivated, harvested and evaluated using the Protocol A. The apoptosis efficiency of Peptide SEQID No 31 is measured using the cell vitality method mentioned above at a concentration of the peptide of about 50 μM and an incubation time of about 24 hours. The data obtained are depicted FIG. 11.

### Example 139

As mentioned above healthy Lymphocyte (PBMC) cells are cultivated, harvested and evaluated using the Protocol A. The apoptosis efficiency of Peptide SEQID No 31 is measured using the cell vitality method mentioned above at a concentration of the peptide of about 100 μM and an incubation time of about 24 hours. The data obtained are depicted FIG. 11.

### Example 140

As mentioned above healthy Lymphocyte (PBMC) cells are cultivated, harvested and evaluated using the Protocol A. The apoptosis efficiency of Peptide SEQID No 31 is measured using the cell vitality method mentioned above at a concentration of the peptide of about 200 μM and an incubation time of about 24 hours. The data obtained are depicted FIG. 11.

### Example 141

As mentioned above MEC-1 cells are cultivated, harvested and evaluated using the Protocol A. The apoptosis efficiency of Peptide SEQID No 32 is measured using the cell vitality method mentioned above at a concentration of the peptide of about 50 μM and an incubation time of about 24 hours. The data obtained are depicted FIG. 11.

### Example 142

As mentioned above MEC-1 cells are cultivated, harvested and evaluated using the Protocol A. The apoptosis efficiency of Peptide SEQID No 32 is measured using the cell vitality method mentioned above at a concentration of the peptide of about 100 μM and an incubation time of about 24 hours. The data obtained are depicted FIG. 11.

### Example 143

As mentioned above MEC-1 cells are cultivated, harvested and evaluated using the Protocol A. The apoptosis efficiency of Peptide SEQID No 32 is measured using the cell vitality method mentioned above at a concentration of the peptide of about 200 μM and an incubation time of about 24 hours. The data obtained are depicted FIG. 11.

### Example 144

As mentioned above healthy Lymphocyte (PBMC) cells are cultivated, harvested and evaluated using the Protocol A. The apoptosis efficiency of Peptide SEQID No 32 is measured using the cell vitality method mentioned above at a concentration of the peptide of about 50 μM and an incubation time of about 24 hours. The data obtained are depicted FIG. 11.

### Example 145

As mentioned above healthy Lymphocyte (PBMC) cells are cultivated, harvested and evaluated using the Protocol A. The apoptosis efficiency of Peptide SEQID No 32 is measured using the cell vitality method mentioned above at a concentration of the peptide of about 100 μM and an incubation time of about 24 hours. The data obtained are depicted FIG. 11.

### Example 146

As mentioned above healthy Lymphocyte (PBMC) cells are cultivated, harvested and evaluated using the Protocol A. The apoptosis efficiency of Peptide SEQID No 32 is measured using the cell vitality method mentioned above at a concentration of the peptide of about 200 μM and an incubation time of about 24 hours. The data obtained are depicted FIG. 11.

### Example 147

As mentioned above MEC-1 cells are cultivated, harvested and evaluated using the Protocol A. The apoptosis efficiency of Peptide SEQID No 33 is measured using the cell vitality method mentioned above at a concentration of the peptide of about 50 μM and an incubation time of about 24 hours. The data obtained are depicted FIG. 11.

### Example 148

As mentioned above MEC-1 cells are cultivated, harvested and evaluated using the Protocol A. The apoptosis efficiency of Peptide SEQID No 33 is measured using the cell vitality method mentioned above at a concentration of the peptide of about 100 μM and an incubation time of about 24 hours. The data obtained are depicted FIG. 11.

### Example 149

As mentioned above MEC-1 cells are cultivated, harvested and evaluated using the Protocol A. The apoptosis efficiency of Peptide SEQID No 33 is measured using the cell vitality method mentioned above at a concentration of the peptide of about 200 μM and an incubation time of about 24 hours. The data obtained are depicted FIG. 11.

### Example 150

As mentioned above healthy Lymphocyte (PBMC) cells are cultivated, harvested and evaluated using the Protocol A. The apoptosis efficiency of Peptide SEQID No 33 is measured using the cell vitality method mentioned above at a concentration of the peptide of about 50 μM and an incubation time of about 24 hours. The data obtained are depicted FIG. 11.

### Example 151

As mentioned above healthy Lymphocyte (PBMC) cells are cultivated, harvested and evaluated using the Protocol A. The apoptosis efficiency of Peptide SEQID No 33 is measured using the cell vitality method mentioned above at a concentration of the peptide of about 100 μM and an incubation time of about 24 hours. The data obtained are depicted FIG. 11.

### Example 152

As mentioned above healthy Lymphocyte (PBMC) cells are cultivated, harvested and evaluated using the Protocol A. The apoptosis efficiency of Peptide SEQID No 33 is measured using the cell vitality method mentioned above at a concentration of the peptide of about 200 μM and an incubation time of about 24 hours. The data obtained are depicted FIG. 11.

The Examples 111 to 152 have been performed using cells being obtained with one cultivation approach which has been split up for the tests.

The evaluation of Examples 111 to 152 clearly shows a high apoptosis efficiency of the inventive pharmaceutically active substances with regard to the cancer cells. Furthermore the Examples show that healthy Lymphocyte (PBMC) cells tolerate high levels of the inventive pharmaceutically active substances. That is, based on the pharmaceutical mechanism of action of the peptides cells being not prone to apoptosis are not remarkably damaged by the peptides of the present invention. This is especially true with regard to the peptides SEQID NO 27, SEQID NO 30, SEQID NO 31, SEQID NO 32 and SEQID NO 33. Regarding the depicted data, please consider that the solvent DMSO may have a considerable toxic effect to the cells and, hence, even at low levels a measurable degree of vitality is seen in the Figure.

The Peptides SEQID No 30 and SEQID NO 33 show a very high level of induced apoptosis to the cells, while being well tolerated. That is, these Peptides SEQID No 30 and SEQID NO 33 have an astonishingly high selectivity. This is especially true at concentrations of 100 μM and above. The Peptide SEQID No 32 shows the same efficiency as Peptide SEQID No 30 at a concentration of 200 μM and a higher selectivity (lower effect with regard to healthy cells). The peptide SEQID NO 27 is very good tolerated by the healthy cells (low toxic effect). However, the ability to induce apoptosis is lower than the effect of Peptides SEQID No 30, SEQID NO 31, SEQID NO 32 and SEQID NO 33 at any concentration. The Peptides SEQID NO 25 and SEQID NO 26 are not so well tolerated by the healthy cells than the Peptides SEQID NO 27, SEQID NO 30, SEQID NO 31, SEQID NO 32 and SEQID NO 33

### Example 153

As mentioned above MDA-MB-231 cells are cultivated, harvested and evaluated as mentioned above concerning the concerning the respiration activity (ATP). The apoptosis efficiency of peptide SEQID NO 27 is measured using the cell respiration activity method mentioned above at a concentration of the peptide of about 50 μM and an incubation time of about 24 hours. The data obtained are depicted FIG. 12.

### Example 154

As mentioned above MDA-MB-231 cells are cultivated, harvested and evaluated as mentioned above concerning the concerning the respiration activity (ATP). The apoptosis efficiency of peptide SEQID NO 27 is measured using the cell respiration activity method mentioned above at a concentration of the peptide of about 100 μM and an incubation time of about 24 hours. The data obtained are depicted FIG. 12.

### Example 155

As mentioned above MDA-MB-231 cells are cultivated, harvested and evaluated as mentioned above concerning the concerning the respiration activity (ATP). The apoptosis efficiency of peptide SEQID NO 27 is measured using the cell respiration activity method mentioned above at a concentration of the peptide of about 200 μM and an incubation time of about 24 hours. The data obtained are depicted FIG. 12.

### Example 156

As mentioned above MDA-MB-231 cells are cultivated, harvested and evaluated as mentioned above concerning the concerning the respiration activity (ATP). The apoptosis efficiency of peptide SEQID NO 27 is measured using the cell respiration activity method mentioned above at a concentration of the peptide of about 50 μM and an incubation time of about 48 hours. The data obtained are depicted FIG. 12.

### Example 157

As mentioned above MDA-MB-231 cells are cultivated, harvested and evaluated as mentioned above concerning the concerning the respiration activity (ATP). The apoptosis efficiency of peptide SEQID NO 27 is measured using the cell respiration activity method mentioned above at a concentration of the peptide of about 100 μM and an incubation time of about 48 hours. The data obtained are depicted FIG. 12.

### Example 158

As mentioned above MDA-MB-231 cells are cultivated, harvested and evaluated as mentioned above concerning the concerning the respiration activity (ATP). The apoptosis efficiency of peptide SEQID NO 27 is measured using the cell respiration activity method mentioned above at a concentration of the peptide of about 200 μM and an incubation time of about 48 hours. The data obtained are depicted FIG. 12.

### Example 159

As mentioned above MDA-MB-231 cells are cultivated, harvested and evaluated as mentioned above concerning the concerning the respiration activity (ATP). The apoptosis efficiency of peptide SEQID NO 37 is measured using the cell respiration activity method mentioned above at a concentration of the peptide of about 50 μM and an incubation time of about 24 hours. The data obtained are depicted FIG. 12.

### Example 160

As mentioned above MDA-MB-231 cells are cultivated, harvested and evaluated as mentioned above concerning the concerning the respiration activity (ATP). The apoptosis efficiency of peptide SEQID NO 37 is measured using the cell respiration activity method mentioned above at a concentration of the peptide of about 100 μM and an incubation time of about 24 hours. The data obtained are depicted FIG. 12.

### Example 161

As mentioned above MDA-MB-231 cells are cultivated, harvested and evaluated as mentioned above concerning the concerning the respiration activity (ATP). The apoptosis efficiency of peptide SEQID NO 37 is measured using the cell respiration activity method mentioned above at a concentration of the peptide of about 200 μM and an incubation time of about 24 hours. The data obtained are depicted FIG. 12.

### Example 162

As mentioned above MDA-MB-231 cells are cultivated, harvested and evaluated as mentioned above concerning the concerning the respiration activity (ATP). The apoptosis efficiency of peptide SEQID NO 37 is measured using the cell respiration activity method mentioned above at a concentration of the peptide of about 50 μM and an incubation time of about 48 hours. The data obtained are depicted FIG. 12.

### Example 163

As mentioned above MDA-MB-231 cells are cultivated, harvested and evaluated as mentioned above concerning the concerning the respiration activity (ATP). The apoptosis efficiency of peptide SEQID NO 37 is measured using the cell respiration activity method mentioned above at a concentration of the peptide of about 100 μM and an incubation time of about 48 hours. The data obtained are depicted FIG. 12.

### Example 164

As mentioned above MDA-MB-231 cells are cultivated, harvested and evaluated as mentioned above concerning the concerning the respiration activity (ATP). The apoptosis efficiency of peptide SEQID NO 37 is measured using the cell respiration activity method mentioned above at a concentration of the peptide of about 200 μM and an incubation time of about 48 hours. The data obtained are depicted FIG. 12.

### Example 165

As mentioned above MDA-MB-231 cells are cultivated, harvested and evaluated as mentioned above concerning the concerning the respiration activity (ATP). The apoptosis efficiency of peptide SEQID NO 36 is measured using the cell respiration activity method mentioned above at a concentration of the peptide of about 50 μM and an incubation time of about 24 hours. The data obtained are depicted FIG. 12.

### Example 166

As mentioned above MDA-MB-231 cells are cultivated, harvested and evaluated as mentioned above concerning the concerning the respiration activity (ATP). The apoptosis efficiency of peptide SEQID NO 36 is measured using the cell respiration activity method mentioned above at a concentration of the peptide of about 100 μM and an incubation time of about 24 hours. The data obtained are depicted FIG. 12.

### Example 167

As mentioned above MDA-MB-231 cells are cultivated, harvested and evaluated as mentioned above concerning the concerning the respiration activity (ATP). The apoptosis efficiency of peptide SEQID NO 36 is measured using the cell respiration activity method mentioned above at a concentration of the peptide of about 200 μM and an incubation time of about 24 hours. The data obtained are depicted FIG. 12.

### Example 168

As mentioned above MDA-MB-231 cells are cultivated, harvested and evaluated as mentioned above concerning the concerning the respiration activity (ATP). The apoptosis efficiency of peptide SEQID NO 36 is measured using the cell respiration activity method mentioned above at a concentration of the peptide of about 50 μM and an incubation time of about 48 hours. The data obtained are depicted FIG. 12.

### Example 169

As mentioned above MDA-MB-231 cells are cultivated, harvested and evaluated as mentioned above concerning the concerning the respiration activity (ATP). The apoptosis efficiency of peptide SEQID NO 36 is measured using the cell respiration activity method mentioned above at a concentration of the peptide of about 100 μM and an incubation time of about 48 hours. The data obtained are depicted FIG. 12.

### Example 170

As mentioned above MDA-MB-231 cells are cultivated, harvested and evaluated as mentioned above concerning the concerning the respiration activity (ATP). The apoptosis efficiency of peptide SEQID NO 36 is measured using the cell respiration activity method mentioned above at a concentration of the peptide of about 200 μM and an incubation time of about 48 hours. The data obtained are depicted FIG. 12.

The evaluation of Examples 153 to 170 clearly shows a high apoptosis efficiency of the inventive pharmaceutically active substances with regard to the cancer cells. Please note that the respiration activity method measures the presence of ATP which is an energy source in the cells. At a low cell activity the cells show only a low decrease of ATP and the ATP is rather stable and, hence, although the cells itself are killed by the inventive pharmaceutically active substances at a very high speed as shown above, the decrease measured by ATP concentration shows a delay.

### COMPARATIVE EXAMPLE 18

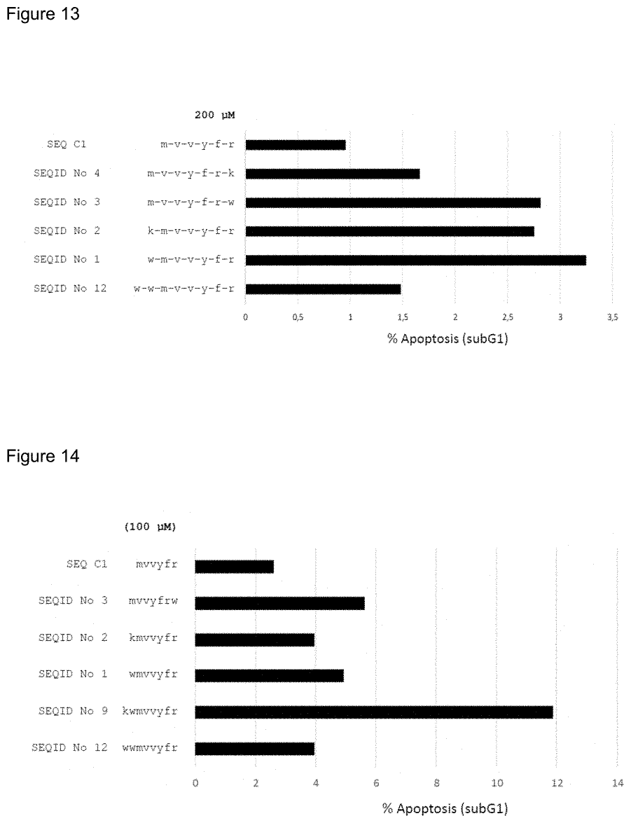

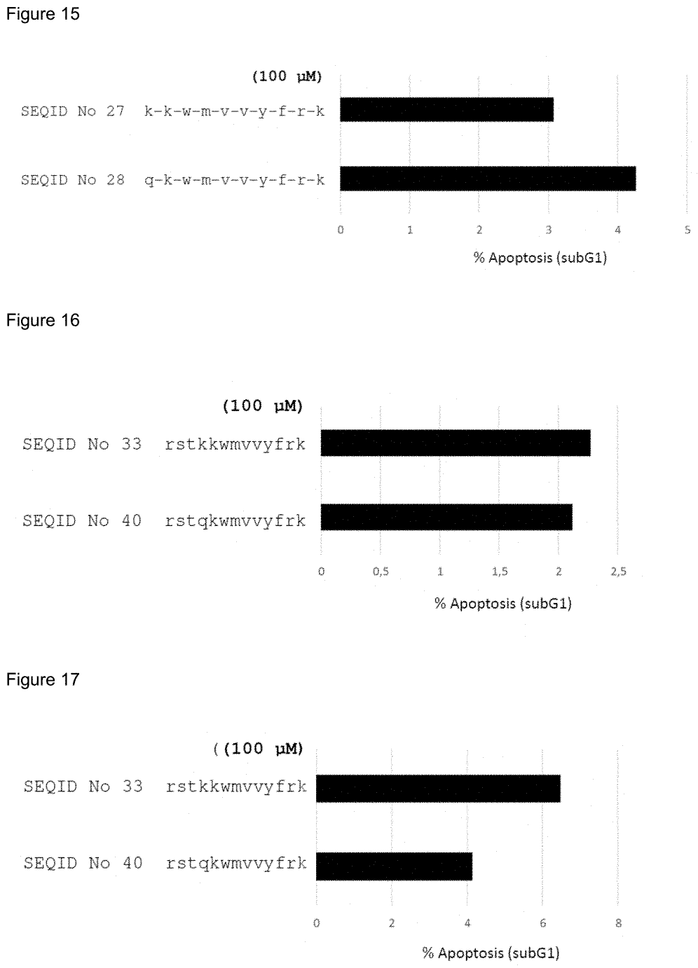

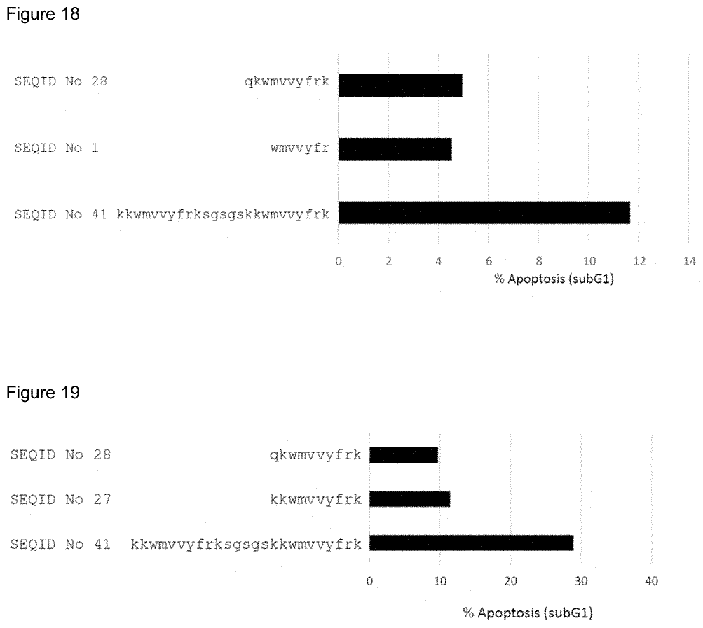

As mentioned above MDA-MB-231 cells are cultivated and harvested. The apoptosis efficiency of Peptide SEQ Cl is measured using the subG1 method mentioned above at a concentration of the peptide of about 200 μM and an incubation time of about 24 hours. The data obtained are depicted FIG. 13.

### Example 171

As mentioned above MDA-MB-231 cells are cultivated and harvested. The apoptosis efficiency of peptide SEQID NO 1 is measured using the subG1 method mentioned above at a concentration of the peptide of about 200 μM and an incubation time of about 24 hours. The data obtained are depicted FIG. 13.

### Example 172

As mentioned above MDA-MB-231 cells are cultivated and harvested. The apoptosis efficiency of peptide SEQID NO 2 is measured using the subG1 method mentioned above at a concentration of the peptide of about 200 μM and an incubation time of about 24 hours. The data obtained are depicted FIG. 13.

### Example 173

As mentioned above MDA-MB-231 cells are cultivated and harvested. The apoptosis efficiency of peptide SEQID NO 3 is measured using the subG1 method mentioned above at a concentration of the peptide of about 200 μM and an incubation time of about 24 hours. The data obtained are depicted FIG. 13.

### Example 174

As mentioned above MDA-MB-231 cells are cultivated and harvested. The apoptosis efficiency of peptide SEQID NO 4 is measured using the subG1 method mentioned above at a concentration of the peptide of about 200 μM and an incubation time of about 24 hours. The data obtained are depicted FIG. 13.

### Example 175

As mentioned above MDA-MB-231 cells are cultivated and harvested. The apoptosis efficiency of peptide SEQID NO 12 is measured using the subG1 method mentioned above at a concentration of the peptide of about 200 OA and an incubation time of about 24 hours. The data obtained are depicted FIG. 13.

The evaluation of Comparative Example 18 and Examples 171 to 175 clearly shows an astonishing improvement of the apoptosis efficiency of the inventive pharmaceutically active substances.

### COMPARATIVE EXAMPLE 19

As mentioned above BxPC-3 cells are cultivated, harvested and evaluated using the Protocol B. The apoptosis efficiency of Peptide SEQ Cl is measured using the subG1 method mentioned above at a concentration of the peptide of about 100 OA and an incubation time of about 24 hours. The data obtained are depicted FIG. 14.

### Example 176

As mentioned above BxPC-3 cells are cultivated, harvested and evaluated using the Protocol B. The apoptosis efficiency of peptide SEQID NO 1 is measured using the subG1 method mentioned above at a concentration of the peptide of about 100 OA and an incubation time of about 24 hours. The data obtained are depicted FIG. 14.

### Example 177

As mentioned above BxPC-3 cells are cultivated, harvested and evaluated using the Protocol B. The apoptosis efficiency of peptide SEQID NO 2 is measured using the subG1 method mentioned above at a concentration of the peptide of about 100 OA and an incubation time of about 24 hours. The data obtained are depicted FIG. 14.

### Example 178

As mentioned above BxPC-3 cells are cultivated, harvested and evaluated using the Protocol B. The apoptosis efficiency of peptide SEQID NO 3 is measured using the subG1 method mentioned above at a concentration of the peptide of about 100 OA and an incubation time of about 24 hours. The data obtained are depicted FIG. 14.

### Example 179

As mentioned above BxPC-3 cells are cultivated, harvested and evaluated using the Protocol B. The apoptosis efficiency of peptide SEQID NO 9 is measured using the subG1 method mentioned above at a concentration of the peptide of about 100 OA and an incubation time of about 24 hours. The data obtained are depicted FIG. 14.

### Example 180

As mentioned above BxPC-3 cells are cultivated, harvested and evaluated using the Protocol B. The apoptosis efficiency of peptide SEQID NO 12 is measured using the subG1 method mentioned above at a concentration of the peptide of about 100 μM and an incubation time of about 24 hours. The data obtained are depicted FIG. 14.

The evaluation of Comparative Example 19 and Examples 176 to 180 clearly shows an astonishing improvement of the apoptosis efficiency of the inventive pharmaceutically active substances. Especially Example 179 demonstrates an at least fourfold efficiency of Peptide SEQID No 9 in view of Peptide SEQ C1. Furthermore, Example 179 demonstrates an at least threefold efficiency of Peptide SEQID No 9 in view of Peptide SEQ No 12. This clearly shows an unexpected improvement of peptides having a polar amino acid, preferably a basic amino acid within a sequence of 5 amino acid units from the first peptide portion. That is, a peptide comprising at least additional one amino acid and/or peptide portion at the N-terminal end and/or at the C-terminal end of the first peptide portion (second peptide portion and/or third peptide portion) being directly bound to the peptide portion having the amino add sequences m-v-v-y-f-r (first peptide portion) wherein the second peptide portion and/or third peptide portion comprising at least one polar amino acid within a sequence of 5 amino acid units from the first peptide portion, preferably within a sequence of 3 amino acid units from the first peptide portion is preferred over a peptide having only nonpolar amino acid within a sequence of 5 amino acid units from the first peptide portion.

### Example 181

As mentioned above MDA-MB-231 cells are cultivated and harvested. The apoptosis efficiency of peptide SEQID NO 27 is measured using the subG1 method mentioned above at a concentration of the peptide of about 100 μM and an incubation time of about 24 hours. The data obtained are depicted FIG. 15.

### Example 182

As mentioned above MDA-MB-231 cells are cultivated and harvested. The apoptosis efficiency of peptide SEQID NO 28 is measured using the subG1 method mentioned above at a concentration of the peptide of about 100 μM and an incubation time of about 24 hours. The data obtained are depicted FIG. 15.

The evaluation of Examples 181 and 182 clearly shows an astonishing improvement of the apoptosis efficiency of the inventive pharmaceutically active substances.

Additionally, SEQID NO 28 shows an improvement over SEQID NO 27 regarding MDA-MB-231 cells.

### Example 183

As mentioned above MDA-MB-231 cells are cultivated and harvested. The apoptosis efficiency of peptide SEQID NO 33 is measured using the subG1 method mentioned above at a concentration of the peptide of about 100 μM and an incubation time of about 24 hours. The data obtained are depicted FIG. 16.

### Example 184

As mentioned above MDA-MB-231 cells are cultivated and harvested. The apoptosis efficiency of peptide SEQID NO 40 is measured using the subG1 method mentioned above at a concentration of the peptide of about 100 μM and an incubation time of about 24 hours. The data obtained are depicted FIG. 16.

The evaluation of Examples 181 and 182 clearly shows an astonishing improvement of the apoptosis efficiency of the inventive pharmaceutically active substances. Additionally, SEQID NO 33 shows about the same efficiency as SEQID NO 40 concerning the apoptosis of MDA-MB-231 cells.

### Example 185

As mentioned above BxPC-3 cells are cultivated, harvested and evaluated using the Protocol B. The apoptosis efficiency of peptide SEQID NO 33 is measured using the subG1 method mentioned above at a concentration of the peptide of about 100 μM and an incubation time of about 24 hours.

The data obtained are depicted FIG. 17.

### Example 186

As mentioned above BxPC-3 cells are cultivated, harvested and evaluated using the Protocol B. The apoptosis efficiency of peptide SEQID NO 40 is measured using the subG1 method mentioned above at a concentration of the peptide of about 100 μM and an incubation time of about 24 hours. The data obtained are depicted FIG. 17.

The evaluation of Examples 185 and 186 clearly shows an astonishing improvement of the apoptosis efficiency of the inventive pharmaceutically active substances. Additionally, SEQID NO 33 shows an improvement over SEQID NO 40 concerning the apoptosis of BxPC-3 cells.

### Example 187

As mentioned above MDA-MB-231 cells are cultivated and harvested. The apoptosis efficiency of peptide SEQID NO 1 is measured using the subG1 method mentioned above at a concentration of the peptide of about 100 μM and an incubation time of about 24 hours. The data obtained are depicted FIG. 18.

### Example 188

As mentioned above MDA-MB-231 cells are cultivated and harvested. The apoptosis efficiency of peptide SEQID NO 28 is measured using the subG1 method mentioned above at a concentration of the peptide of about 100 μM and an incubation time of about 24 hours. The data obtained are depicted FIG. 18.

### Example 189

As mentioned above MDA-MB-231 cells are cultivated and harvested. The apoptosis efficiency of peptide SEQID NO 41 is measured using the subG1 method mentioned above at a concentration of the peptide of about 50 μM and an incubation time of about 24 hours. The data obtained are depicted FIG. 18.

The evaluation of Examples 187 to 189 clearly shows an astonishing improvement of the apoptosis efficiency of the inventive pharmaceutically active substances. Especially, Example 189 demonstrates an at least double efficiency of Peptide SEQID No 41 in view of Peptide SEQ No 1 and Peptide SEQ No 28 although the same molar amount of binding sites are present. This clearly shows an unexpected improvement of peptides comprising two, three, four, five or more of the first peptide portion, of the second peptide portion and/or of the third peptide portion, especially comprising two, three, four, five or more of the first peptide portion.

### Example 190

As mentioned above BxPC-3 cells are cultivated, harvested and evaluated using the Protocol B. The apoptosis efficiency of peptide SEQID NO 27 is measured using the subG1 method mentioned above at a concentration of the peptide of about 100 μM and an incubation time of about 24 hours. The data obtained are depicted FIG. 19.

### Example 191

As mentioned above BxPC-3 cells are cultivated, harvested and evaluated using the Protocol B. The apoptosis efficiency of peptide SEQID NO 28 is measured using the subG1 method mentioned above at a concentration of the peptide of about 100 μM and an incubation time of about 24 hours. The data obtained are depicted FIG. 19.

### Example 192

As mentioned above BxPC-3 cells are cultivated, harvested and evaluated using the Protocol B. The apoptosis efficiency of peptide SEQID NO 41 is measured using the subG1 method mentioned above at a concentration of the peptide of about 50 μM and an incubation time of about 24 hours. The data obtained are depicted FIG. 19.

The evaluation of Examples 190 to 192 clearly shows an astonishing improvement of the apoptosis efficiency of the inventive pharmaceutically active substances. Especially, Example 192 demonstrates an at least double efficiency of Peptide SEQID No 41 in view of Peptide SEQ No 27 and Peptide SEQ No 28 although the same molar amount of binding sites are present. This clearly shows an unexpected improvement of peptides comprising two, three, four, five or more of the first peptide portion, of the second peptide portion and/or of the third peptide portion, especially comprising two, three, four, five or more of the first peptide portion.

1. An pharmaceutically active substance comprising at least one peptide portion having the amino acid sequences m-v-v-y-f-r (first peptide portion), characterised in that wherein the pharmaceutically active substance comprises at least one additional amino acid and/or peptide portion at the N-terminal end and/or at the C-terminal end of the first peptide portion (second peptide portion and/or third peptide portion) being directly bound to the peptide portion having the amino acid sequences m-v-v-y-f-r (first peptide portion).

2. The pharmaceutically active substance according to claim 1, wherein the second peptide portion and/or the third peptide portion comprises at least one alpha-amino acid within a sequence of 5 amino acid units from the first peptide portion, preferably within a sequence of 3 amino acid units from the first peptide portion, the additional amino acid, and/or the peptide portion.

3. The pharmaceutically active substance according to claim 1, wherein the second peptide portion and/or the third peptide portion comprises at least one polar amino acid within a sequence of 5 amino acid units from the first peptide portion, preferably within a sequence of 3 amino acid units from the first peptide portion.

4. The pharmaceutically active substance according to claim 3, wherein the second peptide portion and/or the third peptide portion comprises at least two, preferably at least 3, polar amino acids within a sequence of 5 amino acid units from the first peptide portion, preferably within a sequence of 3 amino acid units from the first peptide portion.

5. The pharmaceutically active substance according to claim 1, wherein the second peptide portion and/or the third peptide portion comprises at least one basic amino acid within a sequence of 5 amino acid units from the first peptide portion, preferably within a sequence of 3 amino acid units from the first peptide portion.

6. The pharmaceutically active substance according to claim 5, wherein the second peptide portion and/or the third peptide portion comprises at least two, preferably at least 3, basic amino acids within a sequence of 5 amino acid units from the first peptide portion, preferably within a sequence of 3 amino acid units from the first peptide portion.

7. The pharmaceutically active substance according to claim 1, wherein the second peptide portion and/or the third peptide portion comprises more polar amino acids than nonpolar amino acids within a sequence of 5 amino acid units from the first peptide portion.

8. The pharmaceutically active substance according to claim 1, wherein the second peptide portion and/or the third peptide portion comprises the same number or more of basic amino acids than acidic amino acids within a sequence of 5 amino acid units from the first peptide portion.

9. The pharmaceutically active substance according to claim 1, wherein the pharmaceutically active substance comprises two additional amino acid and/or peptide portions, one at the N-terminal end and one at the C-terminal end of the peptide portion having the amino acid sequences m-v-v-y-f-r (second peptide portion and third portion) being directly bound to the peptide portion having the amino acid sequences m-v-v-y-f-r (first peptide portion).

10. The pharmaceutically active substance according to claim 1, wherein the second peptide portion and/or the third peptide portion comprises at least one D-amino acid.

11. The pharmaceutically active substance according to claim 10, wherein at least 50%, preferably at least 70%, of the second peptide portion and/or the third peptide portion are D-amino acids.

12. The pharmaceutically active substance according to claim 1, wherein the pharmaceutically active substance comprises at least one peptide portion having the amino acid sequences
w-m-v-v-y-f-r,
k-m-v-v-y-f-r,
m-v-v-y-f-r-w,
m-v-v-y-f-r-k,
W-m-v-v-y-f-r,
K-m-v-v-y-f-r,
m-v-v-y-f-r-W,
m-v-v-y-f-r-K.

13. The pharmaceutically active substance according to claim 12, wherein the pharmaceutically active substance comprises at least one peptide portion having the amino acid sequences
k-w-m-v-v-y-f-r,
w-k-m-v-v-y-f-r,
k-k-m-v-v-y-f-r,
w-w-m-v-v-y-f-r,
m-v-v-y-f-r-k-w,
m-v-v-y-f-r-w-k,
m-v-v-y-f-r-k-k,
m-v-v-y-f-r-w-w,
K-W-m-v-v-y-f-r,
W-K-m-v-v-y-f-r,
K-K-m-v-v-y-f-r,
W-W-m-v-v-y-f-r,
m-v-v-y-f-r-K-W,
m-v-v-y-f-r-W-K,
m-v-v-y-f-r-K-K,
m-v-v-y-f-r-W-W.

14. The pharmaceutically active substance according claim 1, wherein the pharmaceutically active substance comprises at least one peptide portion having the amino acid sequences
k-k-w-m-v-v-y-f-r,
k-w-m-v-v-y-f-r-k
or a sequence having a homology to said sequences
k-k-w-m-v-v-y-f-r
k-w-m-v-v-y-f-r-k
of at least 30%, preferably at least 60%, based on the underlined amino acids.

15. The pharmaceutically active substance according to claim 1, wherein the pharmaceutically active substance comprises at least one peptide portion having the amino acid sequences
k-k-w-m-v-v-y-f-r-k,
q-k-w-m-v-v-y-f-r-k
or a sequence having a homology to said sequences
k-k-w-m-v-v-y-f-r-k
q-k-w-m-v-v-y-f-r-k
of at least 30%, preferably at least 50%, more preferably at least 70%, based on the underlined amino acids.

16. The pharmaceutically active substance according to claim 14, wherein the pharmaceutically active substance comprises at least one peptide portion having at least one of the amino acid sequences
k-s-q-t-v-k-k-w-m-v-v-y-f-r-k,
k-k-w-m-v-v-y-f-r-k-s-s-r,
e-r-s-k-k-w-m-v-v-y-f-r-k,
k-k-w-m-v-v-y-f-r-k-e-a-r,
r-s-t-k-k-w-m-v-v-y-f-r-k,
r-s-t-k-k-w-m-v-v-y-f-r,
r-a-s-k-s-q-t-v-k-k-w-m-v-v-y-f-r-k-s-a-r,
or a sequence having a homology to said sequences
k-s-q-t-v-k-k-w-m-v-v-y-f-r-k,
k-k-w-m-v-v-y-f-r-k-s-s-r,
e-r-s-k-k-w-m-v-v-y-f-r-k,
k-k-w-m-v-v-y-f-r-k-e-a-r,
r-s-t-k-k-w-m-v-v-y-f-r-k,
r-s-t-k-k-w-m-v-v-y-f-r,
r-a-s-k-s-q-t-v-k-k-w-m-v-v-y-f-r-k-s-a-r,
of at least 50%, preferably at least 70%, based on the underlined amino acids.

17. The pharmaceutically active substance according to claim 14, wherein the pharmaceutically active substance comprises at least one peptide portion having at least one of the amino acid sequences
k-s-q-t-v-q-k-w-m-v-v-y-f-r-k,
s-q-t-v-q-k-w-m-v-v-y-f-r-k,
s-q-t-v-q-k-w-m-v-v-y-f-r,
or a sequence having a homology to said sequences
k-s-q-t-v-q-k-w-m-v-v-y-f-r-k,
s-q-t-v-q-k-w-m-v-v-y-f-r-k,
s-q-t-v-q-k-w-m-v-v-y-f-r,
of at least 50%, preferably at least 70%, based on the underlined amino acids.

18. The pharmaceutically active substance according to claim 1, wherein the pharmaceutically active substance comprises at least one peptide portion having the amino acid sequence
d-k-w-m-v-v-y-f-r-d,
or a sequence having a homology to said sequence d-k-w-m-v-v-y-f-r-d of at least 30%, preferably at least 50%, more preferably at least 70%, based on the underlined amino acids.

19. The pharmaceutically active substance according to claim 1, wherein the pharmaceutically active substance comprises a peptide chain being formed by the first peptide portion and the second and/or the third peptide portion and the peptide chain has a chain length of <100 amino acids.

20. The pharmaceutically active substance according to claim 19, wherein the pharmaceutically active substance comprises a peptide chain being formed by the first peptide portion and the second and/or the third peptide portion and the peptide chain has a chain length of <50 amino acids.

21. The pharmaceutically active substance according to claim 19, wherein the pharmaceutically active substance is a peptide that has a chain length of <100, preferably <50 amino acids.

22. The pharmaceutically active substance according to claim 1, wherein the pharmaceutically active substance comprises two, three, four, five, or more of the first peptide portion, of the second peptide portion, and/or of the third peptide portion, preferably of peptide portions having one of the amino acid sequences
w-m-v-v-y-f-r,
k-m-v-v-y-f-r,
m-v-v-y-f-r-w,
m-v-v-y-f-r-k,
W-m-v-v-y-f-r,
K-m-v-v-y-f-r,
m-v-v-y-f-r-W,
m-v-v-y-f-r-K.

23. A pharmaceutical composition, comprising at least one substance according to claim 1 or a pharmaceutically acceptable salt of this substance and a pharmaceutically acceptable carrier.

24. The pharmaceutical composition according to claim 23, wherein the pharmaceutical composition comprises at least one further medicament active ingredient, preferably a cytostatic.

25. Use of a substance according to claim 1 or of a pharmaceutically acceptable salt for preparation of a drug.

26. The use of a substance according to claim 25 or of a pharmaceutically acceptable salt for preparation of a drug for the treatment of cancer disease.

27. The use of a substance according to claim 26, wherein the cancer disease is related to cancer of head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, brain, central nervous system, solid tumors, and blood-borne tumors.

28. A drug for the treatment of cancer, comprising at least one substance according to claim 1 or a pharmaceutically acceptable salt of the pharmaceutically active substance and a pharmaceutically acceptable carrier.

29. Set (kit) consisting of separate packs of (a) an effective amount of a pharmaceutically active substance according to claim 1 and/or pharmaceutically acceptable salts, tautomers, and stereoisomers thereof, including mixtures thereof in all ratios, and (b) an effective amount of a further medicament active ingredient.

30. The substance according to claim 1, which substance is labelled.
